(19) United States
(12) Patent Application Publication
(10) Pub. No.: US 2024/0130896 A1
(43) Pub. Date: Apr. 25, 2024

(54) ADVANCED COCHLEA ACCESS

(71) Applicant: COCHLEAR LIMITED, Macquarie University, NSW (AU)

(72) Inventors: Wolfram Frederik DUECK, Macquarie University (AU); Daniel SMYTH, Macquarie University (AU)

(21) Appl. No.: 18/278,254

(22) PCT Filed: Feb. 22, 2022
(86) PCT No.: PCT/IB2022/051551

§ 371 Date: Aug. 21, 2023

Related U.S. Application Data
(60) Provisional application No. 63/152,777, filed on Feb. 23, 2021.

(51) Int. Cl. A61F 11/20; A61B 1/018; A61B 1/227; A61B 1/313

(52) U.S. Cl. CPC A61F 11/20; A61B 1/018; A61B 1/227; A61B 1/313; A61M 31/007

(57) ABSTRACT

A device, including a therapeutics substance delivery apparatus and an incisor, wherein the device is a minimally invasive inner ear therapeutic substance delivery device configured to reach the inner ear through a passage through the tympanic membrane to incise through tissue and deliver a therapeutic substance, and the incisor is at least one of a drill bit configured to drill through a promontory of a cochlea of a human, or a conduit configured to pierce a round window of the human with a fully intact round window niche.

### CROSS-REFERENCE TO RELATED APPLICATIONS

This application claims priority to U.S. Provisional Application No. 63/152,777, entitled ADVANCED COCHLEA ACCESS, filed on Feb. 23, 2021, naming Wolfram Frederik DUECK of Hannover, Germany as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a device, comprising an ear system endoscope configured to incise through tissue to reach duct(s) of an inner ear of a human and to deliver a therapeutic substance to the duct(s) through a resulting incision.

In an exemplary embodiment, there is a system comprising a processor, a sensor, and an output device, wherein the system is configured to automatically process data from the sensor indicative of sensed tissue of an ear system of a human sensed by the sensor and, using the processor, automatically identify a location in the ear system for an incision to access an area of the ear system behind the location, and provide output indicative of the identified location.

In an exemplary embodiment, there is a method, comprising accessing a middle ear of a human through a tympanic membrane with an endoscope, using an image obtained with the endoscope, guiding an incising device to a location of tissue immediately opposite an interior duct of a cochlea incising through the tissue immediate opposite the interior duct and providing a therapeutic substance into the duct through the incision.

In an exemplary embodiment, there is a device, comprising a therapeutics substance delivery apparatus and an incisor, wherein the device is a minimally invasive inner ear therapeutic substance delivery device configured to reach the inner ear through a passage through the tympanic membrane no greater than 10 mm in diameter to incise through tissue and deliver a therapeutic substance, and the incisor is at least one of a drill bit configured to drill through a promontory of a cochlea of a human, or a conduit configured to pierce a round window of the human with a fully intact round window niche.

In an exemplary embodiment, there is an ear system endoscope, including a handle, a termination including a lumen, a means for incising through tissue, a fiber optics channel, a surgical tool passageway configured to receive a long narrow surgical tool, which surgical tool passageway is in channel communication with the lumen, a light source, and an irrigation conduit, wherein the ear system endoscope is configured to reach duct(s) of an inner ear of a human through an incision established by the means for incising through tissue and to deliver a therapeutic substance to the duct(s) through a resulting incision.

### BRIEF DESCRIPTION OF THE DRAWINGS

Embodiments are described below with reference to the attached drawings, in which:

Embodiments are described below with reference to the attached drawings, in which:

FIG. 1 is a perspective view of an exemplary hearing prosthesis;

FIGS. 2A-2H are views of exemplary electrode arrays to which the teachings detailed herein can be applicable;

FIGS. 3A and 3B are side and perspective views of an electrode assembly extended out of an embodiment of an insertion sheath of the insertion tool illustrated in FIG. 2;

FIGS. 4A-4E are simplified side views depicting an exemplary insertion process of the electrode assembly into the cochlea;

FIGS. 5-7 present an exemplary embodiment of an exemplary therapeutic substance delivery system;

FIG. 8 presents an ear system endoscope according to an exemplary embodiment;

FIG. 9 presents the ear system endoscope of FIG. 8 in use;

FIGS. 10 to 16 present various features of various embodiments and/or use thereof;

FIG. 17 presents an exemplary drill bit according to an embodiment;

FIGS. 18-25 present various features of various embodiments and/or use thereof;

FIG. 26 presents an exemplary view;

FIG. 27 presents an exemplary system;

FIG. 28 presents an exemplary flowchart for an exemplary method;

FIGS. 29A and 29B present exemplary tools;

FIGS. 30A and 30B present an exemplary assembly;

FIGS. 31-36 present some exemplary embodiments of hardware for implementing some of the teachings detailed herein; and

FIGS. 37-41 present additional exemplary teachings.

### DETAILED DESCRIPTION

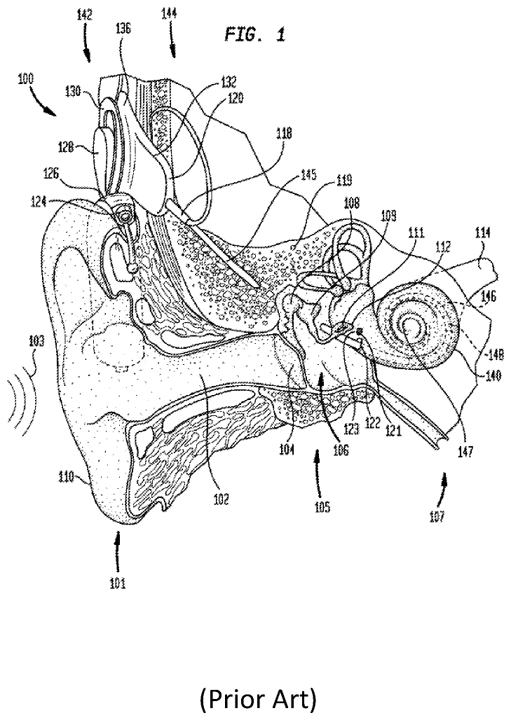

FIG. 1 is a perspective view of an exemplary cochlear implant 100 implanted in a recipient having an outer ear 101, a middle ear 105, and an inner ear 107. In a fully functional ear, outer ear 101 comprises an auricle 110 and an ear canal 102. Acoustic pressure or sound waves 103 are collected by auricle 110 and channeled into and through ear canal 102. Disposed across the distal end of ear canal 102 is a tympanic membrane 104 that vibrates in response to sound waves 103. This vibration is coupled to oval window or fenestra ovalis 112 through the three bones of the middle ear 105, collectively referred to as the ossicles 106, and comprising the malleus 108, the incus 109, and the stapes 111. Ossicles 106 filter and amplify the vibrations delivered by tympanic membrane 104, causing oval window 112 to articulate, or vibrate. This vibration sets up waves of fluid motion of the perilymph within cochlea 140. Such fluid motion, in turn, activates hair cells (not shown) inside the cochlea which in turn causes nerve impulses to be generated which are transferred through spiral ganglion cells (not shown) and auditory nerve 114 to the brain (also not shown) where they are perceived as sound.

The exemplary cochlear implant illustrated in FIG. 1 is a partially-implanted stimulating medical device. Specifically, cochlear implant 100 comprises external components 142 attached to the body of the recipient, and internal or implantable components 144 implanted in the recipient. External components 142 typically comprise one or more sound input elements for detecting sound, such as microphone 124, a sound processor (not shown), and a power source (not shown). Collectively, these components are housed in a behind-the-ear (BTE) device 126 in the example depicted in FIG. 1. External components 142 also include a transmitter unit 128 comprising an external coil 130 of a transcutaneous energy transfer (TET) system. Sound processor 126 processes the output of microphone 124 and generates encoded stimulation data signals which are provided to external coil 130.

Internal components 144 comprise an internal receiver unit 132 including a coil 136 of the TET system, a stimulator unit 120, and an elongate stimulating lead assembly 118. Internal receiver unit 132 and stimulator unit 120 are hermetically sealed within a biocompatible housing commonly referred to as a stimulator/receiver unit. Internal coil 136 of receiver unit 132 receives power and stimulation data from external coil 130. Stimulating lead assembly 118 has a proximal end connected to stimulator unit 120, and extends through mastoid bone 119. Lead assembly 118 has a distal region, referred to as electrode assembly 145, a portion of which is implanted in cochlea 140.

Electrode assembly 145 can be inserted into cochlea 140 via a cochleostomy 122, or through round window 121, oval window 112, promontory 123, or an opening in an apical turn 147 of cochlea 140. Integrated in electrode assembly 145 is an array 146 of longitudinally-aligned and distally extending electrode contacts 148 for stimulating the cochlea by delivering electrical, optical, or some other form of energy. Stimulator unit 120 generates stimulation signals each of which is delivered by a specific electrode contact 148 to cochlea 140, thereby stimulating auditory nerve 114.

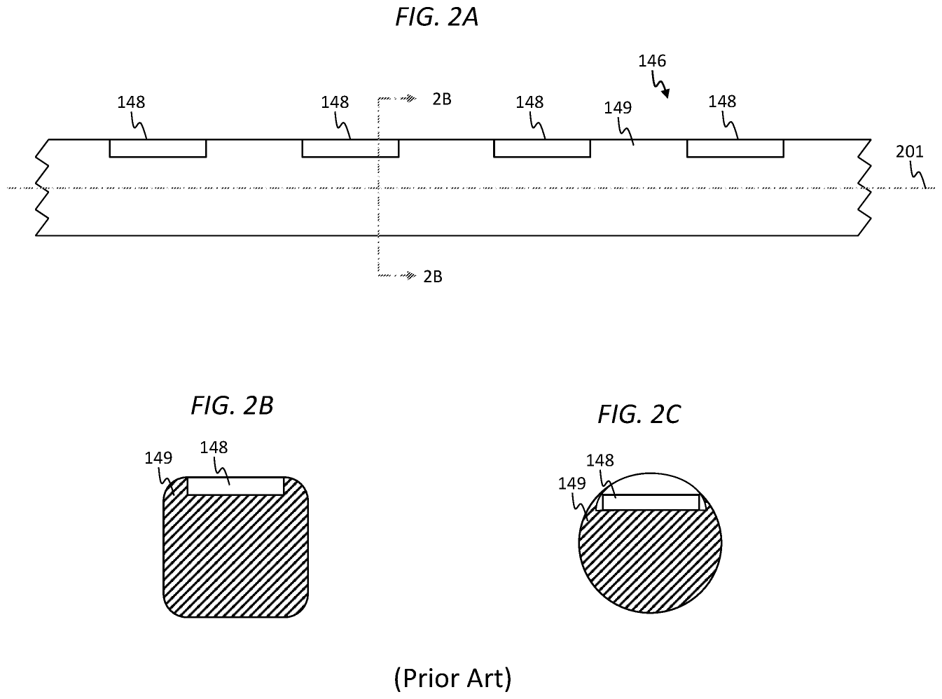

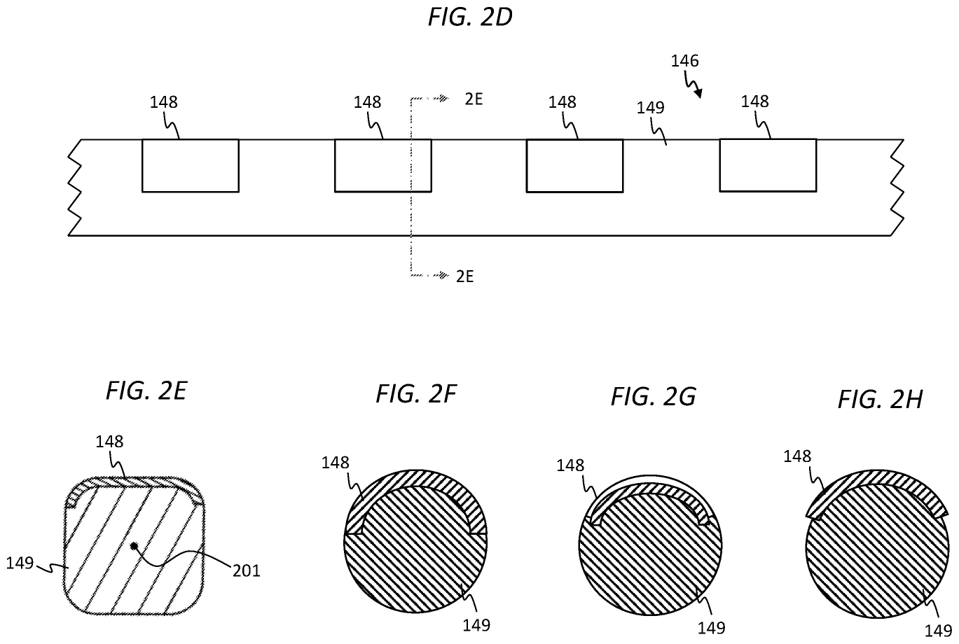

FIG. 2A depicts a conceptual side view of a portion of electrode array 146, depicting four electrode contacts 148 evenly spaced along a longitudinal axis of the electrode array 146. It is noted that in some alternate embodiments, the electrode is not evenly spaced. FIG. 2B depicts a conceptual cross-sectional view through one of the electrode contacts 148, which also depicts the carrier 149 of the electrode contact 148. In an exemplary embodiment, the carrier 149 is made of silicone. Not depicted in the figures are electrical leads and stiffener components that are sometimes embedded in the carrier 149. The embodiment of FIG. 2B represents an electrode array 146 that has a generally rectangular cross-section. FIG. 2C depicts an alternate embodiment where the electrode array 146 has a generally circular cross-section. It is also noted that in some exemplary embodiments, the cross-section is oval shaped. Thus, the embodiment of FIGS. 2A-2C is a species of the genus of an electrode array having a generally continuously curving cross-section. Any electrode array of any cross-section or any configuration can be utilized with the teachings detailed herein.

The electrode contacts 148 depicted in FIGS. 2A-2C are so-called flat contacts. In this regard, the surface of the electrode contact that faces the wall of the cochlea/the faces away from the longitudinal axis of the electrode array 146 is flat. Conversely, as seen in FIGS. 2D-2H, in some alternate embodiments, the electrode contacts 148 are so-called half band electrodes. In some exemplary embodiments, a band of contact material is “smashed” or otherwise compressed into a “half band,” as seen in the figures. It is noted that by “half band,” this does not mean that the electrode contact must necessarily span half of the outside diameter of the electrode array, as is the case in FIGS. 2G and 2H. The term is directed towards the configuration of the electrode itself as that term has meaning in the art. Any electrode contact that can have utilitarian value according to the teachings detailed herein can be utilized in at least some exemplary embodiments.

As can be seen from FIGS. 2A-2H, the positioning of the electrode contacts relative to the carrier 149 can vary with respect to alignment of the outer surface of the carrier with the outer surface of the contact. For example, FIGS. 2A, 2E, and 2F depict the outer surface of the contacts 148 as being flush with the outer surface of the carrier 149. Conversely, FIGS. 2C and 2G depict the contact 148 as being recessed with respect to the outer surface of the carrier 149, while FIG. 2H depicts the contact 148 as being proud relative to the outer surface of the contact 149. It is noted that these various features are not limited to the specific contact geometry and/or the specific carrier geometry depicted in the figures, and that one or more features of one exemplary embodiment can be combined with one or more features of another exemplary embodiment. For example, while FIG. 2H depicts a half band contact as being proud of the carrier 149 having a generally circular cross-section, a flat electrode such as that depicted in FIG. 2A can be proud of the carrier as well.

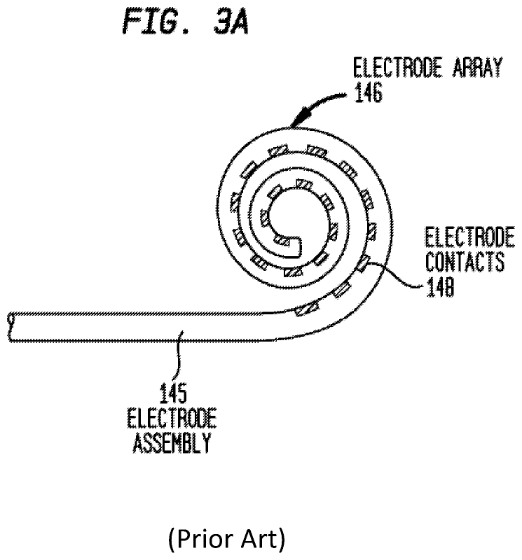

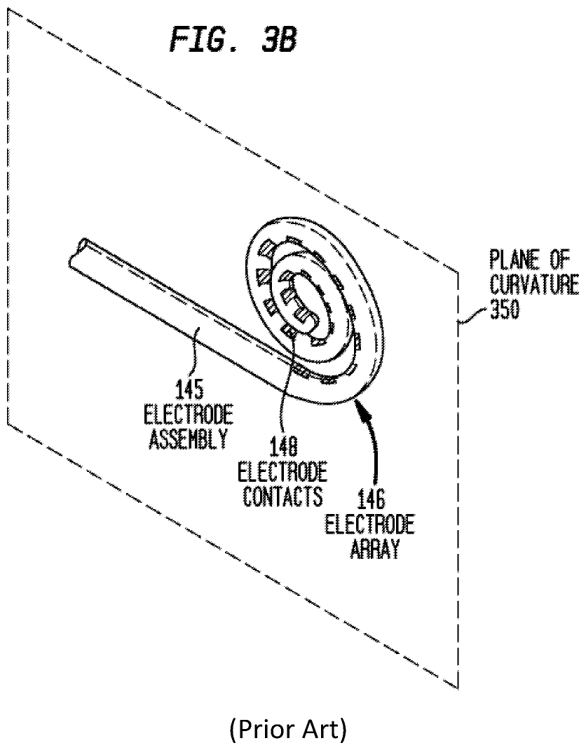

FIGS. 3A and 3B are side and perspective views, respectively, of representative electrode assembly 145. As noted, electrode assembly 145 comprises an electrode array 146 of electrode contacts 148. Electrode assembly 145 is configured to place electrode contacts 148 in close proximity to the ganglion cells in the modiolus. Such an electrode assembly, commonly referred to as a perimodiolar electrode assembly, is manufactured in a curved configuration as depicted in FIGS. 3A and 3B. When free of the restraint of a stylet or insertion guide tube, electrode assembly 145 takes on a curved configuration due to it being manufactured with a bias to curve, so that it is able to conform to the curved interior of cochlea 140. As shown in FIG. 3B, when not in cochlea 140, electrode assembly 145 generally resides in a plane 350 as it returns to its curved configuration. That said, it is noted that the teachings detailed herein and/or variations thereof can be applicable to a so-called straight electrode array, which electrode array does not curl after being free of a stylet or insertion guide tube etc., but instead remains straight. It is noted that when in the cochlea, the electrode assembly 145 takes on a conical shape with respect to plane 350 in that it can be described as winding upward away from the plane 350 about an axis normal thereto, owing to the shape of the cochlea (more on this below).

The perimodiolar electrode assembly 145 of FIGS. 3A and 3B is pre-curved in a direction that results in electrode contacts 148 being located on the interior of the curved assembly, as this causes the electrode contacts to face the modiolus when the electrode assembly is implanted in or adjacent to cochlea 140.

It is also noted that while the embodiments of FIGS. 2A-3B have been presented in terms of a so-called non-tapered electrode array (where the cross-sections of the array on a plane normal to the longitudinal axis at various locations along the longitudinal axis (e.g., in between each electrode (or a majority of the electrodes), in the middle of each electrode (or a majority of the electrodes) etc.) have generally the same cross-sectional area and shape), in an alternate embodiment, the teachings detailed herein can be applicable to a so-called tapered electrode, where the cross-sectional areas on planes taken normal to the longitudinal axis decrease with location towards the distal end of the electrode array.

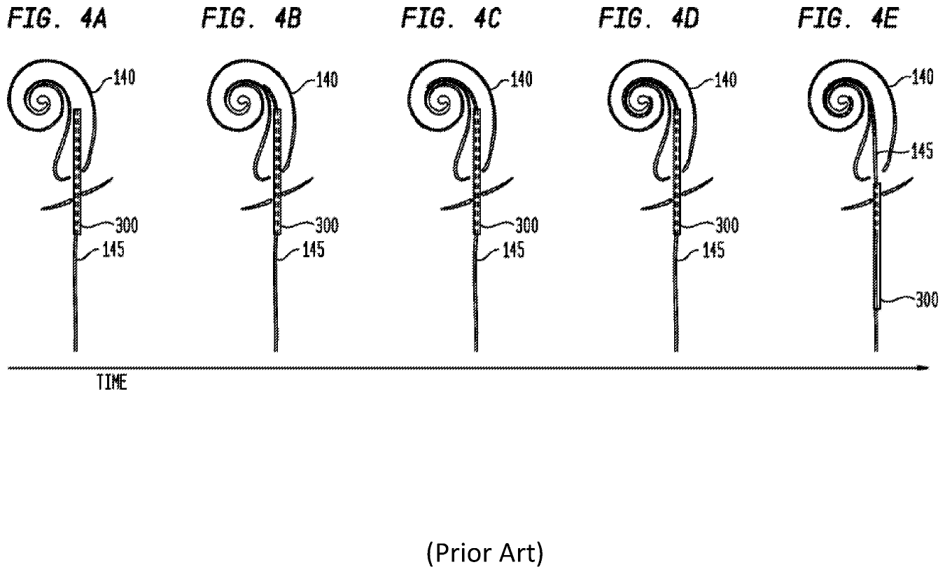

FIGS. 4A-4E depict an exemplary insertion regime of an electrode assembly according to an exemplary embodiment. As shown in FIG. 4A, the combined arrangement of an insertion guide tube 300 and electrode assembly 145 is substantially straight. This is due in part to the rigidity of insertion guide tube 300 relative to the bias force applied to the interior wall of the guide tube by pre-curved electrode assembly 145.

As noted, in some embodiments, the electrode assembly 145 is biased to curl and will do so in the absence of forces applied thereto to maintain the straightness. That is, electrode assembly 145 has a memory that causes it to adopt a curved configuration in the absence of external forces. As a result, when electrode assembly 145 is retained in a straight orientation in guide tube 300, the guide tube prevents the electrode assembly from returning to its pre-curved configuration. In the embodiment configured to be implanted in scala tympani of the cochlea, electrode assembly 145 is pre-curved to have a radius of curvature that approximates and/or is less than the curvature of medial side of the scala tympani of the cochlea. Such embodiments of the electrode assembly are referred to as a perimodiolar electrode assembly, and this position within cochlea 140 is commonly referred to as the perimodiolar position. In some embodiments, placing electrode contacts in the perimodiolar position provides utility with respect to the specificity of electrical stimulation, and can reduce the requisite current levels thereby reducing power consumption.

As shown in FIGS. 4B-4D, electrode assembly 145 may be continually advanced through insertion guide tube 300 while the insertion sheath is maintained in a substantially stationary position. This causes the distal end of electrode assembly 145 to extend from the distal end of insertion guide tube 300. As it does so, the illustrative embodiment of electrode assembly 145 bends or curves to attain a perimodiolar position, as shown in FIGS. 4B-4D, owing to its bias (memory) to curve. Once electrode assembly 145 is located at the desired depth in the scala tympani, insertion guide tube 300 is removed from cochlea 140 while electrode assembly 145 is maintained in a stationary position. This is illustrated in FIG. 4E.

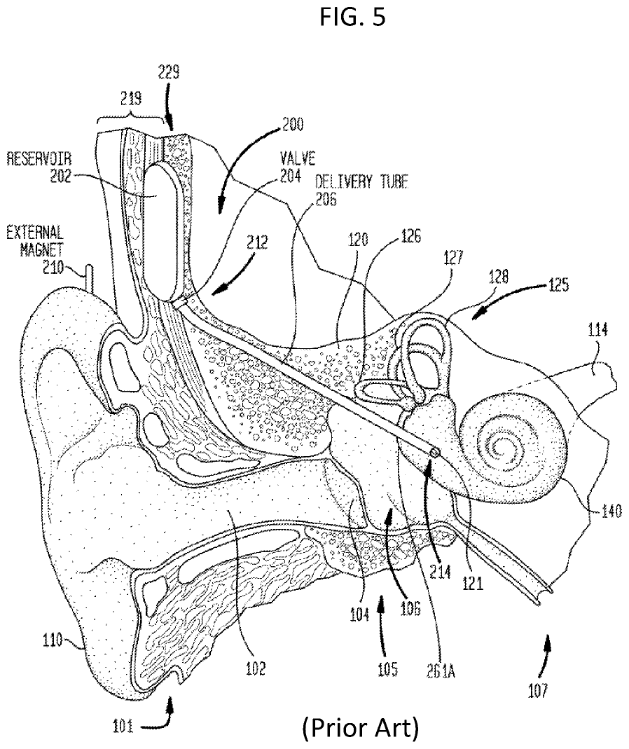

FIG. 5 depicts an exemplary drug delivery device, the details of which will be provided below. As shown in FIG. 5, semicircular canals 125 are three half-circular, interconnected tubes located adjacent cochlea 140. Vestibule 129 provides fluid communication between semicircular canals 125 and cochlea 140. The three canals are the horizontal semicircular canal 126, the posterior semicircular canal 127, and the superior semicircular canal 128. The canals 126, 127, and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

It can be utilitarian to have a prompt and/or extended delivery solution for use in the delivery of treatment substances to a target location of a recipient. In general, extended treatment substance delivery refers to the delivery of treatment substances over a period of time (e.g., continuously, periodically, etc.). The extended delivery may be activated during or after surgery and can be extended as long as is needed. The period of time may not immediately follow the initial implantation of the auditory prosthesis. Embodiments of the teachings herein can facilitate extended delivery of treatment substances, as well as facilitating prompt delivery of such substances.

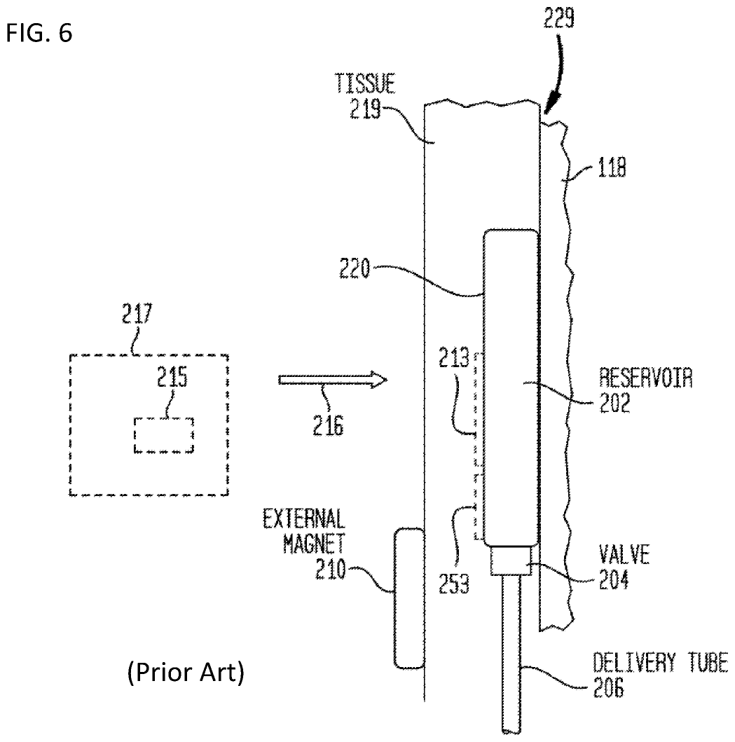

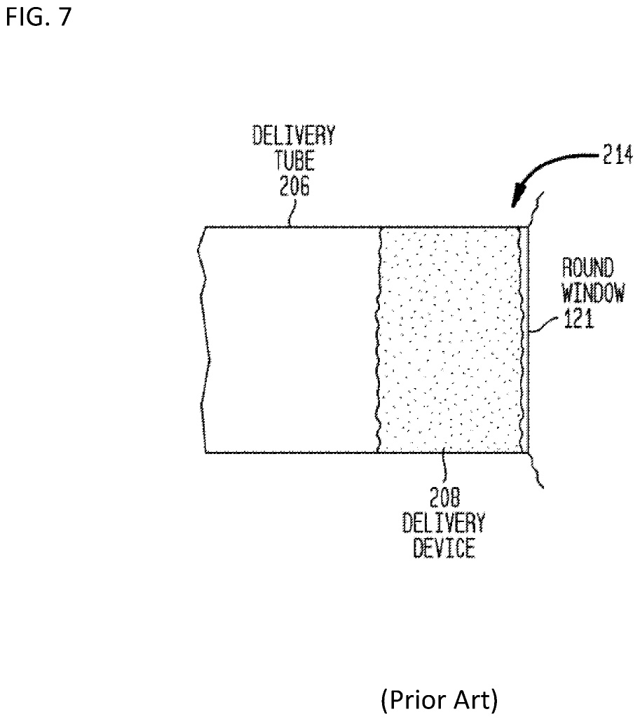

FIG. 5 illustrates an implantable delivery system 200 that can be utilized with the teachings detailed herein, and otherwise modified as detailed by way of example below. The delivery system has a passive actuation mechanism. However, it is noted that the delivery system 200 can also or instead have an active actuation system. The delivery system 200 is sometimes referred to herein as an inner ear delivery system because it is configured to deliver treatment substances to the recipient's inner ear (e.g., the target location is the interior of the recipient's cochlea 140 (in other embodiments, this could be provided to the semi-circular canals). FIG. 6 illustrates a first portion of the delivery system 200, while FIG. 7 is a cross-sectional view of a second portion of the delivery system 200.

Delivery system 200 of FIGS. 5-7 comprises a reservoir 202, a valve 204, a delivery tube 206, and a delivery device 208 (FIG. 7). The delivery system 200 can include, or operate with, an external magnet 210. For ease of illustration, the delivery system 200 is shown separate from any implantable auditory prostheses. However, it is to be appreciated that the delivery system 200, and any of the other delivery systems detailed herein and/or variations thereof, could be used with, for example, cochlear implants, such as that presented in FIG. 1, direct acoustic stimulators, middle ear implants, bone conduction devices, etc. The implantable components (e.g., reservoir, valve, delivery tube, etc.) of delivery system 200 (or any other delivery system detailed herein) could be separate from or integrated with the other components of the implantable auditory prosthesis.

The reservoir 202 is positioned within the recipient underneath a portion of the recipient's skin/muscle/fat, collectively referred to herein as tissue 219. The reservoir 202 may be positioned between layers of the recipient's tissue 219 or may be adjacent to a subcutaneous outer surface 229 of the recipient's skull. For example, the reservoir 202 may be positioned in a surgically created pocket at the outer surface 229 (i.e., adjacent to a superior portion 118 of the temporal bone 115).

The reservoir 202 is, prior to or after implantation, at least partially filled with a treatment substance for delivery to the inner ear 107 of the recipient. The treatment substance may be, for example, in a liquid form, a gel form, and/or comprise nanoparticles or pellets. In certain arrangements, the treatment substance may initially be in a crystalline/solid form that is subsequently dissolved. For example, a reservoir could include two chambers, one that comprises a fluid (e.g., artificial perilymph or saline) and one that comprises the crystalline/solid treatment substance. The fluid may be mixed with the crystalline/solid treatment substance to form a fluid or gel treatment substance that may be subsequently delivered to the recipient.

The reservoir 202 includes a needle port (not shown) so that the reservoir 202 can be refilled via a needle injection through the skin. The reservoir 202 may be explanted and replaced with another reservoir that is, prior to or after implantation, at least partially filled with a treatment substance. The reservoir 202 may have a preformed shape and the reservoir is implanted in this shape. The reservoir 202 may have a first shape that facilitates implantation and a second shape for use in delivering treatment substances to the recipient. For example, the reservoir 202 may have a rolled or substantially flat initial shape that facilitates implantation. The reservoir 202 may then be configured to expand after implantation. Such may be used, for example, to insert the reservoir through a tympanostomy into the middle ear or ear canal, through an opening in the inner ear, or to facilitate other minimally invasive insertions.

The delivery tube 206 includes a proximal end 212 and a distal end 214. The proximal end 212 of the delivery tube 206 is fluidically coupled to the reservoir 202 via the valve 204. As shown in FIG. 7, the distal end 214 of the delivery tube 206 is fluidically coupled to the recipient's round window 121. A delivery device 208 disposed within the distal end 214 of the delivery tube 206 is positioned abutting the round window 121. As described further below, the delivery tube 206 may be secured within the recipient so that the distal end 214 remains located adjacent to the round window 121.

FIGS. 5-7 illustrate a system that utilizes utilize a passive actuation mechanism to produce a pumping action to transfer a treatment substance from the reservoir 202 to the delivery device 208 at the distal end 214 of the delivery tube 206. More specifically, in this system, the reservoir 202 is compressible in response to an external force 216. That is, at least one part or portion of the reservoir 202, such as wall 220 or a portion thereof, is formed from a resiliently flexible material that is configured to deform in response to application of the external force 216. In some implementations of the system of FIG. 5, positioning of the reservoir 202 adjacent the superior portion of the mastoid provides a rigid surface that counters the external force 216. As a result, a pressure change occurs in the reservoir 202 so as to propel (push) a portion of the treatment substance out of the reservoir through valve 204.

FIGS. 5 and 6 illustrate a specific arrangement in which the reservoir 202 includes a resiliently flexible wall 220. It is to be appreciated that the reservoir 202 can be formed from various resiliently flexible parts and rigid parts. It is also to be appreciated that the reservoir 202 may have a variety of shapes and sizes (e.g., cylindrical, square, rectangular, etc.) or other configurations. For example, the reservoir 202 could further include a spring mounted base that maintains a pressure in the reservoir 202 until the reservoir is substantially empty. Other mechanisms for maintaining a pressure in the reservoir may be used in other arrangements.

The external force 216 is applied manually using, for example, a user's finger. The user (e.g., recipient, clinician, caregiver, etc.) may press on the tissue 219 adjacent to the reservoir 202 to create the external force 216. A single finger press may be sufficient to propel the treatment substance through valve 204. In some instances, a multiple finger presses may be used to create a pumping action that propels the treatment substance from the reservoir 202.

The external force 216 is applied through a semi-manual method that uses an external actuator 217 (FIG. 2B). That is, the external actuator 217 may be pressed onto the soft tissue 219 under which the reservoir 202 is located. The movement (e.g., oscillation/vibration) of the actuator 217 deforms the reservoir 202 to create the pumping action that propels the treatment substance out of the reservoir.

Internal and/or external magnets and/or magnetic materials may be used in the arrangements of FIGS. 5 and 6 to ensure that the actuator 217 applies force at an optimal location of the reservoir 202. For example, the reservoir 202 may include a magnetic positioning member 213 located at or near an optimal location for application of an external force from the actuator 217. The actuator 217 may include a magnet 215 configured to magnetically mate with the magnetic positioning member 213. As such, when actuator 217 is properly positioned, the magnet 215 will mate with the magnetic positioning member 213 and the force from the actuator 217 will be applied at the optimal location.

A remote control, remotely placed actuator (subcutaneous or otherwise) may be alternatively used. For example, in a further arrangement, the implant includes implanted electronics 253 (shown using dotted lines in FIG. 6). These implanted electronics 253 may be configured to, for example, control the valve 204 and/or include an actuation mechanism that can force treatment substance from the reservoir 202. The implanted electronics 253 may be powered and/or controlled through a transcutaneous link (e.g., RF link). As such, the implanted electronics 253 may include or be electrically connected to an RF coil, receiver/transceiver unit, etc.

The implanted electronics 253 may include or be connected to a sensor that is used, at least in part, to assist in control of delivery of the treatment substance to the recipient. For example, a sensor (e.g., a temperature sensor, a sensor to detect infection or bacteria growth, etc.) may provide indications of when a treatment substance should be delivered and/or when delivery should be ceased for a period of time. A sensor may also be configured to determine an impact of the treatment substance on the recipient (e.g., evaluate effectiveness of the treatment substance).

As noted, the treatment substance (sometimes herein referred to as therapeutic substance) is released from the reservoir 202 through the valve 204. The valve 204 may be a check valve (one-way valve) that allows the treatment substance to pass therethrough in one direction only. This assures that released treatment substances do not back-flow into the reservoir 202. The valve 204 is a valve that is configured to open in response to the pressure change in the reservoir 202 (e.g., a ball check valve, diaphragm check valve, swing check valve or tilting disc check valve, etc.). The valve 204 may be a stop-check valve that includes an override control to stop flow regardless of flow direction or pressure. That is, in addition to closing in response to backflow or insufficient forward pressure (as in a normal check valve), a stop-check value can also be deliberately opened or shut by an external mechanism, thereby preventing any flow regardless of forward pressure. The valve 204 may be a stop-check value that is controlled by an external electric or magnetic field generated by, for example, the external magnet 210, an electromagnet, etc. In the system of FIGS. 5 and 6, the valve is responsive to a magnetic field generated by external magnet 210. As such, the valve 204 will temporarily open when the external magnet 210 is positioned in proximity to the valve 204 and will close when the external magnet 210 is removed from the proximity of the valve 204. Variable magnet strengths of external magnets may be used to control the dosage of the treatment substance. Additionally, an electromagnet may be used in place of the external magnet 210.

The use of a stop-check valve can prevent unintended dosing of the treatment substance when, for example, an accidental external force acts on the reservoir 202. The reservoir 202 is formed such that an increase in pressure of the reservoir 202 without an accompanying treatment substance release will not damage (i.e., rupture) the reservoir.

The use of a magnetically activated stop-check valve is merely exemplary and that other types of valves may be used. For example, the valve 204 may be actuated (i.e., opened) in response to an electrical signal (e.g., piezoelectric valve). The electrical signal may be received from a portion of an auditory prosthesis (not shown) that is implanted with the delivery system 200 or the electrical signal may be received from an external device (e.g., an RF actuation signal received from an external sound processor, remote control, etc.). In some instances, manually applied (e.g., finger) force be also able to open the valve 204.

Once the treatment substance is released through valve 204, the treatment substance flows through the delivery tube 206 to the delivery device 208. The delivery device 208 operates as a transfer mechanism to transfer the treatment substance from the delivery tube 206 to the round window 121. The treatment substance may then enter the cochlea 140 through the round window 121 (e.g., via osmosis). The delivery device 208 may be, for example, a wick, a sponge, permeating gel (e.g., hydrogel), etc.

The reservoir 202 may include a notification mechanism that transmits a signal or notification indicating that the reservoir 202 is substantially empty and/or needs refilled. For example, one or more electrode contacts (not shown) may be present and become electrically connected when the reservoir is substantially empty. Electronic components associated with or connected to the reservoir 202 may accordingly transmit a signal indicating that reservoir needs filled or replaced.

FIGS. 5-7 illustrate a specific example in which the round window 121 is the target location. As noted above, the round window 121 is an exemplary target location and other target locations are possible. FIGS. 5-7 also illustrate that the reservoir 202 is positioned adjacent to the outer surface 229 of the recipient's skull so that an external force may be used to propel the treatment substance from the reservoir.

While the features of FIGS. 5-7 are directed to an implantable therapeutic substance delivery system of the prior art, embodiments disclosed herein are directed towards medical tools and methods utilized by surgeons or other healthcare professionals, and, in some embodiments, by recipients or lay people (more on this below) to provide therapeutic substances to the cochlea, and, to tools and methods that provide access to the cochlea (in the first instance or on a repeated or subsequent to the first instance basis). That is, in contrast to the embodiment of FIG. 5, these teachings are directed towards a non-implantable device, although it is noted that in some embodiments, there may be an implanted component involved with the methods. In a broad sense, the teachings below are directed to tools, not implants, in the sense that one does not implant a tool, but one may utilize a tool in conjunction with an implant. It is noted that some embodiments can use one or more of the aspects of the embodiment of FIG. 5 to enable the teachings below.

It is noted that in some exemplary embodiments, the therapeutic substance can be delivered to other portions of the ear system, such as by way of example only and not by way of limitation, the semicircular canals. Embodiments include the utilization of the techniques and/or tools etc., herein to pierce or otherwise in size the tissue of the semicircular canals, and provide therapeutic substance there to. It is noted that any disclosure herein referencing accessing and/or providing therapeutic substance to the ducts of the cochlea corresponds to an alternate disclosure of an alternate exemplary embodiment of accessing and/or providing their picks options to the semicircular canals providing that the art enables such, unless otherwise noted.

Note also that while the various teachings detailed herein are directed towards piercing or otherwise incising through the tympanic membrane and or the round window membrane, in an exemplary embodiment, the teachings detailed herein also provide an arrangement that can enable incising through so-called false membranes as well as the true membrane.

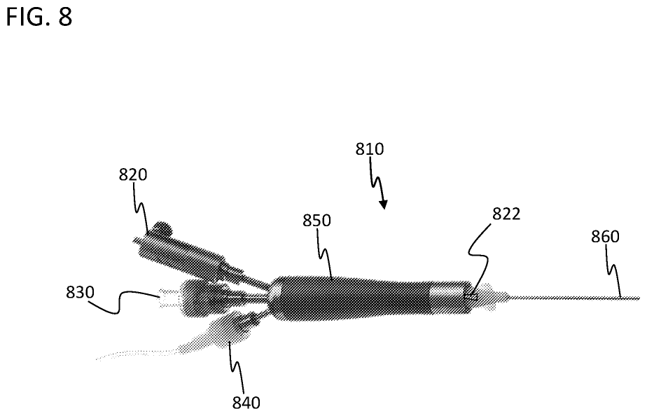

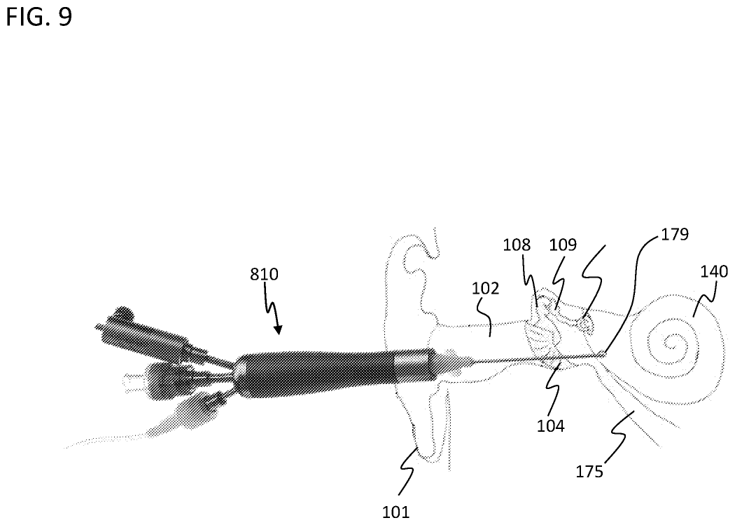

In this regard, FIG. 8 presents an exemplary embodiment of an ear system endoscope 810. The ear system endoscope is configured to incise through tissue to reach duct(s) of an inner ear of a human and to deliver therapeutic substance to the duct(s) through a resulting incision. More specifically, as seen in FIG. 9, the ear system endoscope 810 can be seen extending into the outer ear 102/ear canal 102, with a component penetrating the tympanic membrane 104, which component extends all the way to the round window niche 179. It is briefly noted that while the embodiments disclosed above have generally identified that area labeled 179 as the round window, in reality, the round window is eclipsed at least partially by the promontory 123 or otherwise by the bone that establishes the round window niche 179. In this exemplary embodiment, the component is curved and/or angled at the end so as to bend around the round window niche and pierce the round window (the component can be flexible—more on this below). More features of this will be described below, but briefly, upon piercing the round window, a therapeutic substance can be delivered into the duct of the cochlea, and thus embodiments include methods of utilizing the ear system endoscope 810 to provide therapeutic substance into the ducts of the cochlea from a location outside the middle ear, and from outside the outer ear for that matter in some embodiments, through the middle ear, and into the inner ear. Briefly, in an exemplary embodiment, the therapeutic substance on a molecular basis moves from the location outside the middle ear and/or from outside the outer ear, to the inner ear, within 1 minute, within 45, 30, 25, 20, 15, 10 or 5 seconds or less. This as opposed to, for example, that which may occur utilizing diffusion or the like where the therapeutic substances simply provided to the round window niche, and the therapeutic substance diffuses through the round window into the cochlea.

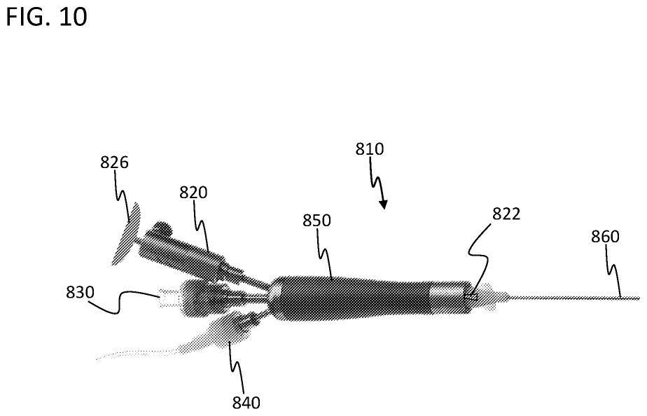

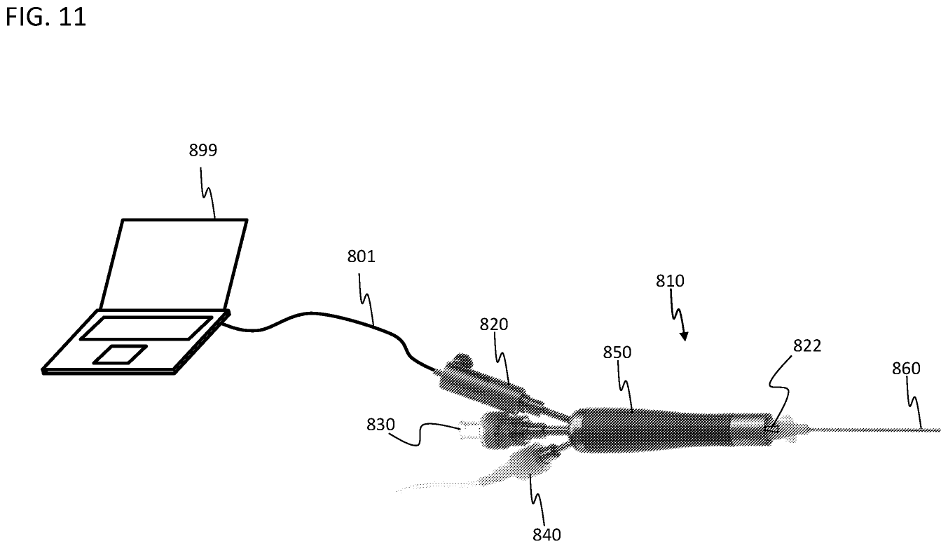

Returning back to FIG. 8, the ear system endoscope 810 includes various components, such as, an optical channel 820 which enables data indicative of an image inside the ear system to be conveyed to a location outside the ear system. This can be based on fiber optics or wired communication. Corollary to this is that there is a camera 822 or some other light capture arrangement (a purely optical device can be used, which may magnify light captured at the working end) that is part of the ear system endoscope, that is in electrical communication or light communication with the optical channel 820. The optical channel 820 can be in communication with a display 826 (see FIG. 10) or some other image conveying device, such as a lens (again, a purely optical system can be used, akin to the output of a traditional microscope, for example). Alternatively in or in addition to this, the optical channel 820 can be configured to provide the data indicative of an image via a cable 801 (or a wireless link, such as a Blue Tooth link) to a “remote” monitor or the like positioned away from the ear system endoscope 810, such as a monitor of a laptop 899 (See FIG. 11) or a desktop computer, which could be located in the same room in which the ear system endoscope is being utilized to reach the cochlea. Accordingly, it can be seen that in some scenarios of use the surgeon or other healthcare professional or whoever is utilizing the ear system endoscope guides the endoscope through the ear system by looking at the endoscope/looking in the direction of the side of the human's head/looking at the outer ear, while in some other embodiments, the guiding is executed while the user is looking at a computer screen and thus looking in a direction away from the human's head/outer ear, etc.

Again referring back to FIG. 8, the ear system endoscope 810 further includes a surgical tool port 830. This port can be configured to receive a needle and/or a drill and/or a laser generator and/or output and/or or a micro tweezers or can just be a general port to which a therapeutic substance reservoir or a therapeutic substance supply line can be attached to deliver therapeutic substance to the inner ear. Furthermore, in an example, the port can receive a biopsy tool and/or blades and/or forceps and/or microneedles (microneedle array/assembly), catheters, etc. Moreover, by way of example only and not by way of limitation, in an exemplary embodiment, tools that are configured to take a sample of a fluid or a liquid within the body, such as perilymph within the cochlea and/or the fluid within the semicircular canals, example, can be extended through the aforementioned port(s).

An exemplary embodiment includes a steerable tip endoscope with a channel for vision and a channel for tools (e.g., a drill tip and/or a therapeutic substance delivery tool (e.g., a conduit extends through a channel), the tool bending in compliance with the tip angle. Thus, there could be 2, 3, 4 or more surgical tool ports 830.

Also, as can be seen, there is an irrigation port 840, which can be utilized to provide irrigation fluid, such as a saline liquid, to the working end of the ear system endoscope, which can be used to provide irrigation of the middle and/or inner ear during use of the tool. The tympanic membrane can also be irrigated utilizing the irrigation features of the ear system endoscope in some embodiments.

As can be seen, the channel 820 and the ports 830 and 840 are supported by a body 850, which can be ergonomically designed so that a surgeon or other healthcare professional and easily grip and support the ear system endoscope with a thumb and one or more fingers of a hand, or the entire hand.

The working end of the ear system endoscope 810 includes a termination 860, which can be a tube made of metal, such as stainless steel (e.g., 316), or some other material. In an exemplary embodiment, the termination 860 can correspond to those of at least the body portion (which may or may not include the sharp end) of a syringe termination approved for use in the United States as of Feb. 21, 2021. This can be a low volume, medium volume, or high volume termination. In an exemplary embodiment, the termination is sized and dimensioned the termination can extend from the tympanic membrane 104 to the promontory (and into the promontory) and/or to the round window niche of the cochlea, when the base 850 is inserted into the ear canal and/or is located at the beginning of the ear canal (as seen in FIG. 9).

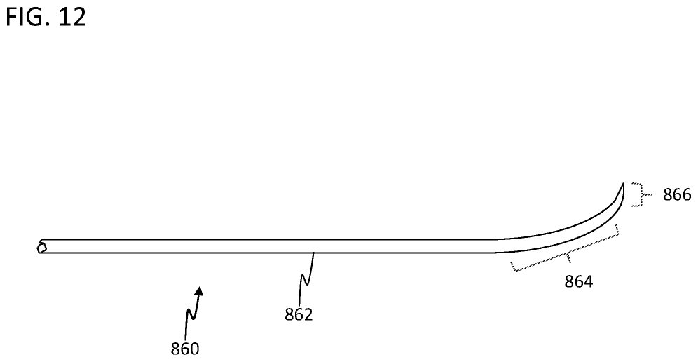

FIG. 12 depicts a portion of the termination 860. The termination 860 has a straight part and a curved part, as can be seen. In this exemplary embodiment, this can enable access to the round window through the round window niche when the termination is extended into the middle ear through the tympanic membrane. That is, the curved portion can curve around the overhang in the round window niche to reach the round window. The end of the termination 860 is configured, in at least some embodiments, in the manner that the ends of other terminations for syringes are formed (e.g., the angled cut across the tube).

In an exemplary embodiment, the termination 860 can be a rigid structure, while in other embodiments, the termination can be flexible, or at least a portion can be flexible. In an exemplary embodiment, the termination can be an assembly or a compilation that has a rigid part 862 and a flexible part 864. In this exemplary embodiment, the overall termination has a sufficiently rigid structure that will enable the termination to pierce the tympanic membrane, and then upon reaching the round window niche, the flexible portion will flex upon interaction with the tissue of the round window niche so that it can be driven to the round window and thus pushed through the round window so as to establish communication from outside the outer ear to inside the cochlea.

Note that while in some embodiments, the component that interacts with the round window can often be the conduit as disclosed herein, which conduit extends out of the termination. In other embodiments, it is the termination that interacts with the round window. Indeed, as will be detailed below, in some embodiments, there is no separate conduit. Moreover, in an exemplary embodiment, as noted herein, the termination to have varying diameters, such as a smaller outer diameter so as to accommodate the area within the round window niche, etc., while the larger diameter will be utilized over the remainder of the tool to provide structural support in a manner consistent with the traditional use of a termination of an endoscope.

In an exemplary embodiment, the flexible and/or bendable portions can be controlled via a guidewire and/or a guidewire and spring element assembly. In an exemplary embodiment, a stiffening element or the like such as a stylet can be located in or adjacent to the termination 860. By controlling the location of the stylet, such as by removing the stylet, the termination can then be caused to bend or otherwise flex. By way of example only and not by way of limitation, in an exemplary embodiment the termination has a memory, and the stylet or the like resists that memory so as to make the termination to be straight or to be curved for example. Upon removal of the stylet and/or guidewire or what have you, the termination will return to its relaxed state. Conversely, upon insertion of the guidewire and/or stylet etc., the termination will be forced away from its relaxed state.

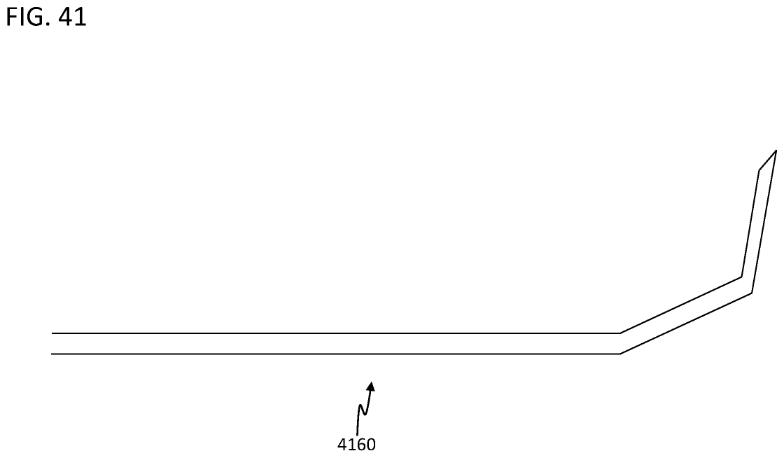

In some alternate embodiments, instead of portions of the termination (and/or conduits disclosed below (which can be microlumens) that bend or flex, articulating joints can be utilized. FIG. 41 presents an exemplary termination 4160 where there are two separate joints as shown. In an exemplary embodiment, there can be one, two, three, four, five, six, seven, eight or nine or more joints or any value or range of values therebetween.

In an exemplary embodiment, there can be one, two, three, four, five, six, seven, eight or nine or more sections of bending spaced by sections of non-bending or any value or range of values therebetween.

While many of the embodiments disclosed herein relate to terminations and/or conduits and/or scopes, etc., having constant outer diameters, in alternative embodiments, the diameters can be varied over the length of the pertinent component.

In an exemplary embodiment, the angle(s) of the bend/flexure can be controlled. In an exemplary embodiment, the termination can be controlled or otherwise be flexed over a range of angles, measured in a plane, of at least 5, 10, 15, 20, 25, 30, 35, 40, 45, 50, 55, 60, 65, 70, 75, 80, 85, 90, 95, 100, 105, 110, 115, 120, 125 or 130 degrees or any value or range of values therebetween in 1° increments.

In an exemplary embodiment, the termination can be a composite structure and/or an amalgamation different materials with one portion made out of a rigid structure and/or a rigid material and/or a material or structure that maintains rigidity through the process and another portion made out of the flexible material and/or has a structure that is flexible. In an exemplary embodiment, nitinol or some other material can be used, which maintains a geometry during a certain time and/or under certain circumstances (such as, for example, temperature), and then deformed. Thus, in an exemplary embodiment, section 864 can be made out of nitinol or other memory shape material, and the nitinol/termination 860 can be placed into a liquid ice and/or dry ice slurry, where section 864 achieves a relatively straight or a straight geometry, and then, after insertion through the tympanic membrane and entering the round window niche, as the termination warms (which could be a result of running warm irrigation fluid through the lumen of the termination and/or as a result of electrical current being run through a separate heating element (resistor) in the termination/next to the termination and/or via a conductive path from a remote heating device to the part that is to be heated, etc.) or run directly through nitinol to heat such up), section 864 bends around the top of the round window niche, such that the end/point 866 of the termination 860 is now aligned or otherwise pointed towards the round window. Further insertion of the ear system endoscope into the ear now results in the tip 866 of the termination being driven into and thus through the round window niche, and then into the duct of the cochlea, where the therapeutic substance can then be transferred through the termination and into the duct, and thus into the cochlea.

In an exemplary embodiment, the termination is a 27 gauge termination.

In an exemplary embodiment, an inner diameter of the lumen of the termination and/or of the various conduits detailed herein is less than or equal to 500, 450, 400, 350, 300, 250, 200, 175, 150, 140, 130, 120, 110, 100, 90, 80, 70, 65, 60, 55, 50, 45, 40, 35, 30, 25, 20, 15 or 10 micrometers or any value or range of values therebetween in 1 μm increments.

In an exemplary embodiment, the outer diameter of the termination and/or the outer diameter of the various conduits and/or optical cable and/or the drill bits detailed herein is less than or equal to 1000, 900, 800, 700, 600 500, 450, 400, 350, 300, 250, 200, 175, 150, 140, 130, 120, 110, 100, 90, 80, 70, 65, 60, 55, 50, 45, or 40 micrometers or any value or range of values therebetween in 1 μm increments. In an exemplary embodiment, the aforementioned values are associated with the tip of the tool that delivers the actual payload. Still, it is noted that in at least some exemplary embodiments, the terminations of the endoscopes can be somewhat larger, such as by way of example, having an outer diameter of less than or equal to 5, 4.5, 4, the 3.5, 3.25, 3, 2.75, 2.5, 2.25, or 2 mm point or any value or range of values therebetween in 0.01 mm increments.

While the embodiments detailed herein are for the most part directed towards the utilization of the tools disclosed herein to access the cochlea for the purposes of providing a therapeutic substance therein, some embodiments are directed toward utilizing the teachings detailed herein to implant a cochlear implant electrode array into the cochlea. Accordingly, in an exemplary embodiment, there are methods that utilize one or more or all of the teachings detailed herein, modified albeit in a utilitarian manner, to implant a cochlear implant electrode array into the cochlea. It is also noted that in an exemplary embodiment, there are methods that utilize one or more or all of the teachings detailed herein, modified albeit any of the solitary manner, to implant other types of stimulators, such as stimulators for balance and/or to address tinnitus. Thus, any disclosure herein of utilizing the teachings herein with respect to a therapeutic substance corresponds to an alternate disclosure of utilizing those teachings to implant a cochlear implant electrode array and/or a balance implant and/or a tinnitus treatment device and/or an intracochlear mechanical stimulator. By way of example only and not by way of limitation, in an exemplary embodiment, element 300 of FIGS. 4A to 4E can correspond to the terminations and/or the other conduits or the like disclosed herein.

It is noted that in at least some exemplary embodiments, the teachings detailed herein are directed to specific devices that have a limited utility or use. For example, the ear system endoscope is a device utilized for the ear system, and not for anything else. In this regard, in an exemplary embodiment, the various teachings detailed herein can be directed to FDA approved products for use in a given field for a given purpose and for nothing else.

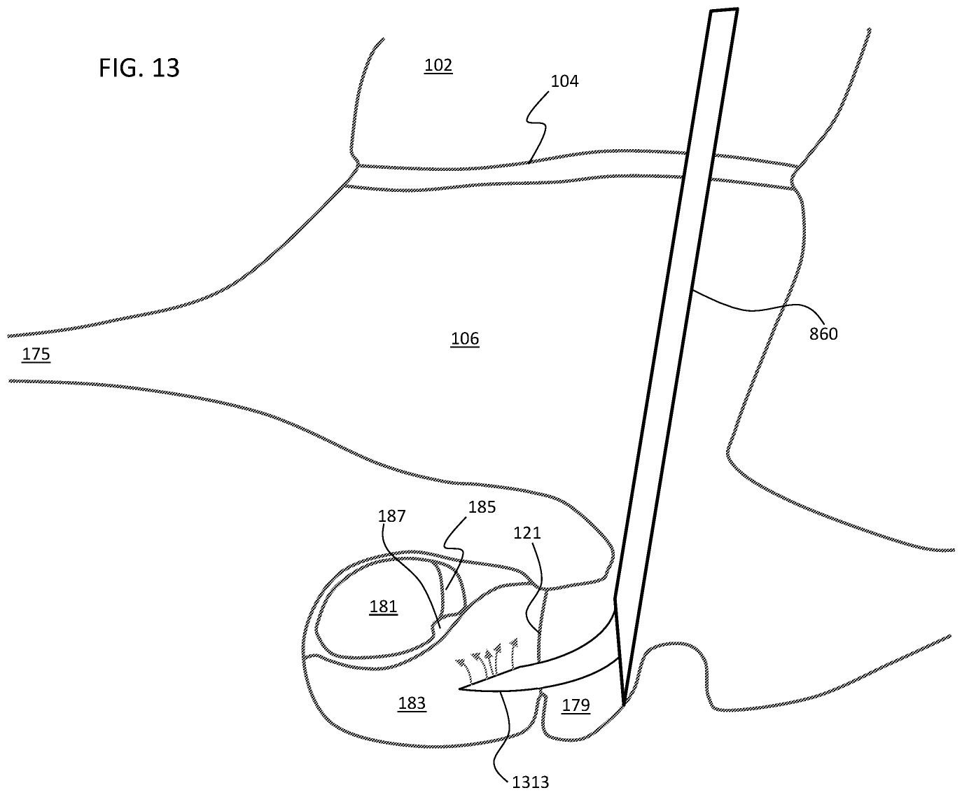

FIG. 13 represents a conceptual example of how the inner ducts of the cochlea can be accessed utilizing an exemplary ear system endoscope. Briefly, shown is a cross-section of the outer middle and inner ear, where the scala tympani 183 is shown below the scala vestibule 181. For frame of reference, also shown is the scala media 185 and the organ of Corti 187. Here, the ear system endoscope includes a secondary fluid conduit 1313 that is movable relative to the termination 860 in general, and can move inside the lumen of the termination 860 in particular. In this exemplary embodiment, the termination 860 is utilized to puncture through the tympanic membrane 1040 as shown. The termination 860 is then extended downward into the round window niche 179. In this exemplary embodiment, the tip of the termination 860 is utilized to provide a “brace” on the bone so as to steady the termination 860 in general, and the distal portion thereof in particular. The sharpness of the tip can be utilized to slightly “dig” into the bone. In some embodiments, this is not necessarily the case. For example, the ear system endoscope can be positioned so that the tip of the termination 860 is held proud relative to the surface of the bone. In any event, with the chamfer of the termination 860 facing the round window 121, upon the final placement of the termination 860 in or adjacent to the round window niche, the secondary fluid conduit 1313 can be extended out of the opening of the termination 860 such as by providing a force to a surgical device (or to an portion of the secondary fluid conduit 1313 that extends from the port 830 to the end of the termination 860) which is mechanically linked ultimately to the secondary fluid conduit 1313 through the surgical tool receptacle/port 830. This force drives the secondary fluid conduit 1313 out of the opening of the termination 860.

In some embodiments where there is a steerable tip in one or more planes (steerable over at least 5, 10, 20, 30, 40, 50, 60, 70, 80, 90, 100, 110, 120, 130 degrees or more—more on this below), and brace on a point on the bone or other body part having utilitarian value, one can also pivot more than 90, 135, 150, 180 degrees or more around the axis of the endoscope and or/termination, one can cover more than 10, 15, 20, 25, 30, 35 or 40 percent of a sphere space. This give utilitarian flexibility to cover a range of human anatomy variations associated with size and positioning of the round window and tympanic membrane.

In this exemplary embodiment, the secondary fluid conduit 1313 can be a metallic tube having an outer diameter that is smaller than the inner diameter of the termination 860, thus permitting the secondary fluid conduit 1313 to extend through and move relative to the termination 860. In an exemplary embodiment, the metallic tube can be made of a relatively flexible material or otherwise can be configured to flex or otherwise bend so that upon reaching the end of the termination 860 and/or otherwise exiting the termination 860, the secondary fluid conduit 1313 will move in a direction towards the round window 121. This can be accomplished by utilizing a material that has a memory shape that when in a relaxed state, the conduit 1313 bends away from the trajectory of the termination 860. That is, when the conduit 1313 is not restrained by the inner diameter of the termination 860, the conduit 1313 would coil or otherwise curve. As can be seen, the end of the conduit 1313 can have a chamfered end in a manner concomitant with the chamfered end of the termination 860, which chamfered end creates a sharp and that can enable the puncturing of the round window as seen.

While the embodiment just described utilizes a memory shape conduit 1313, in alternate embodiment, the bending of the conduit 1313 can be established by resistive methods. For example, in an exemplary embodiment, within the lumen of the termination 860, there can be a ramp like component sized and dimensioned so that when the conduit 1313 reaches the ramp, the ramp drives the conduit away from the trajectory (the longitudinal axis) of the termination 860, and thus towards the round window 121. In an exemplary embodiment, if the tip of the endoscope is steered by guidewires and/or spring, or by any other arrangement, or even by general positioning (e.g., in the case of the bent/flexed termination), one can point the tip at the round window and the bending of 1313 is achieved by following the curvature of the bent tip/flexed tip of the endoscope and then the conduit 1313 proceeds in a straight line/perpendicular to the tip of the scope. It is briefly noted that in at least some exemplary embodiments, the various steering and/or guidance and/or other navigation regimes and control of positioning regimes are discussed in terms of the termination and/or the conduit and/or other components of the endoscope. It is noted that any disclosure herein with respect to such associated with a given component, such as the termination, corresponds to an alternate disclosure of another embodiment where such is related to the other components detailed herein, such as the conduit that extends through the termination and/or the drill bits and/or the optical portions of the endoscope.

Upon puncturing the round window 121, the therapeutic substance can be delivered under pressure or by whatever transport regime that can have utilitarian value through the inner lumen of the conduit 1313, and thus into the scala tympani 183 as shown, where the therapeutic substance delivery is represented by the arrows extending from the chamfer of the conduit 1313. In an exemplary embodiment, the teachings detailed herein are implemented by taking into account pressures and/or speeds of delivery of the therapeutic substance. In an exemplary embodiment, the pressures and/or speeds (rate of flow, volume and/or mass etc.) etc., are controlled so as to avoid any deleterious effects associated with hearing and/or balance, etc. In an exemplary embodiment, the systems detailed herein are configured to automatically monitor and/or control the pressures and speeds, etc. In an exemplary embodiment, memory and/or lookup tables can be included with the system, and the monitored pressures and/or speeds can be compared to the lookup tables relating to safe and/or utilitarian pressures and/or speeds, and the pressures and/or speeds can be controlled based on those lookup tables. For example, if the pressure is too high, the system can be automatically adjusted so as to reduce the pressure. Alternatively, if the pressure is below a safety value, and there is utilitarian value with respect to increasing the pressure, the pressure can be automatically increased. Note that in some embodiments, the system detailed herein can be configured for manual adjustment as well. In some embodiments, automatic shutdown features exist in a scenario where there is a pressure and/or speed that is deemed too high or previously has been deemed too high, which meaning is executed automatically.

It is noted that while the embodiment of FIG. 13 is presented with respect to the conduit 1313 within the lumen of the termination 860, the general concept of FIG. 13 also can be utilized to represent the utilization of the flexible termination 860 as detailed above and/or other arrangements (e.g., where the end of the termination is curved).

Embodiments can be directed to steerable conduits 1313 and/or steerable terminations 860. This can be done by utilizing electrical currents and/or heating of various portions of the overall components. In an exemplary embodiment, the application of current can cause localized heating. In an exemplary embodiment, by utilizing different materials that have different electrical resistivity, parts of the conduit and/or termination will be heated to a different temperature, and thus the local expansion of the local portions can be controlled so as to steer the component. By way of example only and not by way of limitation, magneto restrictive material can be utilized. Various MEMS technology can be utilized and/or piezoelectric technologies can be utilized to steer the aforementioned noted components. Any technology that can enable the steering or otherwise the guidance of the components at issue that can enable the teachings detailed herein can be utilized in at least some exemplary embodiments. By way of example only and not by way of limitation, technology utilized for tiny internal medicine catheter and/or scope steering and/or guiding can be utilized. In an exemplary embodiment, the end of the conduit 1313 and/or the end of the termination 860 and/or the entirety of these components can be made out of the magnetic material, and after the working portions are located within the round window niche for example, a magnet can be brought proximate to the head of the patient, which magnet is powerful enough to “pull” the tip of the conduit and/or the termination “around” the top portion of the round window niche/the overhang so as to point the tip at the round window 121, afterwards the conduit and/or the termination can be pushed further into the patient, thus piercing or otherwise incising the round window 121.

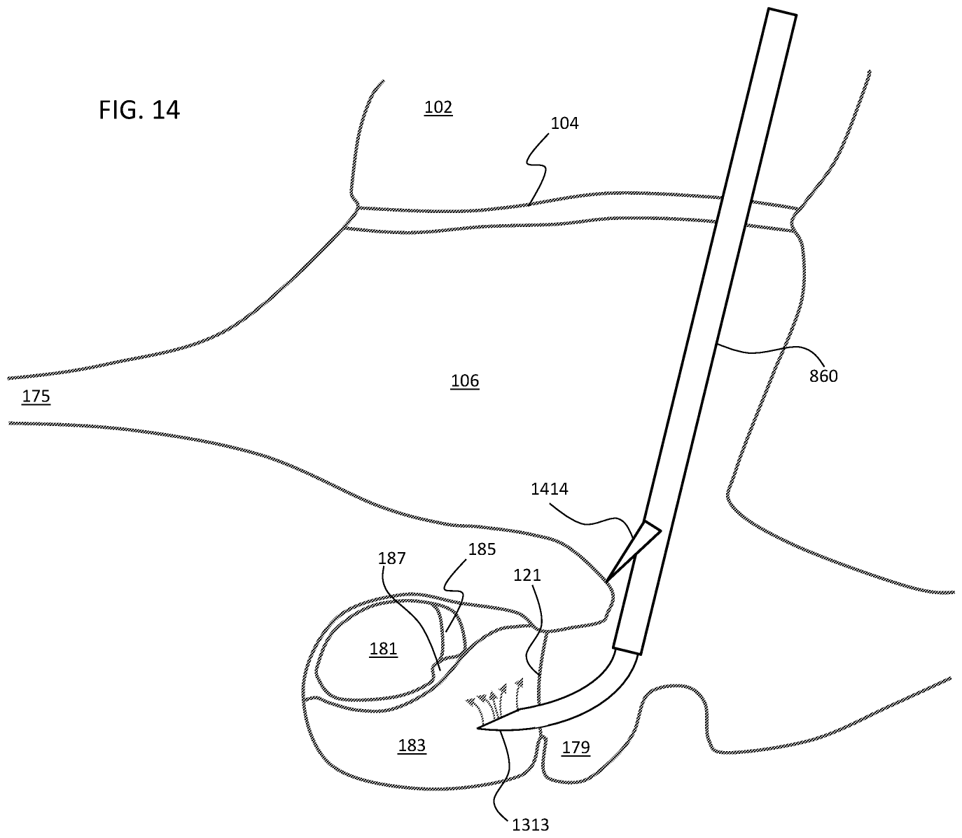

FIG. 14 presents an alternate exemplary embodiment where a niche hook 1414 is attached to the termination 860. In this exemplary embodiment, the niche hook 1414 is deployable, which includes self deployable, from the lateral side of the termination 860, wherein in the retracted state, it is easier to pass the termination 860 through the tympanic membrane 104. After passing through the tympanic membrane 104, the hook 1414 can extend outward to the positions shown so that the hook 1414 can grab onto the overhang of the round window niche as shown, thus halting further insertion of the termination 860. The arrangement can be sized and dimensioned so as to hold the end of the termination 860 a desired distance from the bottom of the round window niche so as to provide room for the deployment of the conduit 1313.

In an exemplary embodiment, a shroud can be located distal of the niche hook 1414. This can be an angled portion that can be part of the termination 860 that will gently expand the hole through the tympanic membrane so as to provide room for the hook 1414 in the retracted state to pass therethrough. The niche hook 1414 can be deployable utilizing a magnet for example (the hook could be magnetic, or a component that holds the hook in the retracted steak of the magnetic, etc.) or can be actuated to the deployed state utilizing an electrical actuator or some other actuator (a hydraulic actuator).

Thus, as seen above, embodiments can include a device that includes a component configured to limit and/or frustrate further insertion of a distal end of the device into a head of a human beyond that which would be the case in the absence of the component. In an exemplary embodiment, the component can be a promontory hook and/or a foot, and/or can be a component located in the ear canal.

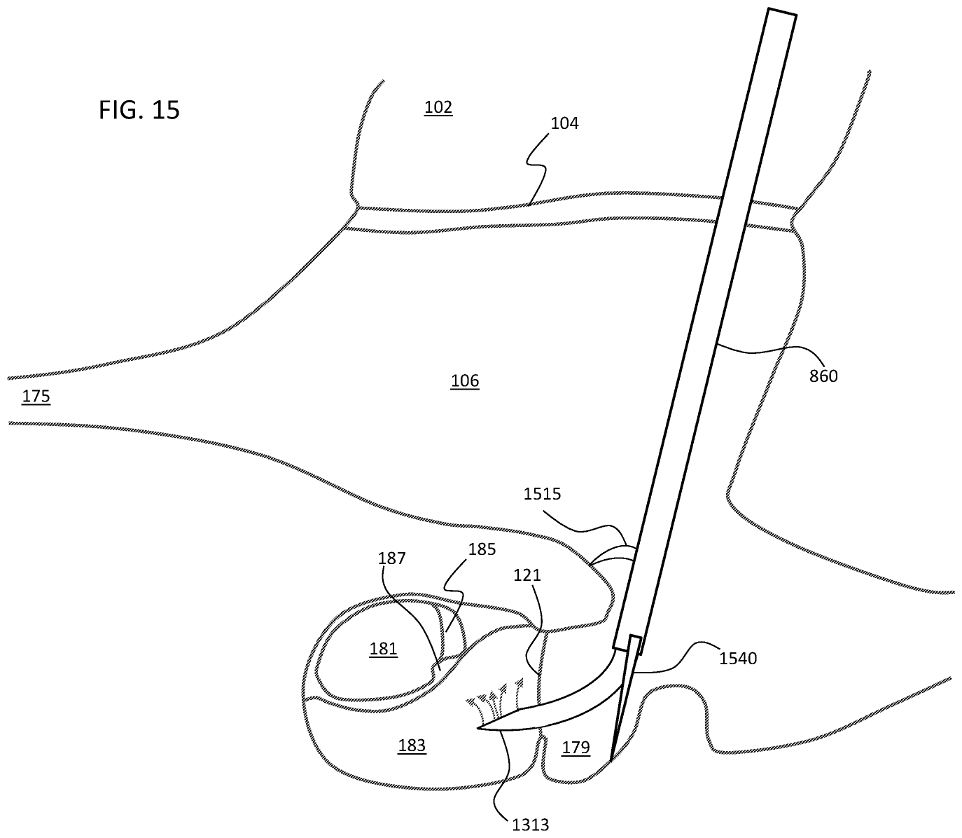

The embodiment shown in FIG. 14 has a blunt end of the termination 860 as seen. In this exemplary embodiment, another tool can be utilized to make the initial hole through the tympanic membrane 104. That said, in some embodiments, instead of a hole per se, the tympanic membrane is “flapped” (a portion of the tympanic membrane is cut so that it will flap down (or up) to provide room for the termination 860. In some exemplary embodiments, a Coring needle and/or a non-coring needle is used. In an embodiment, the component(s) can be extended through a grommet in the tympanic membrane. With respect to embodiments utilizing a flapping to obtain access to the middle ear from the outer ear through the tympanic membrane, a “fixed” niche hook 1515, as seen in FIG. 15, can be utilized instead of a deployable hook. That said, it is possible that a fixed niche hook could be utilized in embodiments that utilize a piercing of the tympanic membrane. FIG. 15 also shows an exemplary niche stop 1540 as seen. This component can be fixed or deployable and can be utilized to stop the distal tip of the termination 860 at the desired location.

It is noted that some embodiments specifically do not flap the tympanic membrane. In this regard, some embodiments only puncture the tympanic membrane, whereas flapping the tympanic membrane involves cutting the tympanic membrane. By rough analogy, a syringe does not cut the skin but instead punctures the skin whereas a surgical scalpel cuts the skin.

The niche stop 1540 can also represent a cutting tool that can be utilized to cut through the tympanic membrane, instead of utilizing the chamfered end of the termination 860 with respect to the embodiment of FIG. 13 for example. In this regard, component 1540 can be a sharp surgical scalpel and/or a miniature surgical scalpel that can be utilized to carefully cut an opening in the tympanic membrane, through which the termination 860 can be extended through to reach the round window niche. The surgical sample can also be utilized to position the termination 860, by utilizing the sharp edge at the end to dig into the bone a bit, which will prevent the termination 860 from moving in the lateral directions.

In view of the above, it can be seen that in an exemplary embodiment, there is a device, comprising an ear system endoscope configured to incise through tissue to reach duct(s) of an inner ear of a human and to deliver a therapeutic substance to the duct(s) through a resulting incision. As seen above, the ducts can be the scala tympani or the scala vestibule of the cochlea, for example. As seen above, in an exemplary embodiment, the device includes a sharp termination configured to incise through tissue, such as the tympanic membrane, and in some embodiments, the round window, to reach the duct(s), the termination having a lumen therein through which the therapeutic substance can be delivered to the duct(s).

Still further, in an exemplary embodiment, the device can include a drill bit configured to incise (here, drill) through tissue (here, the promontory bone of the cochlea) to reach the duct(s), the drill bit having a lumen therein through which the therapeutic substance can be delivered to the duct(s). In an exemplary embodiment, the device can include one or more components configured to fix a distal end of the device in or proximate a round window niche of the lumen. This can correspond to element 1540 noted above for example, element 1515 noted above by way of example only and not by way of limitation. In an exemplary embodiment, this component that is configured to fix the distal end is a separate component from a sharp end of the termination of the device.

Corollary to this is that in some embodiments, the device includes a conduit with a sharp end configured to pierce a round window of the human to reach the duct(s) of the inner ear.

In some embodiments, the device is a hand held device configured reach the duct(s) via a route that extends through a tympanic membrane and then across a middle ear to at least one of a promontory within a middle ear of the human or a round window niche of the human. And in some embodiments, the device includes a light capture apparatus configured to capture light emanating from a distal end of the tool and a display in communication with the light capture apparatus, configured to display an image captured by the light capture apparatus, wherein the device is a hand held device.

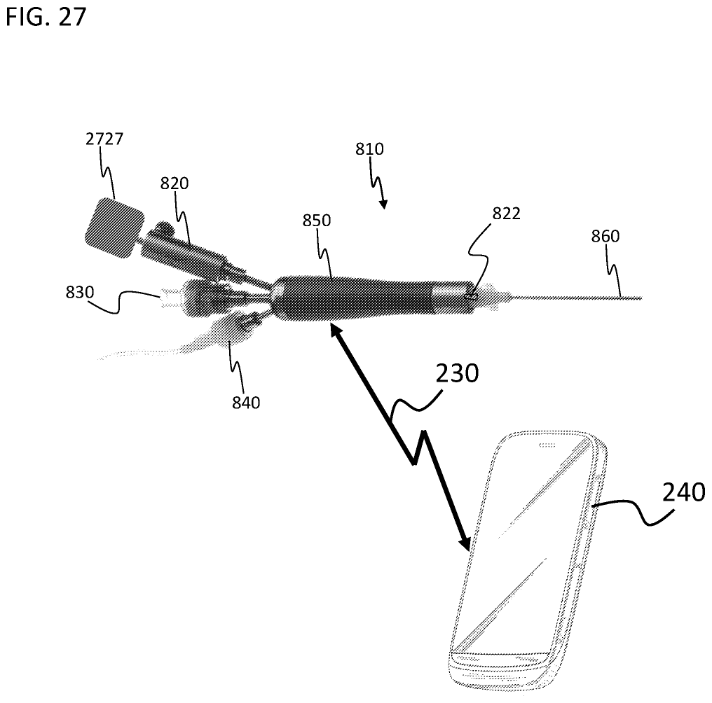

In an exemplary embodiment, the display of the ear system endoscope is hard mounted to the ear system endoscope. In an exemplary embodiment, the display of the ear system endoscope is mounted to the remaining components such that any movement of the proximal end of the termination in a straight line vector in the Cartesian coordinates corresponds to a one-to-one movement of the display also in a straight line vector in the Cartesian coordinates. In an exemplary embodiment, the display of the ear system endoscope is mounted to the remaining components such that any rotational movement of the proximal end of the termination about the longitudinal axis of the termination corresponds to a one-to-one rotation of the display. All of this as opposed to the arrangement of FIG. 11. In an exemplary embodiment, the device includes a light capture apparatus configured to capture light emanating from a distal end of the tool, and device includes a display in communication with the light capture apparatus, configured to display an image captured by the light capture apparatus, wherein the display is at least indirectly hard mounted to a base of the ear system endoscope, and wherein the termination is at least indirectly mounted to the base. This as opposed to, for example, the embodiment of FIG. 11, where the display of the laptop computer is not hard mounted even indirectly, to the base 850. This also as opposed to, for example, the embodiment of FIG. 27, where, for example, an electrical cable is utilized to connect the endoscope to the handheld device 240, which electrical cable is flexible akin to most electrical cables utilized to, for example, recharge cell phones, etc. It is briefly noted that a system of joints and/or flexible supports that can enable local repositioning of the display still constitutes hard mounted. This as opposed to, for example, a mere table or the like connected to the display. If the connecting components can be configured to maintain a position of the display relative to the base, such is hard mounted. In an exemplary embodiment, the base 850 can be considered a main body of the endoscope, as opposed to, for example, component 820 and/or 840, etc.

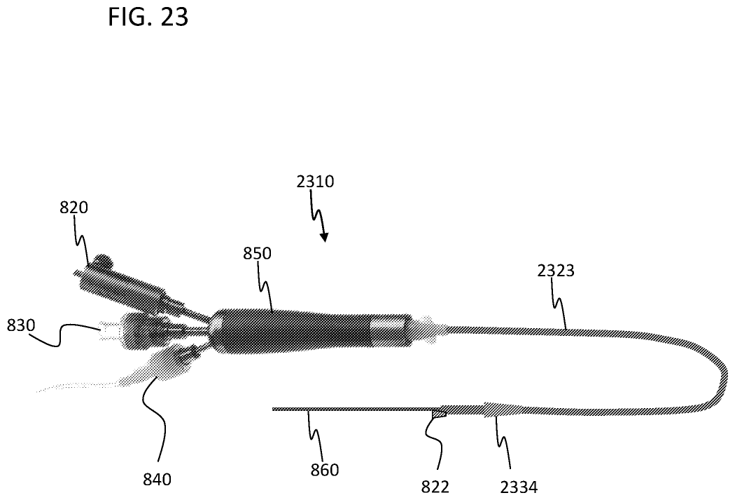

By handheld device, it is meant that the device can be held easily in a person's hand (or two hands, such as might be the case with the embodiment of FIG. 23) and is designed for primary use along those lines. This as opposed to the embodiment of FIG. 11 for example, even if the laptop could be held in a person's hand. In the end, the arrangement of FIG. 11 is not designed to be a handheld system.

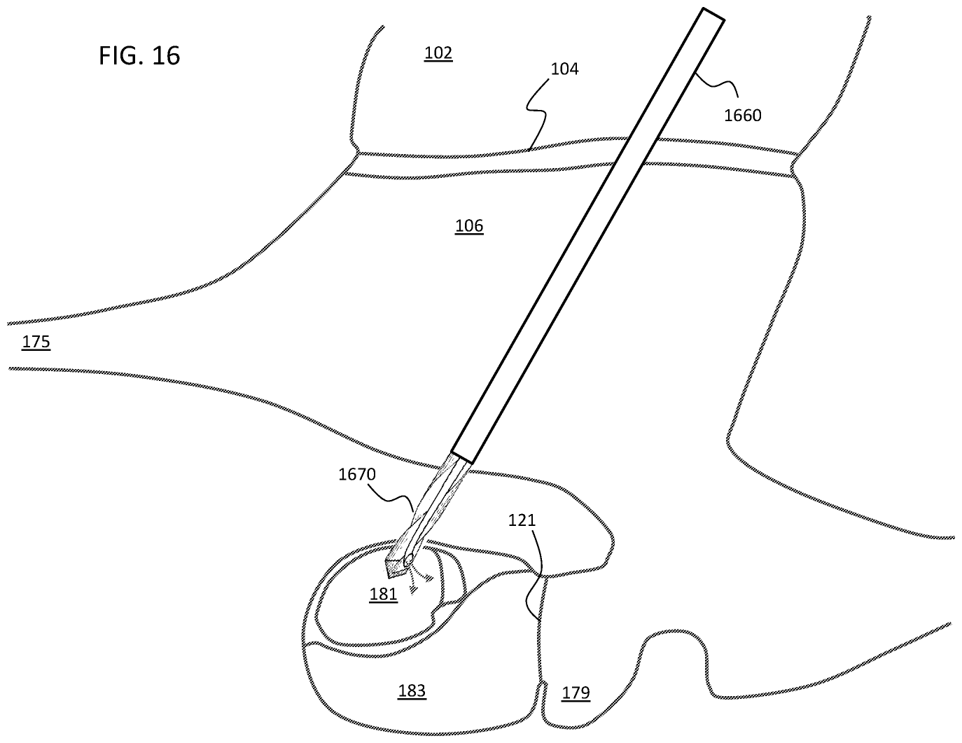

FIG. 16 depicts another exemplary embodiment for accessing the inner ducts of the cochlea. Here, the termination 1660 of the ear system endoscope includes a drill bit 1670. In an exemplary embodiment, the entire termination 1660 or at least the portions extending through the tympanic membrane to the distal end thereof is configured to be rotated at a speed that can enable the drill bit 670 to be used to perform a cochleostomy as shown in FIG. 16. In some embodiments, it is only the drill bit that is rotated, and in other embodiments, it is the entire or most of the termination 1660. In an exemplary embodiment, the drill bit can be part of an assembly or structure that extends through the lumen of the termination 860. That is, in an exemplary embodiment, the termination 860 above can be utilized to pierce the tympanic membrane 104, and then the tip of the termination 860 can be moved to the promontory, and in an exemplary embodiment, the sharp tip can be utilized to stabilize the termination 860, and then the drill bit apparatus can be extended through the lumen of the termination 860, and then upon reaching the tissue of the promontory of the cochlea, the drill bit can be turned so as to drill into the promontory and thus reach the duct of the cochlea as shown in FIG. 16. That said, in an alternate embodiment, the termination 1660 is utilized to puncture the tympanic membrane and to drill through the promontory to reach the duct of the cochlea. In an exemplary embodiment, the tip of the drill bit is sufficiently sharp so as to pierce the tympanic membrane without turning the drill bit, while in other embodiments, the drill bit is turned to pierce the tympanic membrane.

Accordingly, in an exemplary embodiment, the ear system endoscope can have a drill motor associated there with to turn the termination 1660. Accordingly, in an exemplary embodiment, the surgical port 830 can receive a drill bit apparatus that extends from a drill motor of a surgical tool located outside the ear system endoscope, which drill bit apparatus extends through the conduit of the termination 860 and wherein the systems are configured so that the drill bit can extend outside of and pass the end of the termination 860 so that drilling can commence.

In view of the above, it can be seen that in an exemplary embodiment, there is the action of turning a drill bit having a component of the drill extending through the tympanic membrane. In an exemplary embodiment, the drill is rotated at more than or equal to 300, 400, 500, 600, 700, 800, 900, 1000, 1100, 1200, 1300, 1400, 1500, 1600, 1700, 1800, 1900, 2000, 2250, 2500, or 3000 RMP or more, or any value or range of values therebetween in 10 RPM increments and this can be done while the drill extends the tympanic membrane,

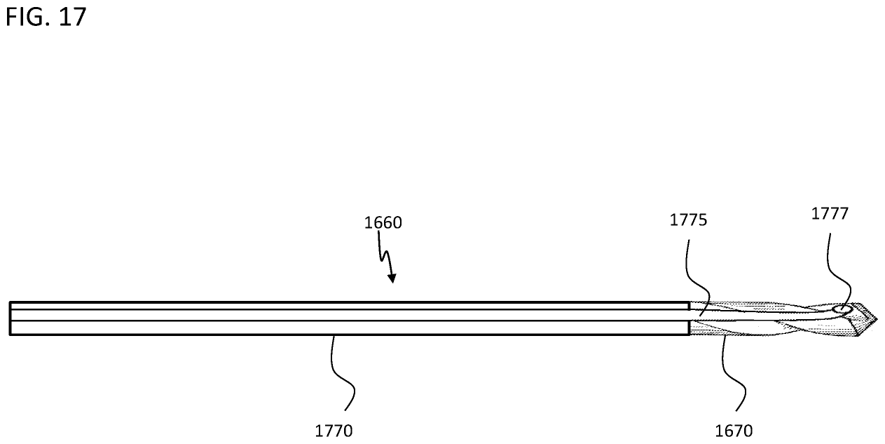

FIG. 17 depicts a cross-section of the termination 1660 of the embodiment of FIG. 16. As seen, there is a conduit 1775 extending through the termination, terminating at an orifice 1777. In an exemplary embodiment, the conduit and the orifice are utilized to provide irrigation and/or cooling fluid during the action of drilling to the ducts of the cochlea. In addition, or alternatively, the conduit and the orifice can be utilized to deliver a therapeutic substance into the duct(s) of the cochlea upon drilling through to the duct(s) of the cochlea. This is represented by the curved arrows emanating from the orifice shown in FIG. 16.

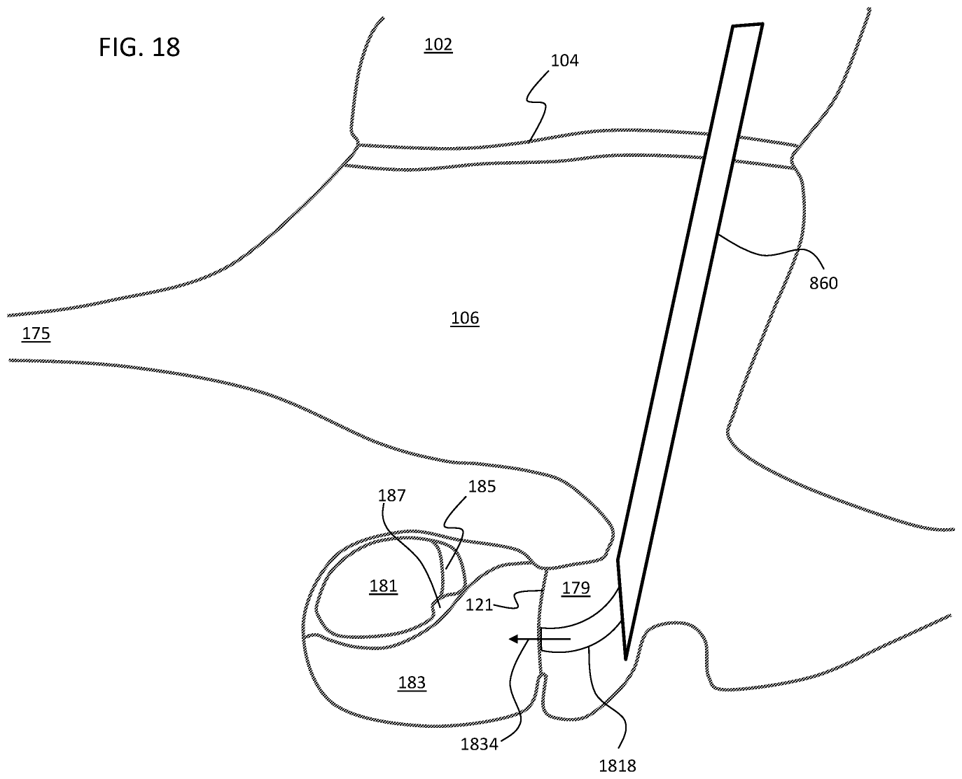

FIG. 18 presents an alternate exemplary embodiment of providing therapeutic substance to the interior of the cochlea. Here, a tube 1818 can be extended through the lumen of the termination 860, which to can be controlled/steered to the round window 121 or otherwise automatically with the extends towards the round window 121 if the termination 860 is oriented in the proper direction. In any event, the tube 1818 extends towards the round window to be in contact or otherwise in close proximity thereto. Then, a jet of fluid represented by arrow 1834 is ejected from the end of the tube 1818, which jet cuts through the round window 121 so that an opening in the round window will result. In an exemplary embodiment, this jet can be a therapeutic substance. The therapeutic substance can continue to be delivered through this opening into the cochlea so as to impart the therapeutic substance into the ducts of the cochlea. Alternatively, in an exemplary embodiment, an initial cutting agent can be utilized as the fluid to open the round window, and then the therapeutic substance can be provided. In an exemplary embodiment, the relative pressures between the initial cutting agent and the therapeutic substance can be varied, and in an exemplary embodiment, even when the same substance is utilized to cut through the round window and be delivered into the round window, the pressure can be varied upon the completion of cutting, as a pressure utilized for cutting may not be needed for delivery the therapeutic substance. In an exemplary embodiment, the working end of the tube 1818 can be configured to create a seal or a partial seal against the round window.

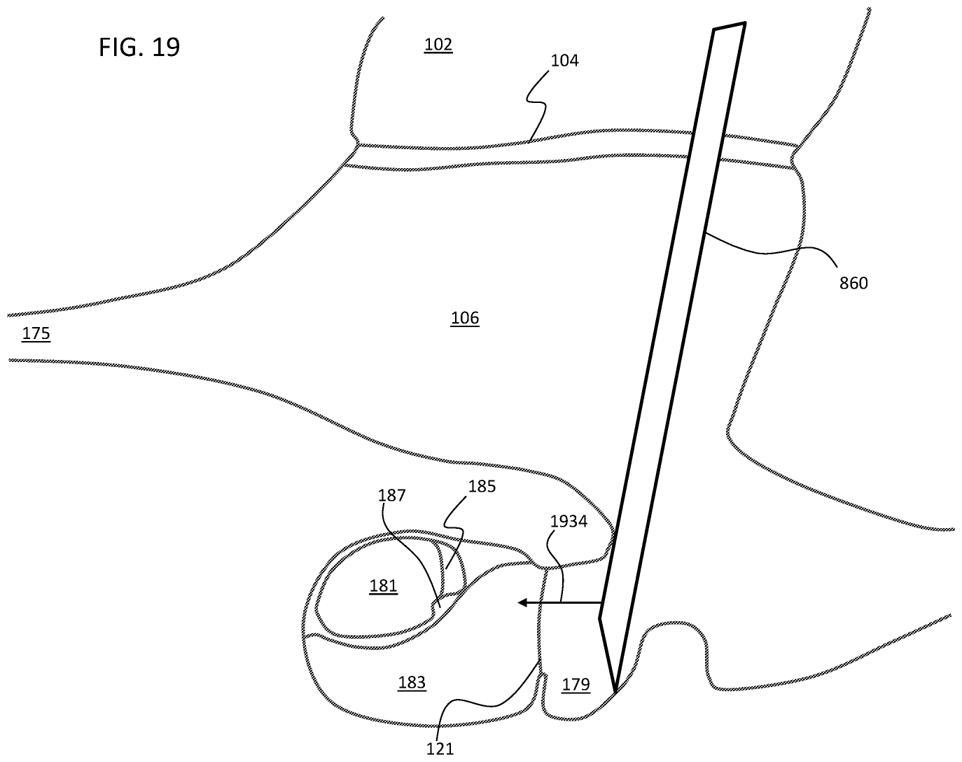

FIG. 19 presents an alternate exemplary embodiment where the jet 1934 extends from an orifice on the side of the termination 860. Here, there is no component that extends from the termination to the round window. Instead, the jet is sufficiently controlled and defined so that he can cut through the round window 121. Again, the jet of fluid can be the therapeutic substance or a cutting agent (which could be water). In some embodiments, the principle of operation can be the same as the embodiment of FIG. 18 detailed above.

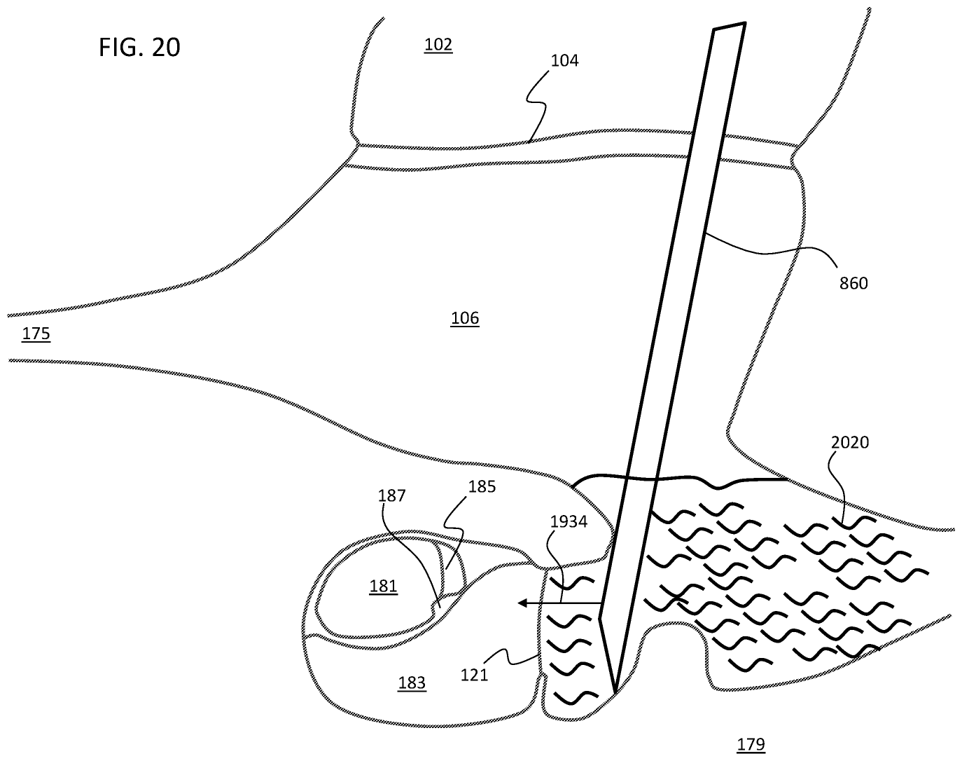

The embodiment of FIG. 20 presents an alternate exemplary embodiment, where the jet 1934 is utilized to perforate the round window 121, and then a therapeutic substances delivered through the jet orifice or through the end of the lumen of the termination 860, and the portions of the middle ear proximate the round window or otherwise the round window niche is filled with (or a sufficient amount is provided therein) therapeutic substance 2020 as seen. The therapeutic substance 2020 can flow through the opening in the round window 121 over time (as opposed to diffusing through the round window 121). It is noted that this technique can be utilized with other embodiments that do not utilize a jet to cut through the round window, but instead use some other device utilized to create an opening in the round window. Again, this is all distinct from diffusion or leaching through the membrane of the round window. Here, there is an opening that is created, an artificial opening, that is created, that is utilized to move the therapeutic substance from outside the cochlea to inside the cochlea.

While FIG. 19 is depicted as utilizing a jet 1934 to establish a passage through the round window 121, it is noted that a laser beam can be utilized instead. In this regard, element 1934 of FIG. 19 can instead represent a laser beam that is generated by the ear system endoscope in which is directed at an angle that is normal or oblique to the longitudinal axis as shown. In an exemplary embodiment, the direction of the laser beam can be controlled or otherwise steered by the user while maintaining a fixed orientation/position of the termination 860 (as it the case with all of the detailed steering and/or the control detailed herein). In an exemplary embodiment, this can be achieved via the utilization of fiber optics etc. In an exemplary embodiment, a laser or the like or some other device for that matter, such as a micro pin that can be moved over various locations, can be utilized to perforate or otherwise provide multiple preparations in the round window, and then the fluid delivery apparatus can be brought into create a seal and then deliver the therapeutic substance through the perforations. In this regard, the perforations can correspond to incising, providing that there is a definitive opening. Accordingly, in an exemplary embodiment, the conduit 1313 may not necessarily have a sharp end, but instead can have a and that has a ceiling feature, such as a silicon lip or some other feature, so that the conduit 1313 can create somewhat of a pressure chamber over the perforation, and then due to the increased pressure resulting from the therapeutic substance flow, the therapeutic substance will be “pushed” through the preparation and thus into the ducts of the cochlea.

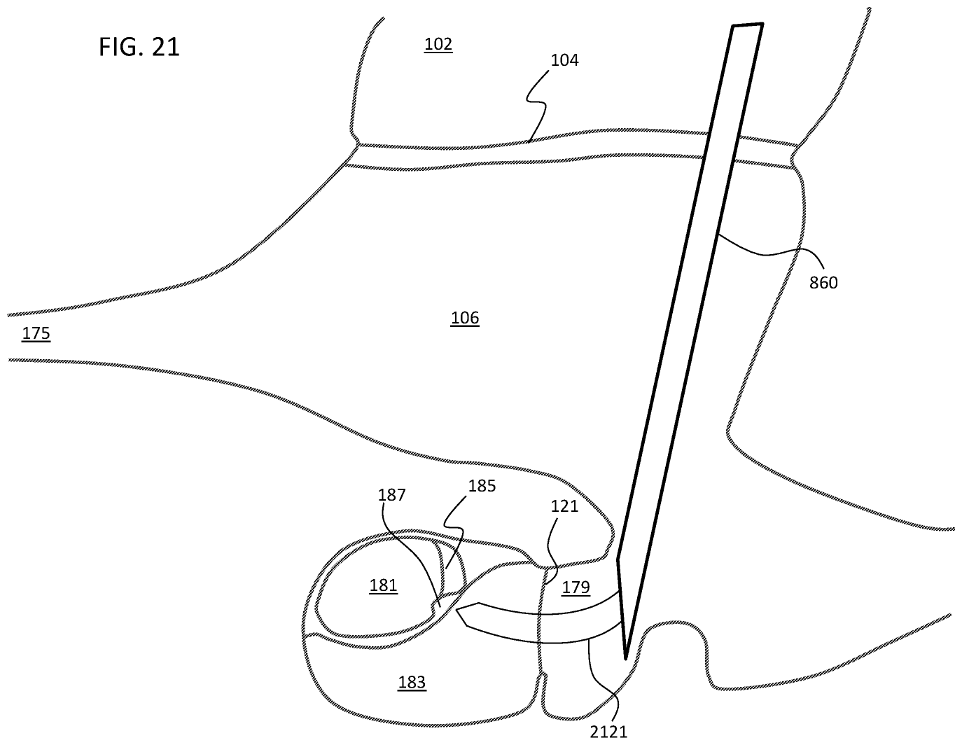

FIG. 21 presents another exemplary embodiment where a solid or a semisolid therapeutic substance 2121 is ejected through the end of the termination 860. This solid or semisolid therapeutic substance 2121 is sufficiently hard so that it can be utilized to pierce the round window 121. The end of the material 2121 can be formed so as to have a relatively sharp portion so as to pierce through the round window 121. The material 2121 can be then broken off so that the portions inside the cochlea remain there and will be dissolved by the perilymph so as to treat the cochlea. In an exemplary embodiment, the therapeutic substance can simply be delivered without breaking and when the “end” of the material 2121 is reached/is ejected from the end of the termination 860, the termination can be removed and a portion of the material 2121 can extend through the round window. Over time, the portion extending through the round window will fall off either by breakage or because the portions within the cochlea will diffuse perilymph to the boundary extending through the round window.

It is noted that the concept of utilizing a solid or semisolid therapeutic substance is also applicable to the embodiment of FIG. 16. Indeed, in an exemplary embodiment, instead of the curved conduit proximate the orifice 1777 shown in FIG. 17, which may (or may not) interfere with movement of a solid substance, a more straight or a straight conduit can be utilized. A cylinder of the therapeutic substance can be forced through the conduit 1775 and out the orifice 1777 and thus into the cochlea.

In some embodiments, the therapeutic substance is contained in a solid drug body such that when wetted, elute therapeutic substance into the environment. The solid drug body could be a reservoir that could be wholly or partially exposed to fluid, and could be remote from the target of interest but fluidically connected. That is, the reservoir of therapeutic substance that is located outside the head and in communication with the ear system through the tool port can be a reservoir that includes or otherwise is the therapeutic substance in solid form, and a wetting agent can be provided so as to elute the therapeutic substance, which elution will travel down the channel to the target.

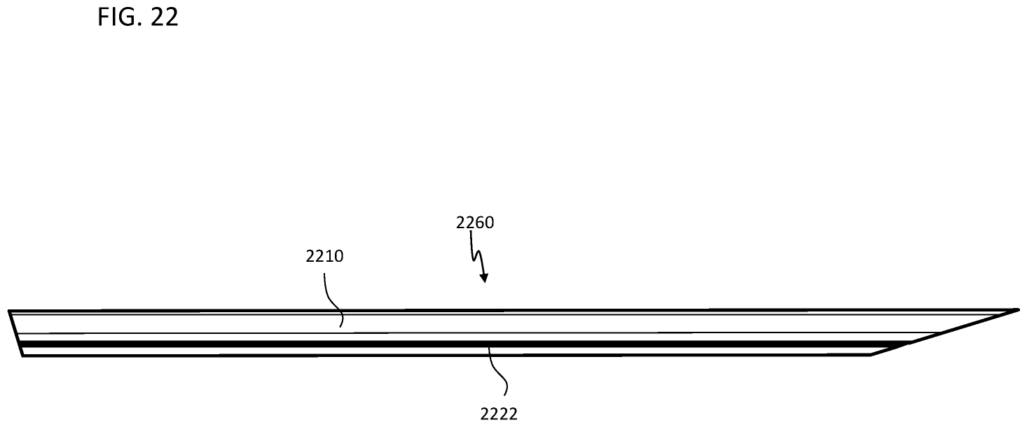

Some additional exemplary alternative configurations will now be briefly described. FIG. 22 presents an exemplary termination 2260 that includes a lumen 2210 configured for the movement of a therapeutic substance and/or of the various surgical devices detailed herein therethrough. Also included in the termination 860 is a fiber-optic channel 2222. This can be an integrated fiber-optic device or can be a tube through which a fiber-optic cable can moved so that light can be captured at the sharp end/distal end of the termination 2260. This can provide a surgeon or other healthcare professional or other user a view at the distal end of the ear system endoscope. In an alternate exemplary embodiment, the fiber-optic channel 2222 can be located outside the structure that establishes the lumen 2210. Any arrangement that can enable the teachings detailed herein can be utilized in some embodiments.

FIG. 23 depicts an exemplary ear system endoscope 2310 that utilizes a flexible cable 2323 to extend the working and of the endoscope away from the base 850 as shown. Here, a mounting 2334 which is sized and dimensioned to be held with an index finger and thumb or with a surgical tweezers or the like supports the termination 860 as shown. Also shown is a light capture apparatus 822. This can have utilitarian value with respect to allowing a surgeon or a healthcare professional freedom to manipulate only be working portions of the ear system endoscope as opposed to having to manipulate the entire ear system endoscope.

Embodiments above have concentrated on the utilization of an ear system endoscope that utilizes the ear canal in general, and a puncture through the tympanic membrane in particular, to reach the promontory and/or the round window niche. Of course, as noted above, in some other embodiments, a tympanic membrane flap is created so as to access the middle ear. It is also noted that in some exemplary embodiments, the middle ear in general, and the promontory in the round window niche and the oval window in particular, can be accessed through a more traditional surgery where a surgeon cuts into skin behind the pinna and then excavates bone/removes bone to reach the middle ear (a traditional method implemented to implant a cochlear implant for example). After this, for example, a hole can be drilled to the promontory in the case of a cochleostomy, and/or the round window can be punctured, etc. (such enable a trajectory that is more of a direct shot from the outside of the head to the round window for example). Any method of reaching the pertinent tissue that can enable the teachings detailed herein can be utilized in some exemplary embodiments, such as, for example, the etympanomeatal flap approach. Of course, as noted above, a tympanomeatal flap approach can be utilized as well.

It is noted that frequently, the word “surgery” is used herein. In some embodiments, the teachings detailed herein are applicable in procedures that might not be considered surgery per se. By way of example only and not by way of limitation, in the case of standard of care today for Meniere's disease, an intratympanic steroid injection is sometimes provided to a patient. In some instances, this is done in consultation rooms, and may not be considered surgical. Accordingly, any disclosure herein referencing a surgical procedure corresponds to a disclosure of an alternate embodiment that is something other than a surgical procedure, such as a procedure analogous to that just detailed which may be perhaps a “consultation” procedure. In an exemplary embodiment, the teachings detailed herein, at least some of them can be executed by a registered nurse and/or a nurse practitioner, and thus might not necessarily be considered surgery per se.

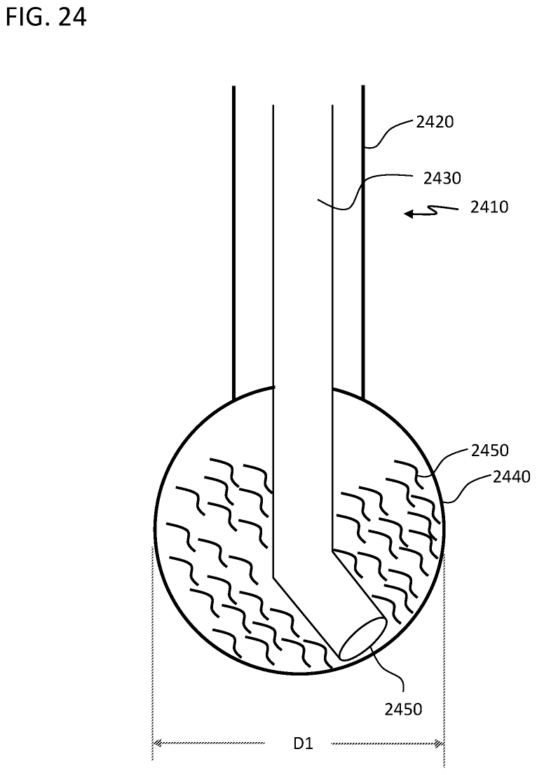

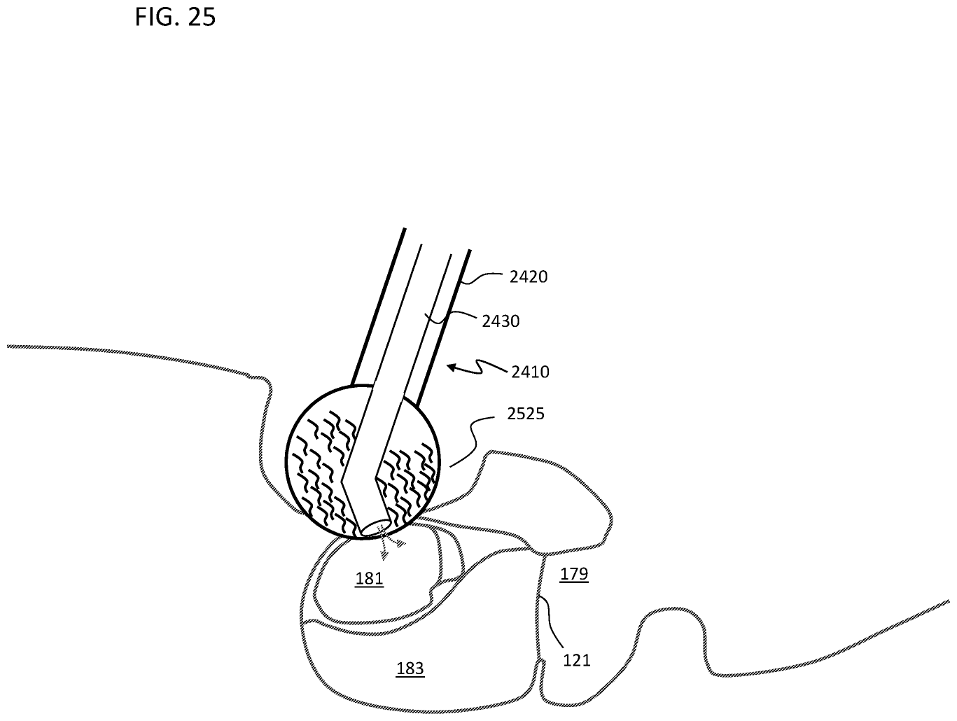

FIG. 24 presents an exemplary drill bit 2410 that can have utilitarian value with respect to the teachings detailed herein. This drill bit can be located at the end of the ear system endoscope and/or can be utilized separately from the ear system endoscope. Drill bit 2410 includes a shank 2420 through which a lumen 2430 extends to and orifice 2450 at the surface of the working end 2440 of the drill bit. Drill bit 2410 is a spherical drill bit, which includes a roughened surface 2450, which roughened surface is utilized to remove bone at the promontory for example so as to form a cochleostomy. In an exemplary embodiment, the roughened surface is established by a diamond arrangement. In this regard, the drill bit can be a diamond tip drill bit. FIG. 25 depicts utilization of the drill bit 2410 to form an excavation 2525 at the promontory of the cochlea, which excavation extends into the duct 181 of the cochlea as seen. In this exemplary embodiment, the therapeutic substance can be delivered through the conduit 2430 upon reaching the duct 181 of the cochlea. By way of example only and not by way of limitation, the orifice 2450 is located at a position that will not interfere with the overall drilling the features of the drill bit 2410, and then, upon the completion of drilling, the drill bit can be stopped, and the therapeutic substance can be channeled into the cochlea as shown, providing that the orifice 2450 is positioned properly. Such verification can be established utilizing the optics of an endoscope, such as the optics of an endoscope of which the drill bit 2410 is a part, or by eyeballing the arrangement if the more invasive surgical procedure is utilized to reach the promontory. It is noted that in some embodiments, the drill bit 2410 is sized and dimensioned so as to fit through a puncture through the tympanic membrane, while in other embodiments, a tympanic membrane flap is established so as to reach the promontory with the end of the drill bit 2410.

As can be seen in FIG. 25, the method of drilling through the promontory is such that the upper portions of the promontory experience more mass removal of tissue/bone than the lower portions. Indeed, as can be seen, only a small portion of the is utilized to working end of the drill bit is utilized to ultimately break through to the duct of the cochlea. By utilizing only a fraction of the overall diameter of the drill bit, the size of the breakthrough can be limited to that which is only needed. By way of example only and not by way of limitation, with respect to the maximum diameter of the drill bit and/or with respect to the maximum diameter D1 taken on a plane normal to the longitudinal axis of the drill bit is shown in FIG. 24, the maximum diameter of the breakthrough as measured at the interior wall of the duct 181 is no greater than or equal to 80, 75, 70, 65, 60, 55, 50, 45, 40, 35, 30, 25, 20, 15, 10 or 5% or any value or range of values therebetween in 1% increments (e.g., 61 percent, 33 percent, 77 to 52 percent, etc.) of the aforementioned maximum diameter(s) of the drill bit 2410.

In an exemplary embodiment, D1 is less than or equal to 1300, 1200, 1100, 1000, 900, 800, 700, 600 500, 450, 400, 350, 300, 250, 200, 175, 150, 140, 130, 120, 110, 100, 90, 80, 70, 65, 60, 55, 50, 45, or 40 micrometers or any value or range of values therebetween in 1 μm increments.

As noted above, embodiments can include a display or otherwise an image representation apparatus that can convey an image to a user of the ear system endoscope associated with light emanating from the distal end of the tool. This can have utilitarian value with respect to imaging the area around and at and of the tympanic membrane, and also imaging the area around the round window niche and/or promontory and the area thereof so as to provide a visual indication of the trajectory and/or the location of the distal portions of the ear system endoscope while the ear system endoscope is being inserted into the head of the human. By presenting an image to the user, the user can make adjustments to the trajectory and where the location of the various components of the ear system endoscope so as to guide the ear system endoscope to the desired location associated with the interior of the human.

In an exemplary embodiment, there is a data processing system that can at least provide an indicia to the user of the ear system endoscope or any other tool for that matter utilized to access the inner ear (at least some of the exemplary embodiments herein are directed towards tools that are not specifically an ear system endoscope per se, or even in endoscope per se as will be described in greater detail below) as to where to position the tool and/or as to how to orient the tool to achieve a utilitarian trajectory from outside the human and/or at least from outside the middle ear to the inner ear.

Accordingly, in an exemplary embodiment, there is a system, comprising a processor and a sensor. The processor can be microprocessor or any other logic circuit that can enable the teachings detailed herein. The processor can be part of the laptop 899 of FIG. 11 for example. Processor can also be an integrated component of the ear system endoscope 810.

The sensor can be a light capture apparatus such as light capture apparatus 822 detailed above. The sensor can also be an infrared sensor and/or can be based on sonar or radar.

In an exemplary embodiment, the sensor can be an optical sensor.

The system can also include an output device, such as the above noted display. The output device can instead or also be an audio indicator or can be a tactile indicator for that matter.

It is briefly noted that while the embodiments disclosed herein are typically in reference to the ear system endoscope system noted above, the system under discussion can also be a different kind of system, such as a system that could utilize a surgical microscope, as opposed to an endoscope. Accordingly, the various components of the systems detailed herein have an applicability to devices beyond an ear system endoscope in particular or even in endoscope in general. For example, the aforementioned displays can be displays on surgical microscopes. In this regard, the teachings detailed herein contemplate the utilization of the surgical microscope to implement the system detailed herein. Indeed, the system need not include a device configured to incise the tympanic membrane (or other tissue applicable for use with the system), at least not as an integrated component, although certainly embodiments can include such.

In this regard, with respect to the arrangements where the sensor is an optical sensor, the optical sensor can include any and all standard endoscopes in general, and such as those that utilize optical fiber bundles in particular. The optical sensors can be based on CMOS chip technology and/or CCD based technology etc., and can of course also be combined in or otherwise utilize components of surgical microscopes.

In an exemplary embodiment, the system is configured to automatically process data from the sensor indicative of sensed tissue of an ear system of a human sensed by the sensor and, using the processor, automatically identify a location in the ear system for an incision to access an area of the ear system behind the location. In an exemplary embodiment, the area that is accessed is behind the tympanic membrane. In an exemplary embodiment, the system takes advantage of the limited transparency or translucency of the tympanic membrane to identify a utilitarian location for a user to push the termination through the tympanic membrane to pierce the tympanic membrane (or for a healthcare professional to incise the tympanic membrane to establish a flat in embodiments that utilize such for example).

Briefly, in an exemplary embodiment of the system, the sensed tissue of the ear system sensed by the sensor of the system four is at least part of an ossicles of a human and system is configured to sense the location of the at least part of the ossicles through an intact tympanic membrane. By way of example only and not by way of limitation, in an exemplary embodiment, the light capture apparatus 822 can be utilized to “view” the tympanic membrane from the ear canal side of the ear system. In an exemplary embodiment, a light source can be added to illuminate the tympanic membrane and the area they are around, which light will diffuse or otherwise travel through the membrane to illuminate at least a portion of the ossicles on the other side, and/or some other tissue that can be utilized for reference. In an exemplary embodiment, the ear system endoscope 810 and variations thereof can include a light source that can be utilized to illuminate the tympanic membrane. In an exemplary embodiment, the light source is an LED while in other embodiments, the light source can be the output of a fiber optic cable, where the ultimate generation of the light is located outside the ear system while the portions of the ear system endoscope are located in the ear canal and/or in the middle ear.

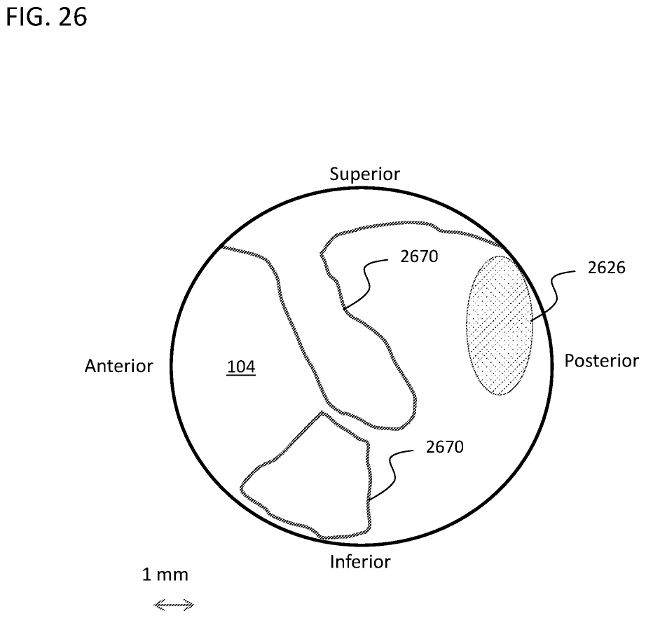

FIG. 26 presents exemplary results of the sensors with respect to tissue 2670 that can be seen or otherwise sensed through the tympanic membrane. That is, FIG. 26 presents a view looking down the ear canal toward the tympanic membrane and towards the middle ear. Shown in FIG. 26 with respect to the tissue 2670 is an elbow of the middle ear bone which is visible behind the tympanic membrane. Also seen in FIG. 26 are indicia of the spatial orientation of the tissue. Superimposed onto the image of FIG. 26 is a shaded area 2626. This shaded area can be an area where it is utilitarian to puncture through the tympanic membrane. This can be an area where, if the termination of the ear system endoscope is aligned with the wall of the ear canal, and the termination is parsed through the tympanic membrane so as to pierce the tympanic membrane within the shaded area, the termination will extend through the middle ear ultimately to a location in or proximate the round window niche.

The image of FIG. 26 can be displayed on the display detailed above. Moreover, a projected/forecasted location of piercing of the tympanic membrane utilizing the termination of the ear system endoscope can be displayed on the display detailed above. This projected/forecasted location can be moved with relative movement of the ear system endoscope to show the surgeon or other healthcare professional or user where the termination will pierce the tympanic membrane and the current alignment is maintained. This can enable the user to adjust the alignment of the ear system endoscope, which adjustment will result in the projected/forecasted location changing, where the surgeon or user adjusts the alignment so that the projected forecasted location is within the shaded area 2626. In some embodiments, the sensor can also be a range sensor that can determine or otherwise estimate the range from the end of the termination or other utilitarian point on the ear system endoscope and the tympanic membrane or other tissue of the issue. This can be utilized with the control system or feedback systems detailed herein to improve the accuracy of the forecasted/projected location. In this regard, the aforementioned processor can be configured to process the input from the various sensors to provide the output to the user indicative of the projected/forecasted location of piercing.

In an exemplary embodiment, the processor is processing a live view and/or a live data set in real time. In an exemplary embodiment, the processing is executed within five, four, three, two, or one seconds of the data capture by the sensors.

Laser range finders or the like can be utilized, or sonar radar can be utilized, or inertial navigation system components can be utilized (e.g., a gyroscope or plurality thereof can be located in the ear system endoscope, which gyroscopes can be miniature gyroscopes, and/or an accelerometer or plurality of accelerometers can be located in or on the ear system endoscope). Any device system and/or method that can be utilized to obtain data indicative of a relative positioning of the ear system endoscope or other tool that is being utilized to access the inner ear and/or of relative position of tissue of interest with the embodiments detailed herein can utilize at least some exemplary embodiments.

Still, in a utilitarian embodiment, the system is configured such that upon the identification of the location of the tissue structures 2670, or otherwise upon the sensation of those tissue structures, information is provided to the user as to a utilitarian location to incise the tympanic membrane. This information can be presented on the display noted above, or can be in the form of a tone, such as the further away or the “worse” the orientation of the tool is with respect to ultimately incising the tympanic membrane at a given location, the slower the tone is or the lower the frequency of the tone is, and the better that the orientation of the tool is the more frequent is the tone or the higher frequency of the tone. A light indicator can also be provided. In an exemplary embodiment, arrows or some vector or moment indicia can be provided to the user to provide instructions as to how to move or position the tool that will be used for incising (or for providing the therapeutic substance—any disclosure herein of orienting or positioning a tool for incising corresponds to a disclosure of an alternate embodiment of orienting or positioning a tool for delivering therapeutic substances and vice versa—indeed, as shown above, in some embodiments, the same tool is used for both).

In an exemplary embodiment, various color shades can be provided on a screen or a display or on a view to indicate good or bad areas where the tympanic membrane should be incised. In an exemplary embodiment, a gradient of colors can be used, such as where red would be a less desirable location and green or blue would be a more desirable location. Alternatively, red could be an area where the tissue should not be incised. In this regard, for example, with respect to the view of FIG. 26, the tissue 2670 can be colored in red, and the area 2626 could be colored in blue or green, and the area in between 2626 and the tissue 2670 can be colored in yellow.

In an exemplary embodiment, an inertial system can be used to provide an imbalance, however slightly, to the tool, where a user will be inclined to correct the imbalance, and thus position the tool in a utilitarian manner. By way of example only and not by way of limitation, gyroscopes could be forcibly positioned, where the counterforce resulting from the forcible position imparts a sensation of a tactile imbalance or some other imbalance.

Indeed, in an exemplary embodiment, the system can be configured to operate in a manner analogous to a homing missile or the like. In this regard, embodiments can utilize the technique of “painting” a target with an infrared or a laser dot. The system can be configured to “home” in on the target, at least with respect to providing feedback/indicia to the user with respect to how to position and move the tool. Some embodiments can utilize the image recognition features of a cruise missile or the like. In this regard, cruise missiles are often configured to recognize terrain images and determine a position based on an automatic electronic analysis of the image. These techniques can be utilized, albeit modified, with respect to the systems detailed herein.

Any device, system, and/or method that can enable feedback indicative of the ultimate location that the tool will incise the tympanic membrane to be provided to the user can be utilized in at least some exemplary embodiments.

Consistent with the teachings detailed above, the output device of the system can be a visible indication. In an exemplary embodiment, this can be a visible indication projected on the location in the ear system, such as on the tympanic membrane. In an exemplary embodiment this can be a laser dot or the like that is beamed from a laser emitter of the ear system endoscope or other device that includes the sensor of the system. For example, a green dot or a blue dot can be projected on to the location of the tympanic membrane that corresponds to the shaded area 2626. The system can be configured to identify or otherwise utilize data indicative of the location of tissue structures 26702 identify the area 2626 that has utilitarian value with respect to incising through the tympanic membrane. The system can then project the laser dot or even a laser illuminated area, or, by adjusting the beam of the laser quickly enough, the outer periphery of the area can be superimposed onto the tympanic membrane.

In another exemplary embodiment, the system can be configured to more permanently mark the location on the tympanic membrane. This can be done by providing a superficial burn mark on the outer surface of the tympanic membrane. In an exemplary embodiment, this can be done by providing an ink stain or an ink mark on the tympanic membrane. Any device, system, and/or method that can enable the establishment of a visual indication to be projected on the location of the tympanic membrane or other tissue for that matter can be utilized in at least some exemplary embodiments.

It is briefly noted that in an exemplary embodiment, the marking can be confirmed by a healthcare professional, and then a robotic insertion can be executed utilizing an endoscope coupled to or otherwise part of a robot system, as will be described by way of example below. Briefly, it is noted that the surgeon himself or herself could mark the locations, and the robotic system could automatically execute some or more or all of the method actions disclosed herein.

In an exemplary embodiment, the sensor is an optical sensor, and the output device is a display. Further, the system is configured to display an image of the location obtained with the optical sensor on the display, and overlay the identified location on the image. As noted above, the image of the location can be that of the tympanic membrane and/or the components in back of the tympanic membrane. Still further, embodiments can include utilization of the techniques herein where the image of the location is the round window. With respect to the round window, the area about the round window can be utilized for reference. Indeed, owing to the fact that the round window niche establishes an overhang over the round window, the overhang and the approach thereto can be utilized as reference locations (as opposed to the ear canal, which is generally a “straight shot” from the outside to the tympanic membrane. Indeed, in some embodiments, prior movements and/or orientations of the tool can be taken into account to determine the current location of the tool or otherwise estimate the specific and tissue proximate to the tool.

Embodiments so far have been described in terms of a display or an image presentation device that is hard mounted to the tool or relatively remote from the tool, such as is the case with respect to the utilization of the laptop computer noted above. Some embodiments include a middle ground, where the display is not hard mounted or otherwise directly supported by the remainder of the tool that is being used, what is in close proximity to the head of the recipient. By way of example only and not by way of limitation, in an exemplary embodiment of the system under discussion, the display is located within 100, 90, 80, 70, 60, 50, 40 30, 29, 28, 27, 26, 25, 24, 23, 22, 21, 20, 19, 18, 17, 16, 15, 14, 13, 12, 11, 10, 9, 8, 7, or 6 inches, or any value or range of values therebetween in 0.1 inch increments of the sensor of the system. In the case of multiple sensors, these distances are in relation to the sensor that is the furthest inside the person or will be the furthest inside the person when used as intended. This can be utilitarian in that a surgeon or other user meeting one or more of the human factors engineering criteria detailed herein, for example, can be able to stand up straight and look at a screen on the other side of the patient without having to bend their neck, in contrast to that which might be the case when looking down a surgical microscope).

FIG. 27 depicts an exemplary system according to an exemplary embodiment, including the ear system endoscope 810 (but which is a proxy for any of the tools detailed herein), which, in an exemplary embodiment, and a portable handheld device 240 having a wireless link 230 with the endoscope 810 (although in some embodiments, a wired link can be utilized). In an exemplary embodiment, the system is configured such that endoscope 810 and the portable handheld device 240 (e.g., a portable cellular telephone, such as by way of example only and not by way of limitation, a smart phone, as that phrase is utilized generically) have a relationship. By way of example only and not by way of limitation, in an exemplary embodiment, the relationship is the ability of the smartphone to provide indicia to the user along the lines above (e.g., visual display) based on data obtained via the wireless link 230 and/or to utilize the onboard processing features of the smart phone as the processor of the system. (Note that in embodiments where the laptop is utilized, the processor of the laptop can be utilized to the processor of the system.)

In an exemplary embodiment, the handheld device 240 can be placed next to the head of the patient. In an exemplary embodiment, the handheld device 240 can be placed on top of the head next to the pinna, and the image displayed on the screen of the smart phone 240 can be that of the inside of the ear canal and/or the inside of the middle ear, etc. In an exemplary embodiment, where for example, the patient is facing directly upwards towards the ceiling, the handheld device 240 can be placed to the left or the right of the pinna (depending on, for example, the handedness of the surgeon) so that the surgeon can view the display as the surgeon is inserting the tool in the ear canal and/or through the middle ear. That said, in an exemplary embodiment, the audio system of the handheld device 240 can be used to provide audio instructions to the surgeon and/or to provide audio data to the surgeon regarding the positioning of the tool and/or the tissue relative to the tool.

In an exemplary embodiment, onboard orientation related components of the handheld device 240 can be utilized to orient the handheld device relative to, for example, the direction of gravity (these features are common to smart phones—the auto rotate feature for example). In an exemplary embodiment, an application can be utilized to instruct the surgeon on how to position the smart phone 240, or otherwise to obtain data relating to the current orientation of the smart phone as it relates to the orientation of the tool that is being utilized. The system can synchronize relative orientation of the two so as to provide utilitarian data to the surgeon with respect to instructions to the surgeon or data to the surgeon.

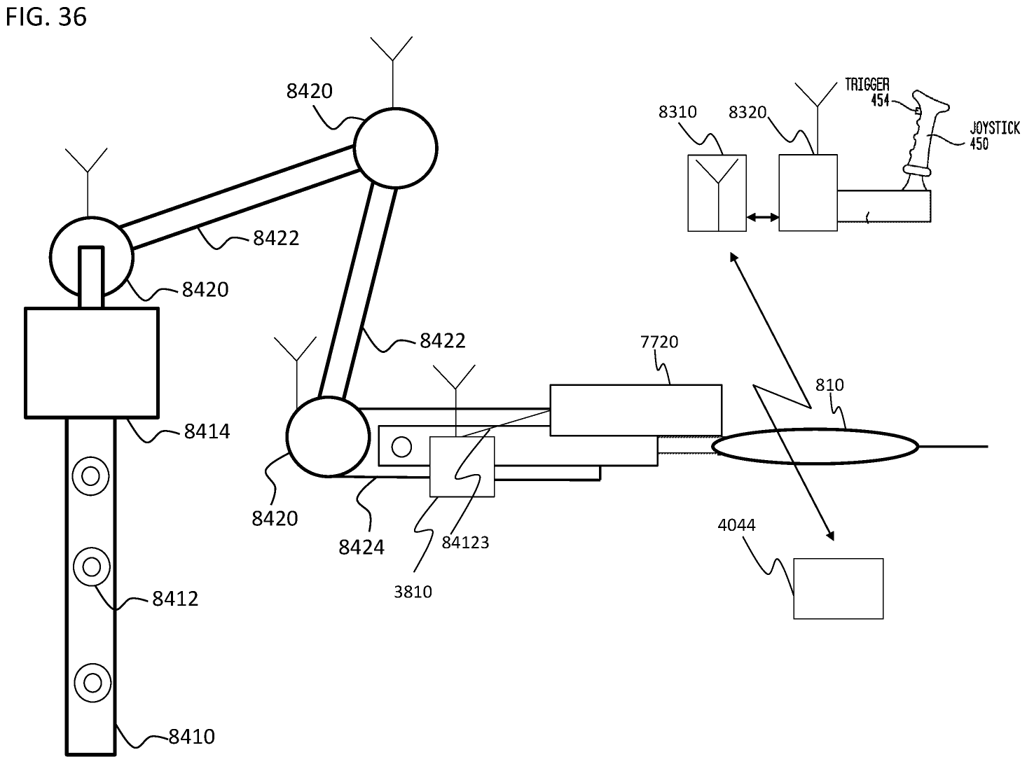

Conversely with respect to the scenario of use where the displayed all the components are in the same room as the patient, in an alternative embodiment, the data obtained by the sensors can be streamed or otherwise provided in a real-time manner over the Internet or over a telephone system or over a communication system to a remote location more than miles away (e.g., more than 10, 100, or 500 miles or more away) to a healthcare professional. As will be shown below, in an exemplary embodiment, a robotic system can be utilized with the endoscope. Briefly, with respect to the embodiment of FIG. 36, the data from the endoscope can be wirelessly or wired transmitted to the remote location. Embodiments thus include executing a noninvasive procedure remotely.

In an exemplary embodiment, the system is configured to automatically process data from the sensor indicative of second sensed tissue of the ear system of the human sensed by the sensor (for example, the initial sensed tissue could be the tympanic membrane, and now the second sensed tissue is the round window or the tissues thereabout, etc.) and, using the processor, automatically identify a second location in the ear system for an incision to access a second area of the ear system behind the second location, wherein the second area is inside a cochleovestibular system of ear system. In an exemplary embodiment, the second area is inside the cochlea of the ear system.

Briefly, returning to FIG. 27, FIG. 27 presents an exemplary embodiment where a processor 2727 is hard mounted to the ear system endoscope 810. In this embodiment, the processor 2727 receives a signal from the light channel/the optical channel 820 (or a signal channel/sensor channel, in embodiments where for example the sensor is not a light capture device). The data received over channel 820 can be processed by the processor so that the processor can execute one or more or all of the functions detailed herein and/or variations thereof. In this regard, the processor 2727 can be a data evaluation suite (which can include a housing that houses the processor, a “jack” to receive the light from the light channel 820, a sub processing system that can convert the light provided by the light channel 820 to electronic data/electrical signals, and an electrical conductivity suite that conveys the electrical data to the processor contained in the housing, etc.) that can be configured to receive data indicative of light and convert that to a medium in which the processor can evaluate the data. In an exemplary embodiment, light channel 820 converts the light captured by the endoscope to an electronic signal that can be provided to the processor and evaluated by the processor. The processor 2727 in general, or the data evaluation suite in particular, can be configured to interact with an off-the-shelf endoscope by placing the data evaluation suite onto the light channel 820 or otherwise the output of the light channel 820.

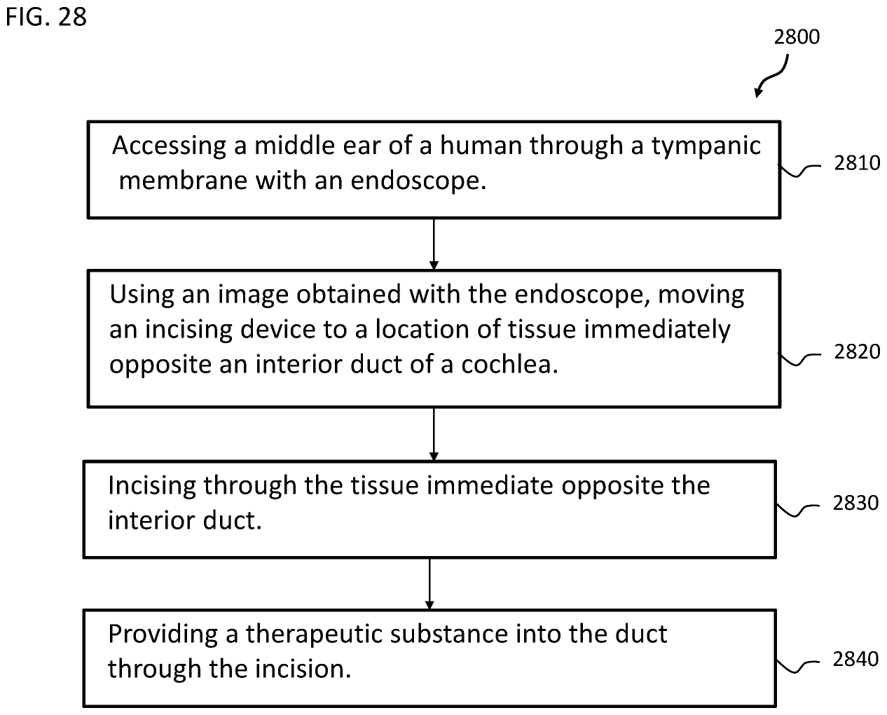

Embodiments include methods. FIG. 28 presents an exemplary flowchart for an exemplary method, method 2800, which includes method action 2810, which includes the action of accessing a middle ear of a human through a tympanic membrane with an endoscope. This could be the endoscope 820 noted above, or another variation thereof, or a traditional endoscope for that matter. Method 2800 further includes method action 2820, which includes the action of using an image obtained with the endoscope, moving an incising device to a location of tissue immediately opposite an interior duct of a cochlea. In an exemplary embodiment, the action of moving the incising device can be executed utilizing any of the guidance techniques detailed herein and/or variations thereof. In an exemplary embodiment, method action 2820 is executed by a surgeon holding in incising device, and the feedback systems detailed herein providing indicia to the surgeon as to where a reference component of the tool (e.g., tip of a termination) is located relative to tissue of interest and/or how to position the tool so as to execute a utilitarian incision.

Method 2800 further includes method action 2830, which includes incising through the tissue immediate opposite the interior duct. In an exemplary embodiment, this is executed utilizing the drill bits detailed herein. In an exemplary embodiment, this is executed utilizing the piercing tools detailed herein. Any device, system, and/or method of incising through the tissue whether that tissue is bone or membrane or cartilage can be utilized in at least some exemplary embodiments.

Method 2800 further includes method action 2840, which includes the action of providing a therapeutic substance into the duct through the incision. The action of providing the therapeutic substance can be executed via any of the associated teachings detailed herein. In an exemplary embodiment, this can include utilizing the combined ear system endoscope noted above. In an exemplary embodiment, this can include utilizing a tool that is completely separate from the tool that is utilized to execute the incising. Still, consistent with embodiments detailed herein, method action 2840 can be executed utilizing the tool that was used to execute the incising, whether the action is executed by passing the therapeutic substance through the exact tool that was used, or by using another feature of the tool. In this regard, in an exemplary embodiment, the device that is utilized to provide therapeutic substance and/or the action associated there with results in the therapeutic substance passing within at least 3, 2.5, 2, 1.5, 1, 0.75, 0.5, 0.25 inches, or any value or range of values therebetween in 0.05 inch increments of the working component that was used to execute the action of incising (e.g., tip of a drill bit, tip of the termination, etc.). In an exemplary embodiment, the action of providing the therapeutic substance into the duct is executed by passing the therapeutic substance within half an inch from a working surface of a tool used to incise through the tissue while the working surface is in a head of a human in which the tympanic membrane is located. In an exemplary embodiment of this method action can be executed utilizing the drill of FIG. 24, where the orifice 2450 extends through the working surface that is used to incise through the tissue.

But in any event, in an exemplary embodiment, the endoscope utilized in method action 2810 is part of an integral apparatus that is configured to incise through the tissue, which integral apparatus to be the ear system endoscope 810 above. In an exemplary embodiment, the endoscope is part of an integral apparatus that is configured to incise through the tissue.

Consistent with the embodiments noted above, in an exemplary embodiment, electronic logic is utilized to identify the location of incising. This can be done automatically. In an exemplary embodiment, digital processing is utilized to identify the location of incising.

In an exemplary embodiment, the action of providing the therapeutic substance into the duct is executed by passing the therapeutic substance through a tool used to execute the action of incising. This will be executed by way of example by utilizing the ear system endoscope 810. This as contrasted to an embodiment where, for example, a separate drill tool is utilized to execute the action of incising, and that drill tool is removed and a conduit of a drug delivery tool is inserted into the drilled hole to deliver the therapeutic substance.

Consistent with the teachings detailed above, the tissue that is incised through in method action 2830 is one of a promontory of a cochlea or a round window of a cochlea.

In an exemplary embodiment of the method 2800, during the action of providing therapeutic substance, there is no permanent component at the location of incising. In an exemplary embodiment, the method further includes moving a human in which the duct is located from a room where the action of incising was executed, and during the action of moving, there is no permanent component at the location of incising. The action of moving could be executed by moving a gurney or an operating table upon which the person is lying, or can be executed by the person himself or herself by walking out of the room such as where this method is executed in and outpatient basis.

During the action of incising and during the action of providing, in an exemplary embodiment, a human in which the duct is located is under only local anesthesia. In an exemplary embodiment, during the action of providing, the only incision(s) into a human head in which the duct is located is through an ear system of the human. This as opposed to, for example, that would be the case if the middle ear reached by incising into the area behind the pinna.

In view of the above, it can be seen that in an exemplary embodiment, there is a device, comprising a therapeutics substance delivery apparatus and an incisor, wherein the device is a minimally invasive inner ear therapeutic substance delivery device. This device can correspond to the ear system endoscope noted above. In an exemplary embodiment, the device is configured to reach the inner ear through a passage through the tympanic membrane, which passage is no greater in diameter than 10, 9, 8, 7, 6, 5, 4, 3, 2, or 1 mm, or any value or range of values therebetween in 0.05 mm increments to incise through tissue and deliver a therapeutic substance. In an exemplary embodiment, the incisor is at least one of a drill bit configured to drill through a promontory of a cochlea of a human, or a conduit configured to pierce a round window of the human with a fully intact round window niche. In an exemplary embodiment, the termination can be a 2, 3, 4, 5 or 6 mm in diameter termination, and can include one or two or three or more working channels, and thus there can be utilitarian value to be able to extend through an opening of a pertinent size, and this can be for both piercing the tympanic membrane (myringotomy) but also for introducing the tool to the middle ear using a so called tympanomeatal flap, where the entire tympanic membrane is lifted towards the ceiling of the ear canal for maximum access to the middle ear.

In an exemplary embodiment, the therapeutic substance delivery apparatus is configured to deliver a solid therapeutic substance to the cochlea. This can be accomplished for example via a direct straight line therapeutic substance delivery path which can be achieved via drilling through the promontory. This as contrasted to, for example, the embodiments that extend from the tympanic membrane to the round window niche and curve there around to reach the round window. Accordingly, some embodiments include delivering therapeutic substances along a path that extends from a tympanic membrane to a duct of a cochlea that deviates no more than 5, 4, 3, 2, or 1 mm from a straight line extending from the tympanic membrane to the duct of the cochlea (the path could widen or narrow—if a straight line is superimposed upon the path, there will be a therapeutic substance delivery channel, or more accurately, a portion of that channel within the aforementioned values of that line—if the entire channel envelops the line, and that would meet the feature because the channel would be within 5 mm for example (it would be within zero millimeters, of course)).

Conversely, some embodiments include delivering therapeutic substances along a path that extends from the tympanic membrane to a duct of a cochlea that includes a curve or a dogleg or the like so that the path deviates by at least 30, 35, 40, 45, 50, 55, 60, 65, 70, 75, 80, 85, 90, 95, 100, 105, 110, 120, 125, 130, 135, 140, 145, or 150 degrees, or any value or range of values therebetween in 1 degree increments. In an exemplary embodiment, this curve or dogleg takes place at the round window niche/around an overhang of the round window niche. In an exemplary embodiment, this exists within 1, 2, 3, 4, 5, 6, 7, 8, 9, or 10 mm, or any value or range of values therebetween in 0.1 mm increments from an end of the drug delivery device. In an exemplary embodiment, this curve or dogleg exists in the device from before the time that the device passes the tympanic membrane or even enters the head of the human to the time that the therapeutic substance has commenced delivery. In an exemplary embodiment, this curve or dogleg exists only after the portion associated therewith has passed through the tympanic membrane and/or has reached the round window niche or an area proximate the round window niche.

In an exemplary embodiment, concomitant with the teachings detailed above, the therapeutic substance delivery apparatus is configured to deliver a fluid therapeutic substance to the cochlea. In some embodiments, the incisor is the drill bit and in some embodiments, the drill bit is a spherical drill bit, and in other embodiments, the incisor is a chamfered portion of the conduit, and the device is configured to deliver the therapeutic substance through the conduit. Consistent with the teachings detailed above, the device can include an optical scope configured to at least view a distal end of the device.

It is noted that in at least some exemplary embodiments associated with drilling through the promontory of the cochlea, the method of accessing the cochlea in general, and the duct(s) thereof in particular, is executed without drilling completely through to the ducts. In an exemplary embodiment, a drill, which can be a conventional drill bit, in which can be a spherical drill bit without the drug delivery features associated there with, is utilized to drill down into the promontory to a location proximate the duct(s) or just to the location where the inner tissue of the cochlea can be seen. The surgeon or other healthcare professional removes the drill bit from the excavation, and then utilizes a tool to establish the opening in the final portion of the tissue to reach the duct, and to open a hole that is sufficiently large enough to access the duct of the cochlea. In some embodiments this might be a chipping tool, or a tool that can be used by applying pressure over a concentrated area, to “break” through to the final area, or to scratch through. In this embodiment, a drill bit is not utilized to make the final breakthrough, or, the drill bit might be utilized, but there is no or minimal rotation associated with the working end of the tool. In an exemplary embodiment, the working end of the tool is limited to a total rotation of 270, 250, 220, 180, 150, 130, 100, 90, 80, 70, 60, 50, 40, 30, 20, 10, 9, 8, 7, 6, or 5 degrees or less or any value or range of values therebetween in 1° increments during the entire procedure of breakthrough. By way of example only and not by way of limitation, if a drill bit is used, the drill bit is not completely rotated 360°. It could be that the drill bit is rotated 77° back and forth for example or just 77° in one direction. The point is that the final breakthrough in at least some exemplary embodiments is established utilizing a nonrotating tool. In an exemplary embodiment, the final breakthrough in at least some exemplary embodiments is established utilizing a tool that might rotate during use, and potentially on purpose, but that rotation is entirely a result of hand rotation/human induced rotation (as opposed to powered rotation).

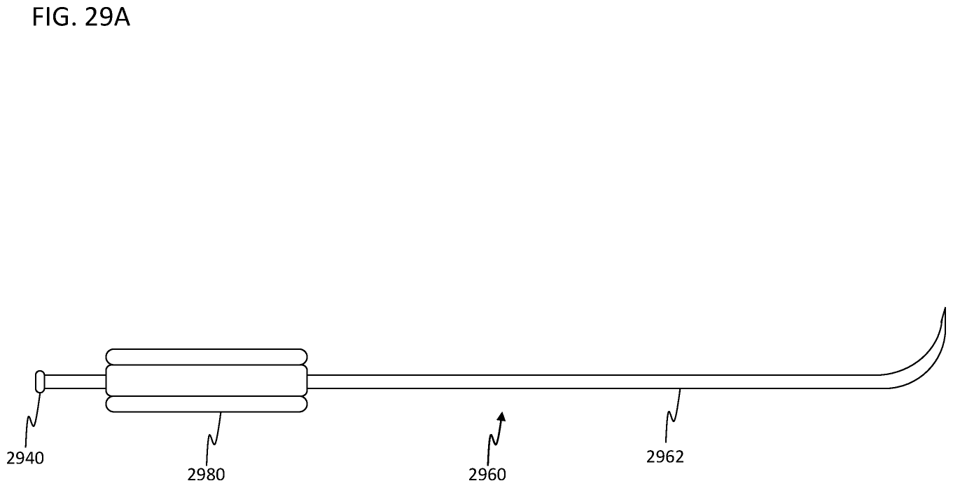

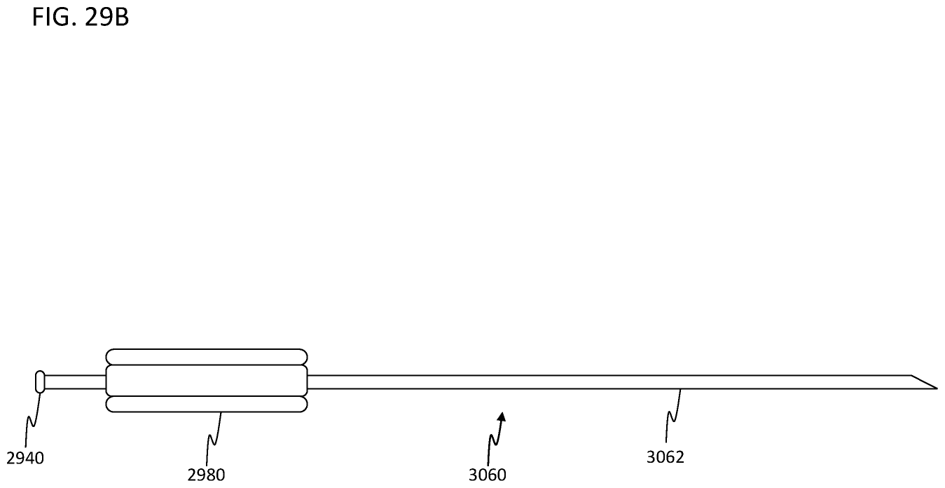

FIGS. 29A and 29B present exemplary embodiments of such a chipping tool that can be utilized to complete that last bit of tissue removal so as to access the duct. These figures present tools 2960 and 3060 respectively that includes handles 2980 through which extends a conduit 2962 and 3062, respectively. At the end of both conduits is a port 2940. In this exemplary embodiment, the port can be attached to a therapeutic substance delivery device or supply thereof, such as a reservoir, so that therapeutic substance can be provided to the tool, and thus provided out the end of the conduit.

In both of the embodiments of the tools of FIGS. 29A and 29B, the end of the conduit is chamfered so as to establish a sharp or relatively sharp end, or an and that is sufficiently narrow so as to fit into the excavation and to be used to complete the chipping through to the duct. The embodiment of FIG. 29A includes a curved end, whereas the embodiment of FIG. 29B includes a straight end.

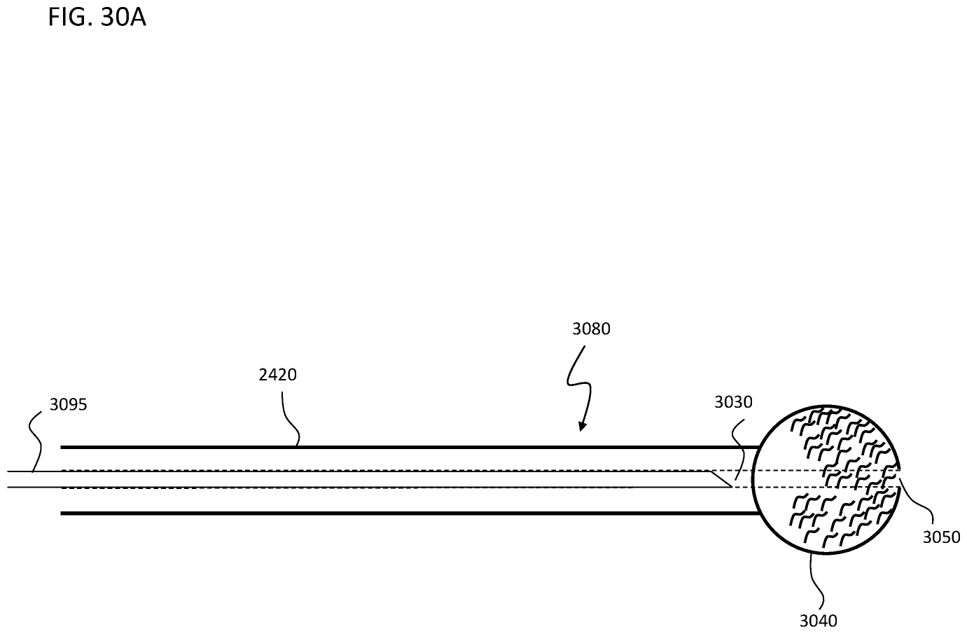

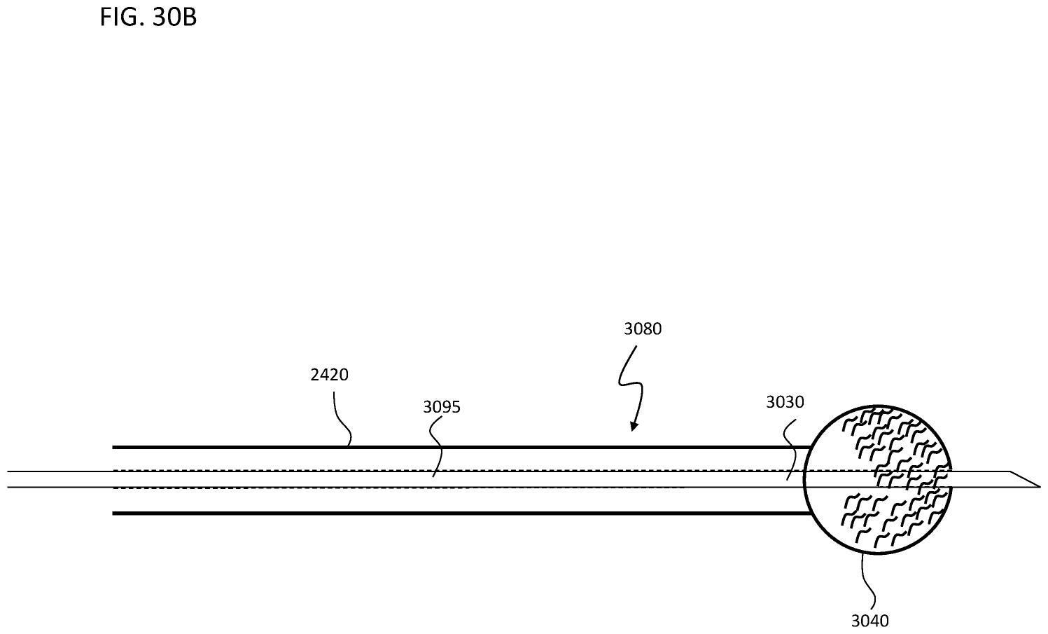

The embodiments of FIGS. 29 and 30 can be utilized such that after the tools thereof are used to break through the final portion of the tissue to reach the duct of the cochlea, therapeutic substance is delivered utilizing those tools through the conduits thereof.

Accordingly, in an exemplary embodiment, there is a method that includes drilling down through the promontory a distance of at least 70, 75, 80, 81, 82, 83, 84, 85, 86, 87, 88, 89, 90, 91, 92, 93, 94, 95, 96, 97, 98, or 99%, or any value or range of values therebetween in 0.1% increments of the total distance from the outer surface of the promontory to the inside of the cochlea that would be required to reach the duct in a manner that would enable therapeutic substance delivery to the duct. Then, a chipping tool is utilized to chip away at the remaining tissue so as to completely access the duct. Then, utilizing the chipping tool, therapeutic substance is delivered through the now complete opening to the duct.

The concepts associated with the chipping tool can be incorporated into a drilling tool and/or into the endoscope. Indeed, in an exemplary embodiment, the termination 860 can be utilized to execute the method of chipping through the final portion of tissue that remains after drilling is completed. That said, FIG. 30A depicts an exemplary embodiment where a drill bit is combined with a chipping tool. Particularly, a drill bit and conduit assembly is shown in FIG. 30A. FIG. 30A depicts the assembly 3080 that includes a circle drill bit 3040 mounted on a shank 2420 through which extends a lumen 3030 which extends to an orifice 3050 at the end of the spherical drill bit 3040. Also shown is a conduit 3095, that can be that of a termination of a syringe or any of the other conduits detailed herein, which conduit 3095 has a distal end that a sharp or otherwise is configured to chip through the tissue in a utilitarian manner. In an exemplary scenario of use, the drill bit and conduit assembly 3080 utilized with the conduit 3095 in a retracted state as shown to drill through or otherwise excavate the tissue of the promontory. Then, upon the completion of drilling, the chipping tool/conduit 3095 is extended as shown in FIG. 30B. Then, the conduit 3095 is utilized to chip away at the remaining tissue to access the duct of the cochlea. The conduit 3095 is then inserted through the opening into the duct, and therapeutic substance is directed through the lumen of the conduit into the duct.

FIG. 24 presents an exemplary drill bit 2410 that can have utilitarian value with respect to the teachings detailed herein. This drill bit can be located at the end of the ear system endoscope and/or can be utilized separately from the ear system endoscope. Drill bit 2410 includes a shank 240 through which a lumen 2430 extends to and orifice 2450 at the surface of the working end 2440 of the drill bit. Drill bit 2410 is a spherical drill bit, which includes a roughened surface 2450, which roughened surface is utilized to remove bone at the promontory for example so as to form a cochleostomy. FIG. 25 depicts utilization of the drill bit 2410 to form an excavation 2525 at the promontory of the cochlea, which excavation extends into the duct 181 of the cochlea as seen. In this exemplary embodiment, the therapeutic substance can be delivered through the conduit 2430 upon reaching the duct 181 of the cochlea. By way of example only and not by way of limitation, the orifice 2450 is located at a position that will not interfere with the overall drilling the features of the drill bit 2410, and then, upon the completion of drilling, the drill bit can be stopped, and the therapeutic substance can be channeled into the cochlea as shown, providing that the orifice 2450 is positioned properly. Such verification can be established utilizing the optics of an endoscope, such as the optics of an endoscope of which the drill bit 2410 is a part, or by eyeballing the arrangement if the more invasive surgical procedure is utilized to reach the promontory. It is noted that in some embodiments, the drill bit 2410 sized and dimensioned so as to fit through a puncture through the tympanic membrane, while in other embodiments, a tympanic membrane flap is established so as to reach the promontory with the end of the drill bit 2410.

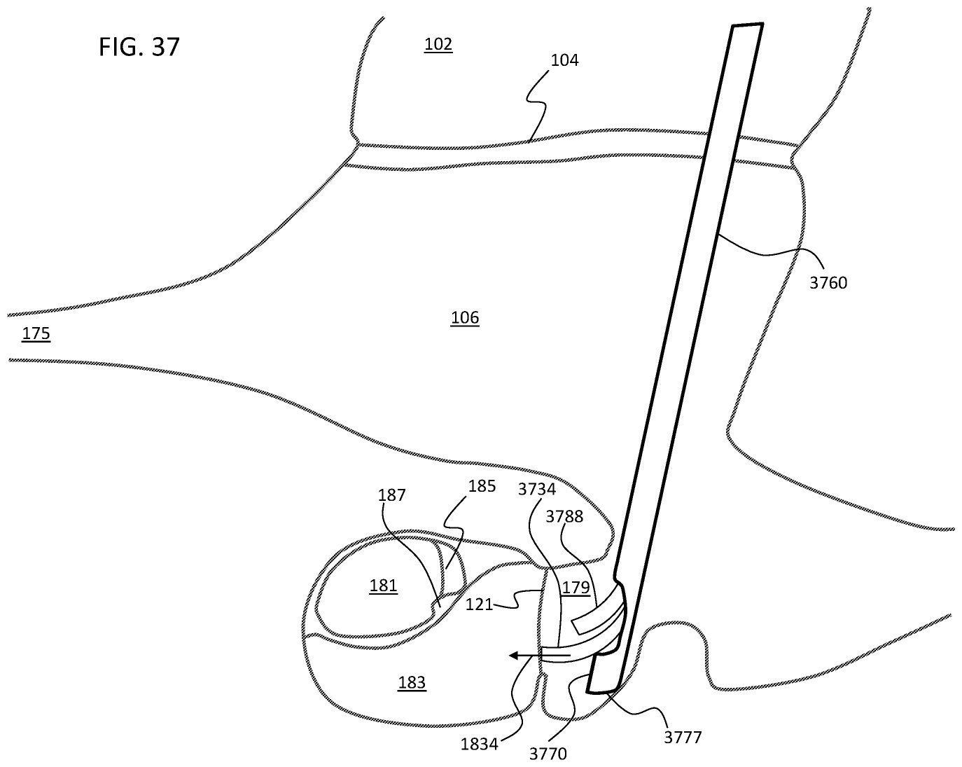

FIG. 37 depicts another exemplary embodiment where the termination is configured so that instead of the conduit and other components exiting out of the very end, the components exit out a side of the termination. As an initial matter, the embodiment of FIG. 37 has a distal end that is curved as opposed to pointed. In this regard, the distal end 3777 of the termination can be one that is a “do no harm” component. As seen, the end of the termination 3777 can be rounded or otherwise can be blunt, as opposed to the sharp piercing features associated with the embodiments above. Also, as seen, the end of the termination 3777 can be a closed end.

In this regard, in at least some exemplary embodiments, the action of piercing or otherwise puncturing or extending through the tympanic membrane can be executed utilizing a tool that is separate or otherwise not part of the ear system endoscope. Indeed, such can be the case with respect to the embodiments where a flap is opened into the tympanic membrane. Accordingly, in at least some exemplary embodiments, the end of the termination is not sharp. Here, it can be blunt, which can be a flat face or a rounded hemispherical shape or a partial spherical shape, etc.

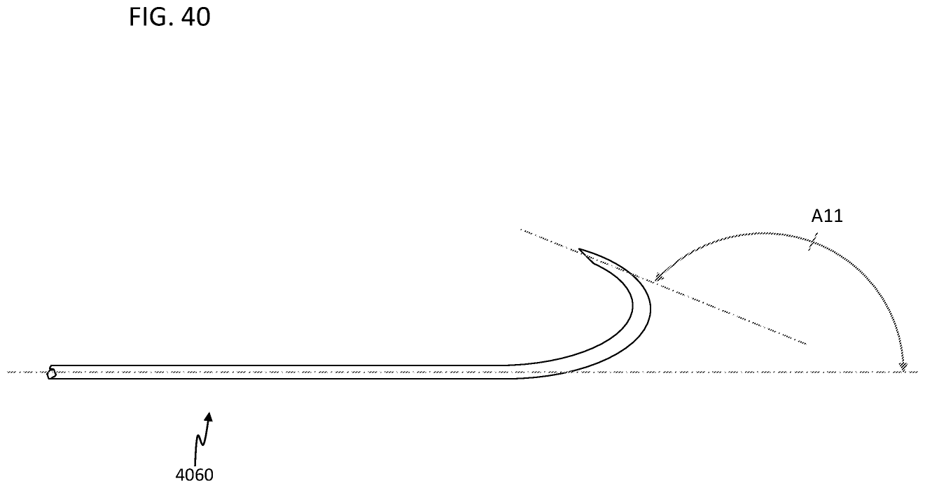

Also as can be seen, the conduit 3737 which here is a blunt conduit, but which could be a sharp ended conduit, is shown exiting from the cylindrical wall of the termination 3777, as opposed to out of the end of the termination 3777. In an exemplary embodiment, the configuration of the termination in general, and the interior thereof in particular, can be such that the termination provides a ramp like feature or otherwise a guide to guide or force the conduit away from the termination or otherwise away from the longitudinal axis of the termination in general, and the longitudinal axis of the termination associated with that where the termination extends the tympanic membrane in particular, as the conduit is further extended into the ear system/through/out the termination. In an exemplary embodiment, the ear system endoscope is configured so as to change a direction of trajectory of the conduit (or the termination for that matter), at least and/or equal to 5, 10, 15, 20, 25, 30, 35, 40, 45, 50, 55, 60, 65, 70, 75, 80, 85, 90, 95, 100, 110, 120, 130, 140, 150, 160, 170 or 180 degrees or any value or range of values therebetween in 1° increments relative to the trajectory of the conduit at the location where the conduit is passing through the tympanic membrane and/or at the location where the conduit is completely above the promontory. (FIG. 40 depicts an exemplary termination 4060 that has a bend at the distal portions thereof. The bend is measured from the longitudinal axes of the termination at the location where it passes through the tympanic membrane (e.g., which could be represented by the portion thereof pointed to by the arrowhead of the reference 4060 for example) and the local longitudinal axis of the end of the termination. The angle is angle A11 as can be seen, which angle can be any of the just detailed angles as just noted (the angle A11 can also be zero). These values can also be applicable to the conduit and/or to the optical cable, for example. Is also briefly noted that in an exemplary embodiment, the termination can be pre-bent, and/or can be such that a surgeon or user or the like can bend it by hand without damaging the termination. This can enable customization for a given patient and/or for a given surgeon. Is also noted that such bending is not mutually exclusive with the other changes in shape detailed herein.)

In an exemplary embodiment, there is a bend and/or a plurality of bends one or more vector deviations from the longitudinal axis in the termination between the handle and the distal end of the tool. Any one or more of these bends collectively or singularly can result in the vector of the termination changing over any one or more the aforementioned angles.

It is noted that while the embodiment depicted in FIG. 37 depicts the body of the termination as the feature that changes the trajectory of the conduit, another component or an additional component can be utilized to change the trajectory. By way of example only and not by way of limitation, in an exemplary embodiment where, for example, something more than 90° turn is deemed to be utilitarian or otherwise is desired, a component on the conduit can be located at a specific location therealong, such that when this component reaches the “ramp” of the termination, this component further pushes the conduit away from the ramp, and thus adds to the overall angle. In an exemplary embodiment, a telescoping feature can be used to further push or pull a portion of the conduit to further change the trajectory of the conduit. A trapeze system that automatically or controllably extends or goes into position can be used. In some embodiments, the conduit has a memory or otherwise is made of a memory material, where the natural state of the conduit in a relaxed state or otherwise unrestrained state is to curve (or at least a portion thereof—part of the conduit can have a memory that is such that in the unrestrained state, the conduit curls, and part of the conduit can have a memory such that in the unrestrained state, it is straight (which might be considered no memory)). In some embodiments, a combination of the two features can be utilized.

Also shown in the embodiment of FIG. 37 is an optical cable 3788, which is shown also extending from the opening in the cylindrical wall of the termination 3760. In an exemplary embodiment, the conduit 3734 and the optical cable 3788 can extend down the same lumen of the termination 3760. Conversely, in an exemplary embodiment, there can be a plurality of separate lumens in the termination for the respective components. In an exemplary embodiment, the termination can include a separate conduit for the optical cable 3788 on the outside of the termination (there could be a “double barreled termination”). In an exemplary embodiment, the optical cable can be moved within the termination in the longitudinal direction in a manner concomitant with that of the conduit 3734. In an alternate embodiment, the optical cable can be fixed with respect to longitudinal movement. Indeed, in an exemplary embodiment, the conduit 3734 can also be fixed with respect to longitudinal movement. In an exemplary embodiment, predetermined distances for extension laterally away from the longitudinal axis of the termination can be relied upon. By way of example only and not by way limitation, a component, such as a wedge or a ramp that can move relative to the longitudinal axis of the termination 3760, can be pulled upward away from the distal end, and the wedge can provide a force that pushes the conduit and/or the optical cable outward away from the longitudinal axis (underneath the component—between the component and the far wall of the lumen of the termination. The positioning and/or the amount of travel of this wedge or ramp can determine the angle and/or the distance of extension of the cable and/or conduit away from the longitudinal axis. For example, the more that this wedge/ramp is pulled upward the more that the conduit extends outward (like a wood shaving for example). Also note that in an exemplary embodiment, the wedge/ramp could be moved in the opposite direction so as to provide retraction of the various components. Another component could be located on top of the conduit, etc., that is movable and can apply an opposite force to cancel the movement, or further refine the positioning, etc. Also, components can be located on the left and or the right side of the conduit, etc., so as to push and/or pull the conduit in the left or right direction, etc.

By way of example only and not by way limitation, the conduit and/or the cable, can have memory so as to resist the movement outward, so that in the relaxed state, the natural tendency is for the conduit, etc., to be within the outer walls of the termination 3760.

It is briefly noted that the end of the optical cable 3788 that faces the distal end of the conduit 3734 constitutes the direction of view of the optical cable 3788—that is, with respect to the view of FIG. 37, the light that is captured is the light that comes from the very distal end of the conduit 3734, and thus the viewer could see the distal end of the conduit 3734 in direct contact with the round window 121.

Further, in an exemplary embodiment, instead of or in addition to the various features just detailed to extend or otherwise position the conduit 3734 and/or the optical cable 3788, these components can have memory such that when these components are rotated a certain amount about the longitudinal axis thereof, the components can then spring outward away from the longitudinal axis of the termination. For example, during the insertion phase of the termination through the tympanic membrane, etc., the conduit 3734 can be at a rotational orientation such that the memory of the conduit pushes the top of the conduit towards the wall of the lumen of the termination 3760 that is located furthest away from the round window with respect to the orientation of FIG. 37. Then, by rotating the conduit 3734, say, 180° for example, the conduit 3734 can “spring” outward because it is now in the relaxed state. Thus, movement/position can be achieved of the conduit and/or the optical cable without moving those components and the longitudinal axis.

Still, with respect to an exemplary embodiment that may or may not utilize the memory features of the conduit and/or the optical cable, there could be a feature of the termination 3760 that is moved, which structural feature, such as a band or a ring or some other component, restrains the conduit and/or the optical cable when in a first position by “covering” that component, and then when that structural feature is moved, which may be the case when a pulling action on a cable or some other tensioning structure is applied through a lumen of the termination, or a pushing action can be provided, the force restraining the various components is now relieved, and the components can “spring” outward. Electrical actuators can be used in some embodiments, to release the holding component.

It is briefly noted that in an exemplary embodiment, the conduit can be made of any of the materials detailed herein that are utilized to make the termination. In an exemplary embodiment, the conduit can be nitinol, or can be a spring based metal material such as a spring based stainless steel material, etc. A polymer-based material can be utilized instead and/or an alternatively to this. Moreover, the distal end of the conduit can be made of a harder material, such as a stainless steel or the like, which can have utilitarian value with respect to piercing the round window, and the remainder of the conduit can be made of a polymer for example. In an exemplary embodiment, materials are utilized in guidewires or the like can be utilized to make the conduit and/or the terminations.

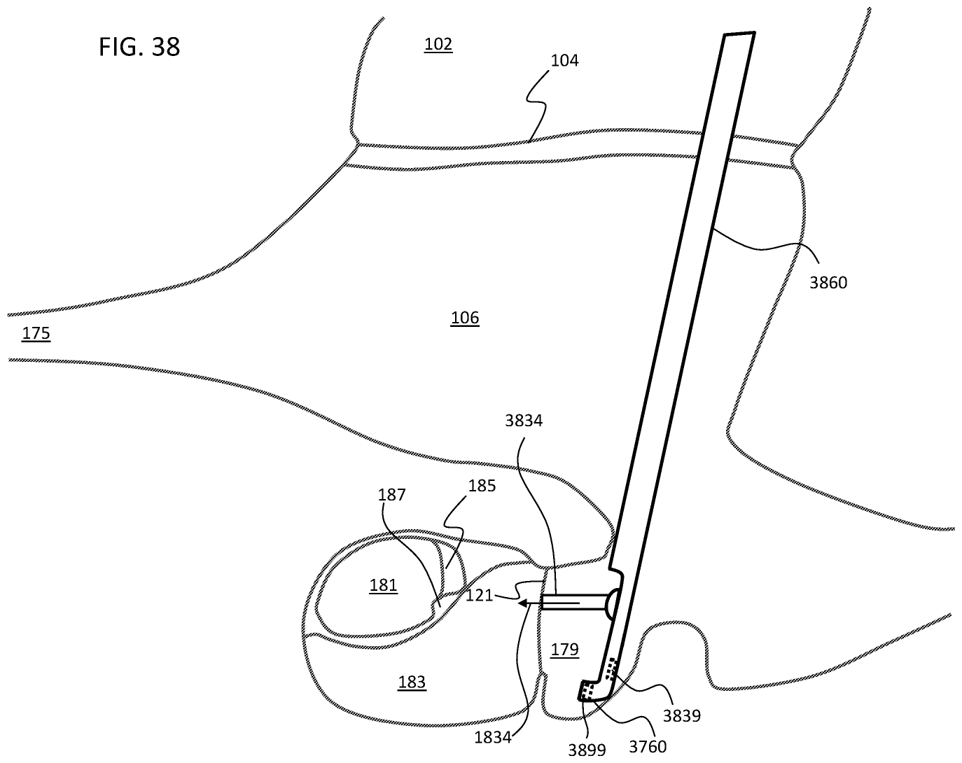

FIG. 38 presents an alternate exemplary embodiment. Here, there is a termination 3860 that has an opening at the distal portions thereof on one side, concomitant with the embodiment of FIG. 37. In this embodiment, the conduit 3834 is a turret like device that can be moved in a manner analogous to moving a cannon barrel of a tank. The conduit 3834 can be configured to articulate in one or two axes. In an exemplary embodiment, the conduit 3834 can telescope, thus enabling articulation in a third axes. In an exemplary embodiment, the conduit 3834 can “swing” in the plane of FIG. 38 and/or in a plane that is normal to the plane of FIG. 38 which plane is aligned with the horizontal. In some embodiments, the conduit 3834 can swing in the plane that is normal to the plane of FIG. 38, and is aligned with the vertical. In an exemplary embodiment, the conduit can move in the longitudinal direction of the conduit in one or more of those three planes. Note also that the aforementioned movements can also correspond to any the other conduits and/or the optical cables and/or drill bits for that matter in at least some exemplary embodiments. And in some embodiments these movements can be controlled to within at least 30, 25, 20, 15, 10 or 5 degrees or any value or range of values therebetween in 1° increments, and in some embodiments, these movements can be controlled to within 15, 14, 13, 12, 11, 10, 9, 8, 7, 6, 5, 4, 3, 2, 1, 0.75 or 0.5 mm or any value or range of values therebetween in 0.1 mm increments.

In an exemplary embodiment, the conduit 3834 can be spring loaded such that upon relief of a restraint, the conduit springs outward in a position shown. In an exemplary embodiment, this can be executed after the termination 3860 is positioned within the round window niche in a utilitarian position. Upon such positioning, a holding component can be moved to release the conduit so that it springs out of position shown. In an alternate embodiment, orientation and location of the conduit can be controlled such as utilizing guidewires and/or MEMS devices. Any device system and/or method that can enable movements of the conduits and or the other components detailed herein can utilize at least some exemplary embodiments.

In an exemplary embodiment, a material that dissolves or otherwise degrades when exposed to fluid, including air for that matter, is utilized to restrain the conduit and/or the other components. In an exemplary embodiment, just prior to insertion through the tympanic membrane, the component can be exposed, and then after a certain period of time exposed to the fluids inside the middle ear, the material degrades so that it releases the conduit and/or the optical cable. In an exemplary embodiment, a fluid can be purposely channeled to this material, which fluid dissolves material, thus releasing the conduit etc. In an exemplary embodiment, the material can be a material that dissolves upon the exposure to warm sterile water or saline based substances, etc.

FIG. 38 also shows light capture apparatus 3839. Here, the light capture apparatus is located inside the termination 3860, and the light capture features thereof are arranged so that it has a view of the tympanic membrane in the arrangement shown in FIG. 38. In this embodiment, the light capture apparatus 3839 is hard mounted in the termination 3860 (in the lumen). Movement of the termination will move the focus with the direction of light capture accordingly. In an exemplary embodiment, the light capture apparatus can be located at the distal end as shown, where light capture apparatus 3899 is located, facing left. Note that the light capture apparatuses could be the working end of fiber-optic cables, or electronic devices that convert the light that is captured into an electronic signal that is provided by wires to a display or the like or some other electrical based device. In one or more these embodiments, working in the light capture apparatus can be adjusted in the various degrees of freedom and/or with respect to the movements detailed above with respect to the conduit so as to “point” light capture apparatus in a desired direction. Note also that the embodiment of FIG. 38 presents the concept that multiple channels of light capture can be utilized. In an exemplary embodiment, there can be three or more images displayed simultaneously or otherwise available to the surgeon or other healthcare professional (the surgeon can toggle between the images). For example, an additional light capture apparatus could be located at the end of the of the termination 3860 the faces downward. Accordingly, the surgeon or other healthcare professional can have a view pointing in the longitudinal axis of the termination and a view or more than one view in the lateral direction, or multiple views in different lateral directions.

Note also that in an exemplary embodiment, the optical cable can be combined with the conduit. In an exemplary embodiment, the optical cable could also include a conduit collocated therewith. In this regard, in an exemplary embodiment, the very end of the optical cable could have a sharp conduit that can be utilized to pierce the round window, in the view that the surgeon sees would be the view of the conduit approaching the round window. The light capture portion of the cable could be offset/away from the end.

In view of the above, it can be seen that in some exemplary embodiments, there are a plurality of light capture devices.

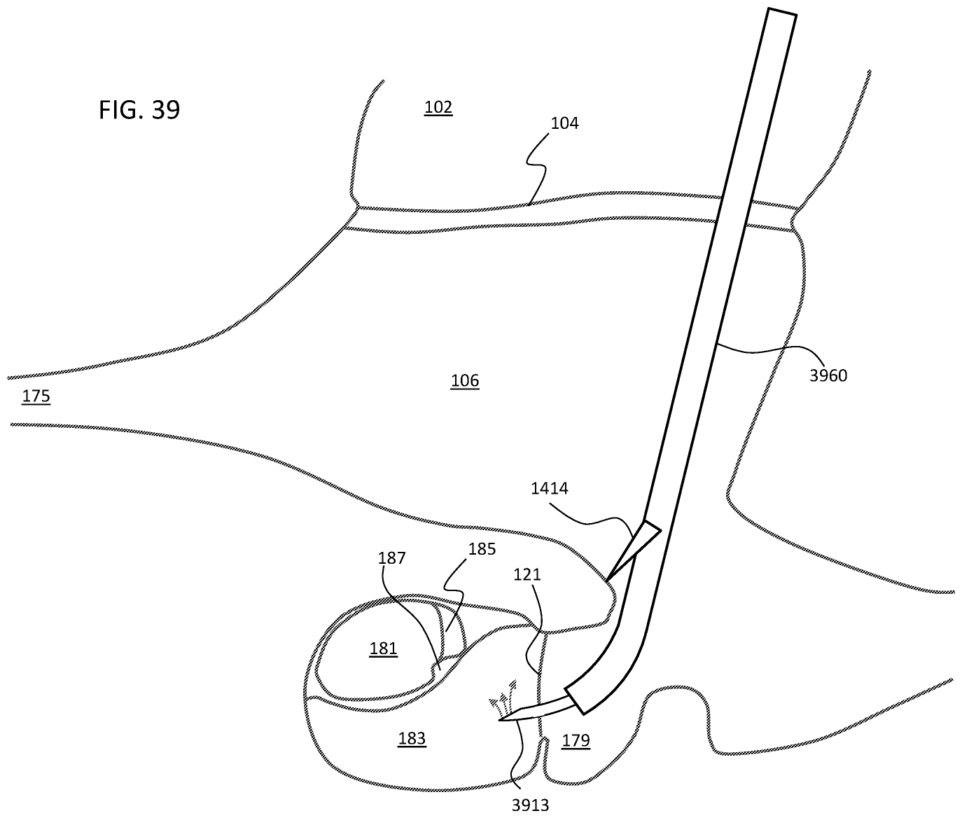

FIG. 39 presents an exemplary embodiment where the distal portions of the termination 3960 are curved and/or can be controllably curved or otherwise adjusted so that it has a trajectory that is angled away from the main longitudinal axis of the termination in general, and the longitudinal axis of the termination at the location where the termination extends through the tympanic membrane in some embodiments. Also as can be seen is a more relative size of the conduit 3913 that extends through the termination 3960. In this exemplary embodiment, the termination in general, and the flexure/bending of the termination in particular, is the part that controls the trajectory of the conduit 3913. In an exemplary embodiment, a combination of the two components can be utilized to control the trajectory of the conduit 3913.

In an exemplary embodiment, the endoscope is arranged or otherwise configured so that the distal tip of the termination and/or the distal tip of the conduit and/or the distal tip of the optical cable, etc., can be controllably positioned or otherwise purposely position so that the local longitudinal axis thereof is normal to the round window or otherwise at an angle from normal that is no more than 40, 35, 30, 25, 20, 19, 18, 17, 16, 15, 14, 13, 12, 11, 10, 9, 8, 7, 6, 5 4, 3, 2 or 1 degree from normal in a 50 percentile human factors engineering male and/or female that is a citizen of the United States of America and/or the European Union and/or the United Kingdom and/or Australia and/or New Zealand and/or Japan and/or the People's Republic of China on Feb. 21, 2021, of age 25 to age 45 and/or of age 45 to age 65 age 65 to age 85 or any value or range of values therebetween in one year increments, as of It is briefly noted that any one or more of the teachings detailed herein, unless otherwise noted, are also applicable to such types of humans. In an exemplary embodiment, the detailed herein can be applicable to an infant that is 1, 2, 3, 4, 5, 6, 7, 8, 9, 10, 11, 12, 13, 14, 15, 16, 17, 18 19 or 20 months old, or a child that is 1, 2, 3, 4, 5, 6, 7, 8, 9, 10, 11, 12, 13, 14, 15, 16 years old, etc., having the above noted human factors dates.

It is noted that any reference herein to a therapeutic substance corresponds to a disclosure of an active substance such as an active drug or an active biologic etc., and any disclosure herein to an active substance such as an active drug or the phrase active substance in the generic manner corresponds to a disclosure of an active biologic or a therapeutic substance, etc. Any active pharmaceutical ingredient that can have utilitarian value can be a therapeutic substance. Proteins can be therapeutic substances as well. It is also noted that in an at least some exemplary embodiments, an inactive fluid can be a physiological saline, which can be utilized to convey the therapeutic substance into the cochlea. In an exemplary embodiment, the therapeutic substance can be an artificial perilymph and/or a fluid comprising utilitarian ions and/or proteins for utilitarian cochlea health etc. In an exemplary embodiment, the therapeutic substance could be steroids, such as that utilized to treatment ears disease.

It is noted that therapeutic substances can be those detailed above with respect to the embodiment of FIG. 5.

In an exemplary embodiment, therapeutic substance include but are not limited to, any of those detailed above, and can include peptides, biologics, cells, drugs, neurotrophics, etc. Any substance that can have therapeutic features if introduced to the cochlea can be utilized in some embodiments.

Herein, therapeutic substance can be solutions, suspensions, gels and solid drug formulations. It is noted that unless otherwise stated or unless the art does not enable such, the delivery of the various therapeutic substance in the various states is executed and/or can be executed via puncturing or incising through the round window and/or through the promontory.

To be clear, delivery of solid therapeutic substances can be executed via the conduit embodiments/lumen embodiments detailed herein, which can be needle type device delivery. In some embodiments, a solid drug releasing PLGA implant is directed through a conduit or the like. In some embodiments, the solid therapeutic substances can be delivered through the cochleostomy, and in some embodiments, the solid therapeutic substances are delivered to the round window.

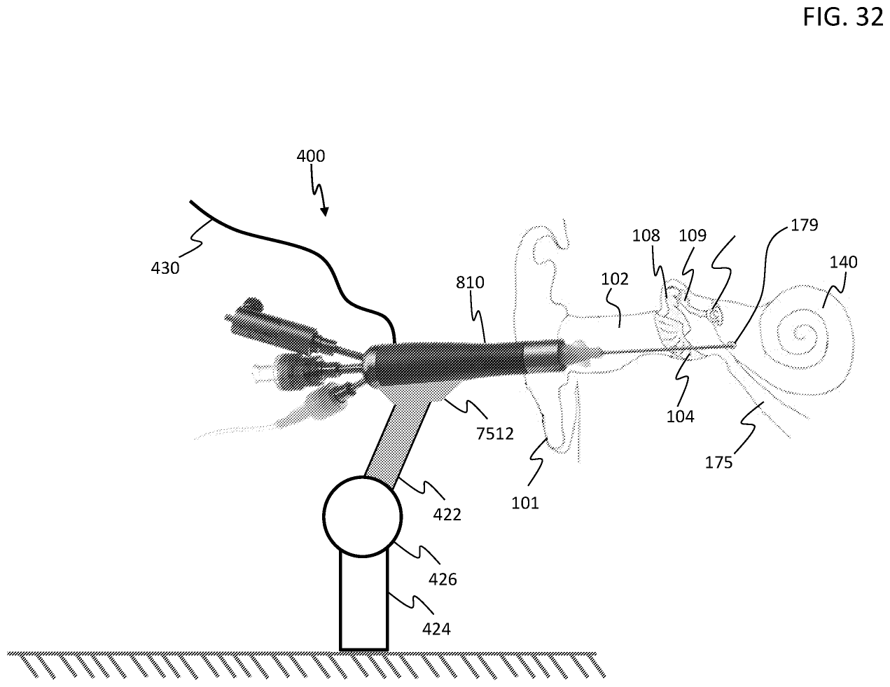

It is noted that at least some exemplary embodiments include utilization of the tools herein and/or variations thereof with a robotic system. In this regard, FIG. 32 is a perspective view of an exemplary embodiment of a therapeutic substance delivery system 400. It is noted that the embodiment depicted in FIG. 32 is presented for conceptual purposes only. Features are provided typically in the singular so as to demonstrate the concept associated therewith. However, it is noted that in some exemplary embodiments, some of these features are duplicated, triplicated, quadplicated, etc. so as to enable the teachings detailed herein and/or variations thereof. Briefly, it is noted that any teaching detailed herein can be combined with a robotic apparatus and/or a robotic system according to the teachings detailed herein and/or variations thereof. In this regard, any method action detailed herein corresponds to a disclosure of a method action executed by a robotic apparatus and/or utilizing a robot to execute that action and/or executing that method action is part of a method where other actions are executed by robot and/or a robotic system etc. Still further, it is noted that any apparatus detailed herein can be utilized in conjunction with a robotic apparatus and/or a robot and/or a system utilizing such. Accordingly, any disclosure herein of an apparatus corresponds to a disclosure of an apparatus that is part of a robotic apparatus and/or a robotic system etc. and/or a system that includes a robotic apparatus etc.

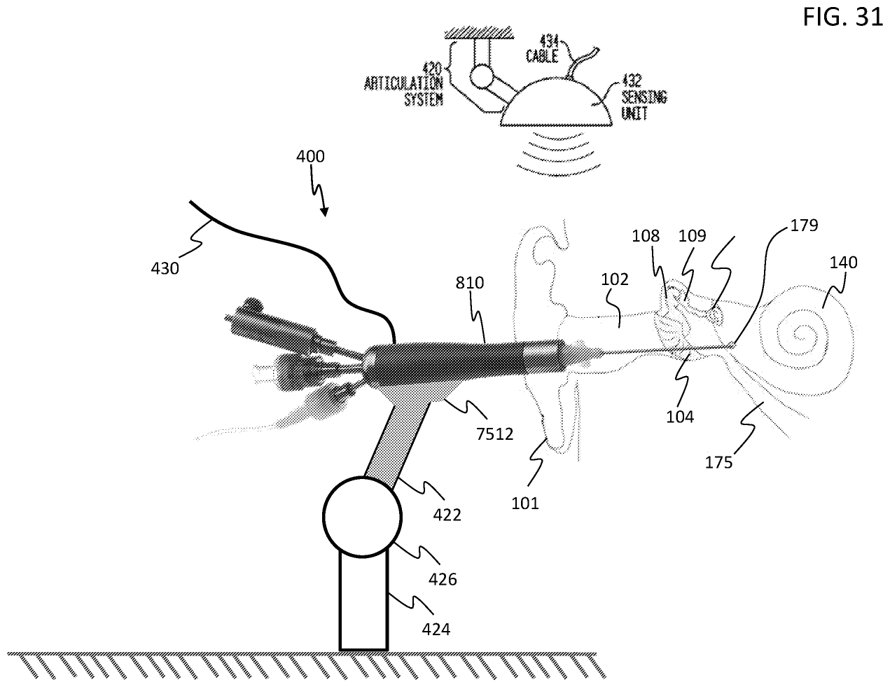

System 400 includes a robotic insertion apparatus having the ear endoscope 810 including a releasable connection to mount 7512, which is supported by a support and movement system, comprising support arm 422 which is connected to joint 426 which in turn is connected to support arm 424. Support arm 424 is rigidly mounted to a wall, a floor, or some other relatively stationary surface. That said, in an alternative embodiment, support arm 424 is mounted to a frame that is attached to the head of the recipient or otherwise connected to the head of the recipient such that global movement of the head will result in no relative movement of the system 400 in general, and the endoscope in particular, relative to the tissue of the human, such as the cochlea, or tympanic membrane, or round window, or promontory, etc. Joint 426 permits arm 2510, and thus the endoscope, to be moved in one, two, three, four, five, or six degrees of freedom. (It is noted again that FIG. 31 is but a conceptual FIG.—there can be joints located along the length of various components, such as for example arm 4222 enable that to articulate in the one or more of the aforementioned degrees of freedom at those locations. In an exemplary embodiment, joint 426 includes actuators that move mount 7512, and thus the endoscope, in an automated manner, as will be described below. In an exemplary embodiment, the system is configured to be remotely controlled via communication with a remote control unit via communication lines of cable 430. In an exemplary embodiment, the system is configured to be automatically controlled via a control unit that is part of the system 400. Additional details of this will be described below.

The system 400 further includes by way of example only and not by way of limitation, sensor/sensing unit 432. That said, in some embodiments, sensor 432 is not part of system 400. In some embodiments, it is a separate system. Still further, in some embodiments, it is not utilized at all with system 400. While sensor 432 is depicted as being co-located simultaneously with the endoscope, etc., as detailed below, sensor 432 may be used relatively much prior to use of the endoscope. Sensing unit 432 is configured to scan the head of a recipient and obtain data indicative of spatial locations of internal organs (e.g., mastoid bone 221, middle ear cavity 423 and/or ossicles 106, etc.) In an exemplary embodiment, sensing unit 432 is a unit that is also configured to obtain data indicative of spatial locations of at least some components of the endoscope and/or other components of the robotic apparatus attached thereto. The obtained data may be communicated to remote control unit 440 via communication lines of cable 434. As may be seen, sensor 432 is mounted to a support and movement system 420 that may be similar to or the same as that used by the robotic apparatus supporting the endoscope.

In an exemplary embodiment, sensing unit 432 is an MRI system, an X-Ray system, an ultrasound system, a CAT scan system, or any other system which will permit the data indicative of the spatial locations to be determined as detailed herein and/or variations thereof. As will be described below, this data may be obtained prior to surgery and/or during surgery. It is noted that in some embodiments, at least some portions of the endoscope are configured to be better imaged or otherwise detected by sensing unit 432. In an exemplary embodiment, the tip of the endoscope includes radio-opaque contrast material. The stop of the endoscope can also include such radio-opaque contrast material. In an exemplary embodiment, at least some portions of endoscope in general, and the robotic system in particular, or at least the arm 7510, mount 7512, arm 422, etc., are made of non-ferromagnetic material or other materials that are more compatible with an MRI system or another sensing unit utilized with the embodiment of FIG. 66 than ferromagnetic material or the like. As will be described in greater detail below, the data obtained by sensing unit 432 is used to construct a 3D or 4D model of the recipient's head and/or specific organs of the recipient's head (e.g., temporal bone) and/or portions of the robotic apparatus of which the endoscope is a part. That said, to be clear, in some embodiments, sensing unit 432 is not present, as seen in FIG. 32.

It is also noted that in some exemplary embodiments of system 400, there are actuators or the like that drive the endoscope into the ear structure. These actuators can be in signal communication with the control unit. In an exemplary embodiment, the control unit can control the actuators to push the into and/or out of the ear system as will be described in greater detail below. Concomitant with the robotic assembly supporting the endoscope, in an exemplary embodiment, the control unit is configured to automatically control these actuators.

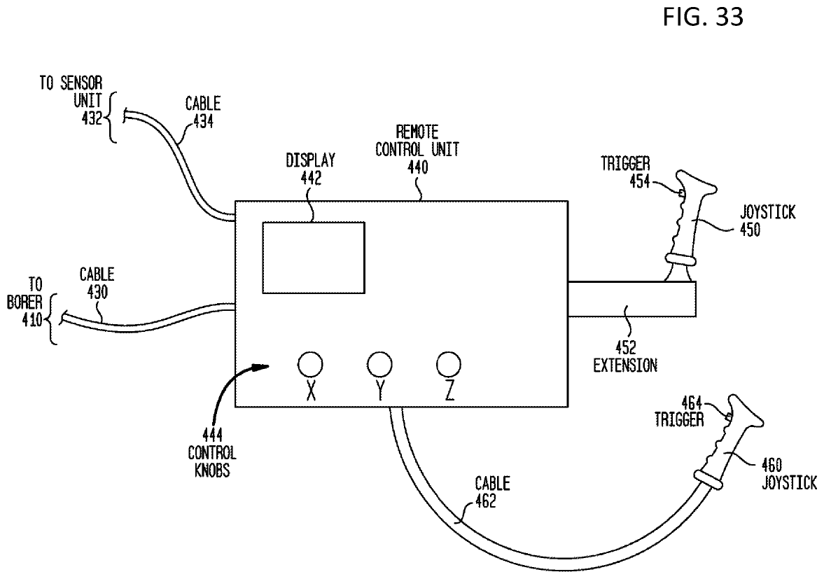

FIG. 33 is a simplified block diagram of an exemplary embodiment of a remote control unit 440 for controlling the robotic apparatus supporting the endoscope and sensing unit 432 via communication lines 430 and 434, respectively. Again, it is noted that in some alternate embodiments, the remote control unit 440 is an entirely automated unit. That said, in some alternate embodiments, the remote control unit can be operated automatically as well as manually, which details will be described below.

Remote control unit 440 includes a display 442 that displays a virtual image of the tissue obtained from sensor 432 and/or component(s) of the endoscope and may superimpose a virtual image of the insertion apparatus onto the virtual image indicative of a current position of the tool relative to the ear anatomy. An operator (e.g., surgeon, certified healthcare provider, etc.) utilizes remote control unit 440 to control some or all aspects of the robotic apparatus and/or sensing unit 432. Exemplary control may include depth of insertion, angle of insertion, speed of advancement and/or retraction of the tool (endoscope for example), etc. (It is noted that any reference to advancement and/or retraction also corresponds to an alternate disclosure of lateral movement and/or rotation of the tool and/or a change in the angle on any one or more of the three planes relative to the tissue that is the target, etc.) Such control may be exercised via joystick 450 mounted on extension 452 which fixedly mounts joystick 450 to a control unit housing. Such control may be further exercised via joystick 460 which is not rigidly connected to housing of remote control unit 440. Instead, it is freely movable relative thereto and is in communication with the remote control unit via communication lines of cable 462. Joystick 462 may be part of a virtual system in which the remote control unit 440 extrapolates control commands based on how the joystick 462 is moved in space, or joystick may be a device that permits the operator more limited control over the cavity borer 410. Such control may include, for example an emergency stop upon release of trigger 464 and/or directing the robot to drive the endoscope further into the ear system, etc. by squeezing the trigger 464 (which, in some embodiments, may control a speed at which the endoscope is advanced by squeezing harder and/or more on the trigger). In the same vein, trigger 454 of joystick 450 may have similar and/or the same functionality.

Control of the robot assembly supporting the endoscope may also be exercised via knobs 440 which may be used to adjust an angle of the endoscope in the X, Y and Z axis, respectively. Other controls components may be included in remote control 440.

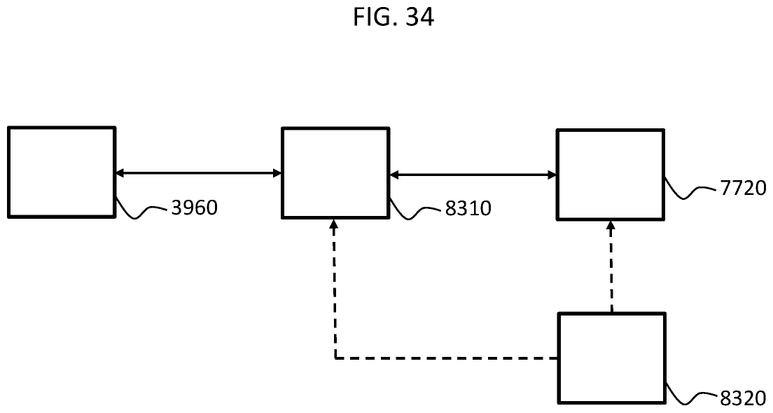

FIG. 34 depicts an exemplary functional schematic of an exemplary system that includes a data collection unit 3960 that receives data from, for example, the sensors of the endoscope, in signal communication with a control unit 8310 which is in turn in signal communication with an actuator assembly, 7720, where the actuator assembly 7720 is a proxy for a component that positions the endoscope, or at least advances and/or retracts the endoscope. The data collection unit and the control unit can be one and the same in some embodiments.

It is also noted that in some embodiments, the there is no control unit. That is, the system can be a purely data collection system, which conveys information to the surgeon or other healthcare professional to instruct (e.g., the output of the control unit and/or the test unit can be instead an instruction as opposed to a control signal) or otherwise provide an indication of the phenomenon to the surgeon or other healthcare professional.

Also functionally depicted in FIG. 34 is the optional embodiment where an input device 8320 is included in the system (e.g., which could be on an embodiment where the actuator assembly 7720 is connected to the endoscope, but the input device 8320 is located remote from the endoscope, which could be part of a remote unit 440). In an exemplary embodiment, the input device 8320 could be the trigger 454 and/or 464 of the remote control unit 440. Again, in an exemplary embodiment, the input device 8320 can be utilized to enable advancement and/or withdrawal of the endoscope and the system 400 could control the advancement and/or withdrawal based on an automated protocol or some other flyby wire type system. In the embodiment of FIG. 34, the input device 8320 can be in signal communication directly to the endoscope, and/or in signal communication with the control unit 8310.

In an exemplary embodiment, control unit 8310 can correspond to the remote unit 440. That said, in an alternate embodiment, remote unit 440 can be a device that is in signal communication with control unit 8310. Indeed, in an exemplary embodiment, input device 8320 can correspond to remote control unit 440.

More particularly, control unit 8310 can be a signal processor or the like or a personal computer or the like or a mainframe computer or the like etc., that is configured to receive signals from the data collection unit 3960 and analyze those signals to evaluate an insertion status of the endoscope. More particularly, the control unit 8310 can be configured with software the like to analyze the signals from unit 3960 in real time and/or in near real time as the endoscope is being advanced by an actuator assembly of the robotic system. The control unit 8310 analyzes the input from test unit 3960 as the endoscope is advanced by the actuator assembly for example, and evaluates the input to determine if there exists an undesirable insertion status and/or evaluates the input to determine if the input indicates that a scenario could occur or otherwise there exists data in the input that indicates that a scenario is more likely to occur relative to other instances where the insertion status of the endoscope will become undesirable if the endoscope is continued to be advanced into the ear system, all other things remaining the same (e.g., insertion angle/trajectory, etc., which can be automatically changed as well via—more on this below). In an exemplary embodiment, upon such a determination, control unit 8310 could halt the advancement of the endoscope by stopping the actuator(s) of actuator assembly and/or could slow the actuator(s) so as to slow rate of advancement of the endoscope and/or could reverse the actuator(s) so as to reverse or otherwise retract the endoscope (either partially or fully). In at least some exemplary embodiments, control unit 8310 can be configured to override the input from input unit 8320 input by the surgeon or the user or the like of the systems herein.

In an exemplary embodiment, the outputs of unit 3960 corresponds to the outputs indicated herein. Alternatively, and/or in addition to this, input into control unit 8310 can flow from other sources. Any input relating to the measurement of voltage associated executing the teachings herein into control unit 8310 can be utilized in at least some exemplary embodiments.

In an exemplary embodiment, control unit 8310 can be configured to determine, based on the input from test unit 3960, whether the endoscope has come into contact with the tympanic membrane and/or the round window and/or the promontory, etc., and/or that one or more anomalous endoscope positions has occurred and/or whether there exists an increased likelihood that such will occur, and automatically control the actuator assembly of the insertion system accordingly. In an exemplary embodiment, control unit 8310 does not necessarily determine that such an insertion status exists or is more likely to exist, but instead is programmed or otherwise configured so as to control the actuator assembly 7720 according to a predetermined regime based on the input from the test unit 3960. That is, the control unit 8310 need not necessarily “understand” otherwise “know” the actual insertion status or the forecasted insertion status of the endoscope, but instead need only be able to control the actuator assembly 7720 based on the input.

In an exemplary embodiment, control unit 8310 can be configured to determine, based on the input from test unit 3960, the insertion depth of the endoscope and/or a forecasted insertion depth of the endoscope, and automatically control the actuator assembly 7720 accordingly. In an exemplary embodiment, control unit 8310 does not necessarily determine the insertion depth or forecasted insertion depth, but instead is programmed or otherwise configured so as to control the actuator assembly 7720 according to a predetermined regime based on the input from the test unit 3960. That is, the control unit 8310 need not necessarily “understand” otherwise “know” the actual insertion depth or the forecasted insertion depth of the endoscope, but instead need only be able to control the actuator assembly 7720 based on the input.

In an exemplary embodiment, control unit 8310 can be configured to determine, based on the input from test unit 3960, executing, for example, the methods/techniques disclosed herein, whether the endoscope has buckled or has become hung up or has adopted an unutilitarian trajectory and/or position and/or any other anomalous endoscope location as disclosed herein or otherwise may be the case and/or whether there exists an increased likelihood that such will occur, and automatically control the actuator assembly 7720 accordingly. In an exemplary embodiment, control unit 8310 does not necessarily determine that such, exists or is more likely to exist, but instead is programmed or otherwise configured so as to control the actuator assembly 7720 according to a predetermined regime based on the input from the test unit 3960. That is, the control unit 8310 need not necessarily “understand” otherwise “know” that the condition has occurred or will occur in the future, but instead need only be able to control the actuator assembly 7720 based on the input.

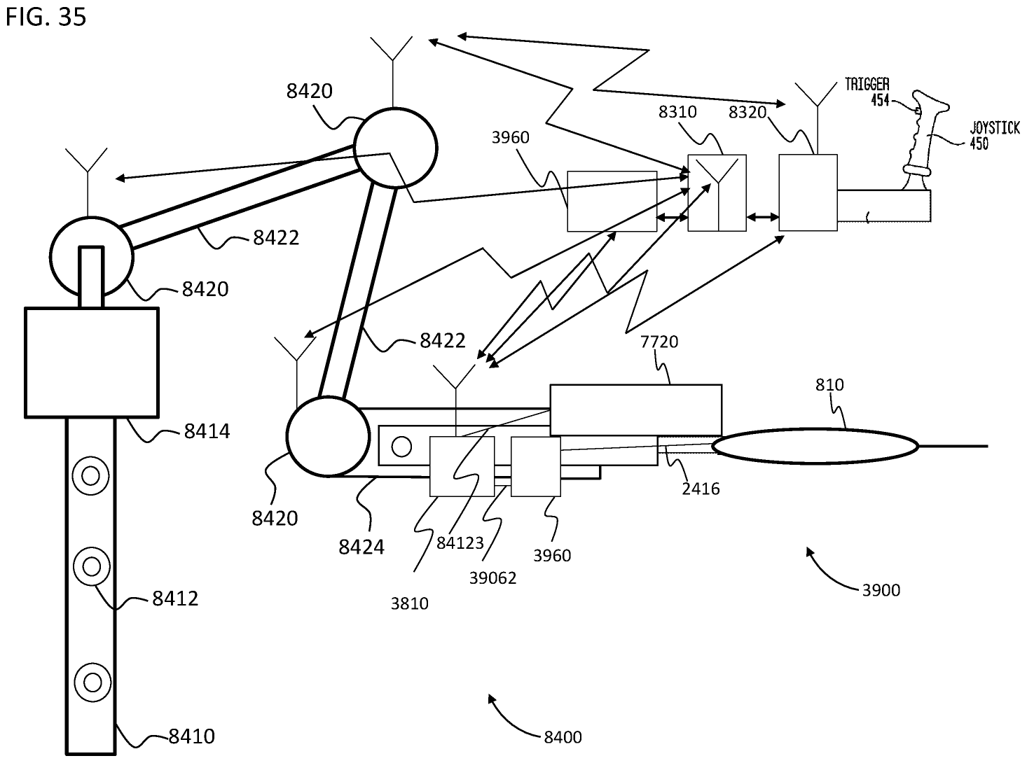

To be clear, while the embodiment detailed above is focused on controlling the actuator assembly 7720 based on data from the system so as to control the advancement and/or retraction of the endoscope based on the data disclosed herein and, in an alternate embodiment, the system 400 controls one or more other actuators of the robot apparatus of system 400. These one or more other actuators can be exclusive from the actuator assembly 7720, or can include the actuator assembly 7720. In this regard, FIG. 35 depicts an exemplary robot apparatus 8400, that includes the endoscope detailed above and/or variations thereof with respect to the integration of a system disclosed herein therewith mounted on arm 8424 utilizing bolts in a manner concomitant with that detailed above. In an exemplary embodiment, robot apparatus 8400 has the functionality or otherwise corresponds to that of the embodiment of FIG. 33. In this regard, any functionality associated or otherwise described with respect to the embodiment of FIG. 33 corresponds to that of the embodiment of FIG. 35, and vice versa. In this exemplary embodiment, the actuator apparatus 7720 is in signal communication with unit 3810 via electrical lead 84123. In this regard, signals to and/or from the actuator assembly 7720 can be transmitted to/from the antenna of unit 8310 (the “Y” shaped elements are antennas) and thus communicated via lead 84123. It is briefly noted that while the embodiment depicted in FIG. 35 utilizes radiofrequency communication, in alternate embodiments, the communications can be wired. In an exemplary embodiment both can be utilized.

The robot apparatus 8400 includes a recipient interface 8410 which entails an arch or halo like structure made out of metal or the like that extends about the recipient's cranium or other parts of the body. The interface 8410 is bolted to the recipient's head via bolts 8412. That said, in alternate embodiments, other regimes of attachment can be utilized, such as by way of example only and not by way of limitation, strapping the robot to the recipient's head. In this regard, the body and interface 8410 can be a flexible strapping can be tightened about the recipient's head.

Housing 8414 is located on top of the interface 8410, as can be seen. In an exemplary embodiment, housing 8414 includes a battery or the like or otherwise provides an interface to a commercial/utility power supply so as to power the robot apparatus. Still further, in an exemplary embodiment, housing 8414 can include hydraulic components/connectors to the extent that the actuators herein utilize hydraulics as opposed to and/or in addition to electrical motors. Mounted on housing 8414 is the first actuator 8420, to which arm 8422 is connected in an exemplary embodiment, actuator 8420 enables the components “downstream” (i.e., the arm connected to the actuator, and the other components to the endoscope) to articulate in one, two, three, four, five or six degrees of freedom. A second actuator 8420 is attached to the opposite end of the arm 8422, to which is attached a second arm 8422, to which is attached a third actuator 8420, to which is attached to the endoscope attachment structure 8424. Elements 8422 and 8424 can be metal beams, such as I beams or C beams or box beams, etc. actuators 8420 can be electrical actuators and/or hydraulic actuators.

As can be seen, each actuator 8420 is provided with an antenna, which antenna is in signal communication with the control unit 8310. In an exemplary embodiment, control unit 8310 can control the actuation of those actuators 8420 so as to position the endoscope 3900 (generically identified—reference numeral 810 is also used) at the desired position relative to the recipient. That said, in an alternate embodiment, a single antenna can be utilized, such as one mounted on housing 8414, which in turn is connected to a decoding device that outputs a control signal, such as a driver signal based on the decoded RF signal, to the actuators 8420 (as opposed to each actuator having such a device), which control signals can be provided via a wired system/electrical leads extending from housing 8414 to the actuators. Note also that in some alternate embodiments, control unit 8310 is in wired communication with the actuators, either directly or indirectly, and/or is in wired communication with the decoding device located in the housing 8414. Any arrangement that can enable control of the robot apparatus in general, and the actuators thereof in particular, via control unit 8310 can be utilized in at least some exemplary embodiments.

Note also that while the embodiment depicted in FIG. 35 is such that the actuators 8420 must actuate so as to extend the endoscope into the body, in an alternate embodiment, as noted above, the endoscope can be mounted on a rail system or the like, wherein a cylindrical actuator or the like pushes the endoscope in a linear manner into the head and withdrawals the endoscope in the linear manner from the head. In an exemplary embodiment, this actuator apparatus can enable one degree of freedom movements of the endoscope, while in other embodiments, this actuator apparatus can enable two or three or four or five or six degrees of freedom. Indeed, in an exemplary embodiment, this actuator apparatus can enable movement only in a linear direction, but can enable rotation of the endoscope about the longitudinal axis thereof. Any arrangement of actuator assemblies that will enable the endoscope to be positioned relative to the ear system via robotic positioning thereof can be utilized in at least some exemplary embodiments.

Any control unit and/or test unit or the like disclosed herein can be a personal computer programs was to execute one or more or all of the functionalities associated there with are the other functionalities disclosed herein. In an exemplary embodiment, any control unit and/or test unit or the like can be a dedicated circuit assembly configured so as to execute one or more or all of the functionalities associated there with or the other functionalities disclosed therein. In an exemplary embodiment, and the control unit and/or test unit or the like disclosed herein can be a processor or the like or otherwise can be a programmed processor.

FIG. 36 depicts another exemplary embodiment, as seen. FIG. 36 presents such an exemplary embodiment, with the links between the antennas removed for clarity. Testing system 4044 detailed shown in signal communication with control unit 8310. In this exemplary embodiment, system 4044 corresponds to that detailed above vis-à-vis determining anomalous endoscope location with the exception that it is entirely divorced from the endoscope, save for the communication between system 4044 and the control unit 8310, to the extent such is relevant for the purposes of discussion, where control unit 8310 is in signal communication with one or more of the assemblies of the robot apparatus, such as the actuator assembly 7720. Here, during insertion, and/or prior to insertion and/or after insertion, the system 4044 monitors or otherwise measures phenomenon detailed herein and communicates those measurements and/or the analysis thereof to control unit 8310, which analyzes those signals and develops a control regime for endoscope insertion and/or endoscope positioning based on those signals. Note also that in some exemplary embodiments, the system 4044 can have multiple measurement sensors, some of which are part of the robot apparatus, and some of which are separate from the robot apparatus, all of which are part of system 4044. Alternatively, these various components of the system 4044 can communicate with test unit 3960. Such can have utilitarian value with respect to a scenario where measurements are first taken prior to placing the endoscope near the pertinent tissues and after inserting the endoscope into the ear system, where it is undesirable to have the endoscope proximate certain tissue. Any device, system, and/or method that will enable controlled movement of the endoscope relative to the ear system and/or cochlea based on phenomenon associated with the recipient can be utilized in at least some exemplary embodiments.

Again, the test unit and the system 4044 can be one and the same in some embodiments, and in some embodiments, functionality can be bifurcated between the two as separate units. Indeed, element 4044 in FIG. 36 can be a proxy for the control unit and/or the test units detailed above.

It is briefly noted that any reference to an endoscope and/or a drill bit and/or a hand tool herein corresponds to a disclosure of an alternate embodiment that includes that feature in a more generic tool and/or in another of the tools disclosed herein, and visa-versa.

It is noted that any disclosure of a device and/or system herein corresponds to a disclosure of a method of utilizing such device and/or system. It is further noted that any disclosure of a device and/or system herein corresponds to a disclosure of a method of manufacturing such device and/or system. It is further noted that any disclosure of a method action detailed herein corresponds to a disclosure of a device and/or system for executing that method action/a device and/or system having such functionality corresponding to the method action. It is also noted that any disclosure of a functionality of a device herein corresponds to a method including a method action corresponding to such functionality. Also, any disclosure of any manufacturing methods detailed herein corresponds to a disclosure of a device and/or system resulting from such manufacturing methods and/or a disclosure of a method of utilizing the resulting device and/or system.

Unless otherwise specified or otherwise not enabled by the art, any one or more teachings detailed herein with respect to one embodiment can be combined with one or more teachings of any other teaching detailed herein with respect to other embodiments, and this includes the duplication or repetition of any given teaching of one component with any like component. Also, embodiments include devices systems and/or methods that explicitly exclude any one or more of a given teaching herein. That is, at least some embodiments include devices systems and/or methods that explicitly do not have one or more of the things that are disclosed herein.

While various embodiments of the present invention have been described above, it should be understood that they have been presented by way of example only, and not limitation. It will be apparent to persons skilled in the relevant art that various changes in form and detail can be made therein without departing from the scope of the invention.

1. A device, comprising:
an ear system endoscope configured to incise through tissue to reach duct(s) of an inner ear of a human and to deliver a therapeutic substance to the duct(s) through a resulting incision.

2. The device of claim 1, wherein:
the device includes a conduit configured to incise through tissue to reach the duct(s), the conduit having a lumen therein through which the therapeutic substance can be delivered to the duct(s).

3. The device of claim 1, wherein:
the device includes a drill bit configured to incise through tissue to reach the duct(s), the drill bit having a lumen therein through which the therapeutic substance can be delivered to the duct(s).

4. The device of claim 1, wherein:
the device includes a component configured to fix a distal end of the device in or proximate to a round window niche of the human, which component is separate from a sharp end of a termination of the device.

5. The device of claim 1, wherein:
the device includes a conduit with a sharp end configured to pierce a round window of the human to reach the duct(s) of the inner ear.

6. The device of claim 1, wherein:
the device is a handheld device configured to reach the duct(s) via a route that extends through a tympanic membrane and then across a middle ear to at least one of a promontory within a middle ear of the human or a round window niche of the human.

7. (canceled)

8. The device of claim 1, wherein:
the device includes a component configured to fix a distal end of the device in or proximate to a round window niche of the human; and
the device includes a component configured to rotate relative to the fixed distal.

9-11. (canceled)

12. A system, comprising:
a processor;
a sensor; and
an output device, wherein
the system is configured to automatically process data from the sensor indicative of sensed tissue of an ear system of a human sensed by the sensor and, using the processor, automatically identify a location in the ear system for an incision to access an area of the ear system behind the location, and provide output indicative of the identified location.

13. The system of claim 12, wherein:
the sensor is an optical sensor.

14. The system of claim 12, wherein:
the output is a visible indication projected on the location.

15. The system of claim 12, wherein:
the sensor is an optical sensor;
the output device is a display;
the system is configured to display an image of the location obtained with the optical sensor on the display, and overlay the identified location on the image.

16. The system of claim 15, wherein:
the display is located within 100 inches from the sensor.

17. The system of claim 12, wherein:
the tissue of the ear system is at least part of an ossicles of a human; and
the system is configured to sense the location of the at least part of the ossicles through an intact tympanic membrane.

18-30. (canceled)

31. A device, comprising:
a therapeutics substance delivery apparatus; and
an incisor, wherein
the device is a minimally invasive inner ear therapeutic substance delivery device configured to reach the inner ear through a passage through the tympanic membrane no greater than 10 mm 5 mm in diameter to incise through tissue and deliver a therapeutic substance, and
the incisor is at least one of a drill bit configured to drill through a promontory of a cochlea of a human, or a conduit configured to pierce a round window of the human with a fully intact round window niche.

32. The device of claim 31, wherein:
the therapeutic substance delivery apparatus is configured to deliver a solid therapeutic substance to the cochlea.

33. The device of claim 31, wherein:
the therapeutic substance delivery apparatus is configured to deliver a fluid therapeutic substance to the cochlea.

34. The device of claim 31, wherein:
the incisor is the drill bit.

35. The device of claim 31, wherein:
the incisor is a chamfered portion of the conduit; and
the device is configured to deliver the therapeutic substance through the conduit.

36. The device of claim 31, further comprising:
an optical scope configured to at least view a distal end of the device.

37. The device of claim 31, wherein:
the incisor is the drill bit, and the drill bit is a spherical drill bit.
